(19) United States
(12) Patent Application Publication
(10) Pub. No.: US 2002/0147334 A1
(43) Pub. Date: Oct. 10, 2002

(54) Vitronectin Receptor Antagonists

(75) Inventors: William H. Miller, Collegeville, PA (US); John G. Gleason, Downingtown, PA (US); Dirk Heerding, Malvern, PA (US); James M. Samanen, Phoenixville, PA (US); Irene N. Uzinskas, Villanova, PA (US); Peter J. Manley, Phoenixville, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/023,471

(22) Filed: Feb. 26, 2002

Related U.S. Application Data
(63) Continuation of application No. 09/623,827, filed on Sep. 8, 2000, now abandoned.
Application No. 09/623,827 is a 371 of international application No. PCT/US99/05232, filed on Mar. 10, 1999.
(60) Provisional application No. 60/077,610, filed on Mar. 10, 1998.
(60) Provisional application No. 60/096,063, filed on Aug. 11, 1998.

(51) Int. Cl. C07D 471/02; C07D 277/28; C07D 263/48; C07D 253/065; C07D 243/04; C07D 239/12

(52) U.S. Cl. 540/553; 544/182; 544/208; 544/224; 544/330; 544/336; 546/122; 548/190; 548/222

(57) ABSTRACT

Compounds of the formula (1) are disclosed which are vitronectin receptor antagonists and are useful in the treatment of osteoporosis:

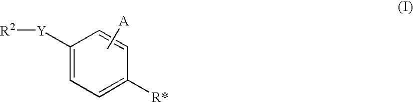

### FIELD OF THE INVENTION

This invention relates to pharmaceutically active compounds which inhibit the vitronectin receptor and are useful for the treatment of inflammation, cancer and cardiovascular disorders, such as atherosclerosis and restenosis, and diseases wherein bone resorption is a factor, such as osteoporosis.

### BACKGROUND OF THE INVENTION

Integrins are a superfamily of cell adhesion receptors, which are transmembrane glycoproteins expressed on a variety of cells. These cell surface adhesion receptors include gpIIb/IIIa (the fibrinogen receptor) and αᵥβ₃ (the vitronectin receptor). The fibrinogen receptor gpIIb/IIIa is expressed on the platelet surface, and mediates platelet aggregation and the formation of a hemostatic clot at the site of a bleeding wound. Philips, et al., Blood., 1988, 71, 831. The vitronectin receptor αᵥβ₃ is expressed on a number of cells, including endothelial, smooth muscle, osteoclast, and tumor cells, and, thus, it has a variety of functions. The αᵥβ₃ receptor expressed on the membrane of osteoclast cells mediates the adhesion of osteoclasts to the bone matrix, a key step in the bone resorption process. Ross, et al., J. Biol. Chem., 1987, 262, 7703. A disease characterized by excessive bone resorption is osteoporosis. The αᵥβ₃ receptor expressed on human aortic smooth muscle cells mediates their migration into neointima, a process which can lead to restenosis after percutaneous coronary angioplasty. Brown, et al., Cardiovascular Res., 1994, 28, 1815. Additionally, Brooks, et al., Cell, 1994, 79, 1157 has shown that an αᵥβ₃ antagonist is able to promote tumor regression by inducing apoptosis of angiogenic blood vessels. Thus, agents that block the vitronectin receptor would be useful in treating diseases, such as osteoporosis, restenosis and cancer.

The vitronectin receptor is now known to refer to three different integrins, designated αᵥβ₁, αᵥβ₃ and αᵥβ₅. Horton, et al., Int. J. Exp. Pathol., 1990, 71, 741. αᵥβ₁ binds fibronectin and vitronectin. αᵥβ₃ binds a large variety of ligands, including fibrin, fibrinogen, laminin, thrombospondin, vitronectin, von Willebrand's factor, osteopontin and bone sialoprotein I. αᵥβ₅ binds vitronectin. The vitronectin receptor αᵥβ₅ has been shown to be involved in cell adhesion of a variety of cell types, including microvascular endothelial cells, (Davis, et al., J. Cell. Biol., 1993, 51, 206), and its role in angiogenesis has been confirmed. Brooks, et al., Science, 1994, 264, 569. This integrin is expressed on blood vessels in human wound granulation tissue, but not in normal skin.

The vitronectin receptor is known to bind to bone matrix proteins which contain the tri-peptide Arg-Gly-Asp (or RGD) motif. Thus, Horton, et al., Exp. Cell Res. 1991, 195, 368, disclose that RGD-containing peptides and an anti-vitronectin receptor antibody (23C6) inhibit dentine resorption and cell spreading by osteoclasts. In addition, Sato, et al., J. Cell Biol. 1990, 111, 1713 discloses that echistatin, a snake venom peptide which contains the RGD sequence, is a potent inhibitor of bone resorption in tissue culture, and inhibits attachment of osteoclasts to bone.

It has now been discovered that certain compounds are potent inhibitors of the αᵥβ₃ and αᵥβ₅ receptors. In particular, it has been discovered that such compounds are more potent inhibitors of the vitronectin receptor than the fibrinogen receptor.

### SUMMARY OF THE INVENTION

This invention comprises compounds of the formula (I) as described hereinafter, which have pharmacological activity for the inhibition of the vitronection receptor and are useful in the treatment of inflammation, cancer and cardiovascular disorders, such as atherosclerosis and restenosis, and diseases wherein bone resorption is a factor, such as osteoporosis.

This invention is also a pharmaceutical composition comprising a compound according to formula (I) and a pharmaceutically carrier.

This invention is also a method of treating diseases which are mediated by the vitronectin receptor. In a particular aspect, the compounds of this invention are useful for treating atherosclerosis, restenosis, inflammation, cancer and diseases wherein bone resorption is a factor, such as osteoporosis.

### DETAILED DESCRIPTION

This invention comprises novel compounds which are more potent inhibitors of the vitronectin receptor than the fibrinogen receptor. This invention comprises compounds of formula (I):

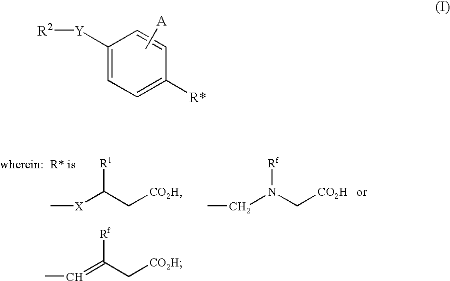

X is CR′R′, NR′, O or S;

Y is CR′R′, NR′, O or S;

A is H, halo, —OR^{g}, —SR^{g}, —CN, —NR^{g}R^{k}, —NO₂, —CF₃, —S(O)ᵣCF₃, —CO₂R^{g}, —COR^{g}, —CONR^{g}₂—C₁₋₆alkyl, —C₀₋₆alkyl-Ar, —C₀₋₆alkyl-Het, —C₀₋₆alkyl-C₃₋₆cycloalkyl, —S(O)ₖR^{g}, or CH₂N(R^{f})₂;

R¹ is —C₀₋₆alkyl-Het-, —C₀₋₆alkyl-Ar, —C₁₋₆alkyl, —H, —CN, —CH═CH₂, —C≡CH or, —S(O)ₖR^{g};

R²is

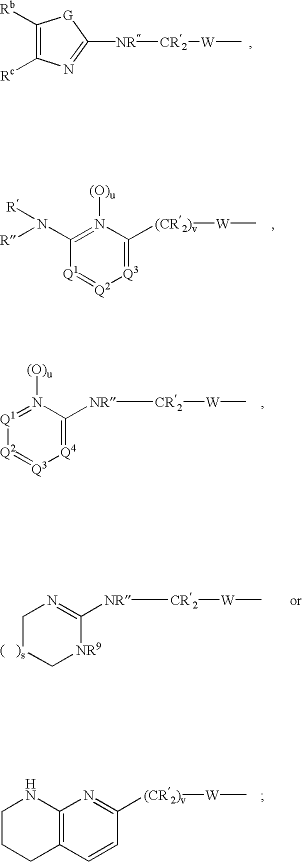

W is —(CHR^{g})ₐ—U—(CHR^{g})_{b}—;

U is absent or CO, CR^{g}₂, C(═CR^{g}₂), S(O)ₖ, O, NR^{g}, CR^{g}OR^{g}, CR^{g}(OR^{k})CR^{g}₂, CR^{g}₂CR^{g}(OR^{k}), C(O)CR^{g}₂, CR^{g}₂C(O), CONRⁱ, NRⁱCO, OC(O), C(O)O, C(S)O, OC(S), C(S)NR^{g}, NR^{g}C(S), S(O)₂NR^{g}, NR^{g}S(O)₂ N═N, NR^{g}NR^{g}, NR^{g}CR^{g}₂, CR^{g}₂NR^{g}, CR^{g}₂O, OCR^{g}₂, C≡C , CR^{g}═CR^{g}, Ar or Het;

G is NR^{e}, S or O;

R^{g} is H, C₁₋₆alkyl, Het-C₀₋₆alkyl, C₃₋₇cycloalkyl-C₀₋₆alkyl or Ar—C₀₋₆alkyl,

R^{k} is R^{g}, —C(O)R^{g}, or —C(O)OR^{f};

Rⁱ is is H, C₁₋₆alkyl, Het-C₀₋₆alkyl, C₃₋₇cycloalkyl-C₀₋₆alkyl, Ar—C₀₋₆alkyl, or C₁₋₆alkyl substituted by one to three groups chosen from halogen, CN, NR^{g}₂, OR^{g}, SR^{g}, CO₂R^{g}, and CON(R^{g})₂;

R^{f} is H, C₁₋₆alkyl or Ar—C₀₋₆alkyl;

R^{e} is H, C₁₋₆alkyl, Ar—C₀₋₆alkyl, Het-C₀₋₆alkyl, C₃₋₇cycloalkyl-C₀₋₆alkyl, or (CH₂)ₖCO₂R^{g};

R^{b} and R^{c} are independently selected from H, C₁₋₆alkyl, Ar—C₀₋₆alkyl, Het-C₀₋₆alkyl, or C₃₋₆cycloalkyl-C₀₋₆alkyl, halogen, CF₃, OR^{f}, S(O)ₖR^{f}, COR^{f}, NO₂, N(R^{f})₂, CO(NR^{f})₂, CH₂N(R^{f})₂, or R^{b} and R^{c} are joined together to form a five or six membered aromatic or non-aromatic carbocyclic or heterocyclic ring, optionally substituted by up to three substituents chosen from halogen, CF₃, C₁₋₄alkyl, OR^{f}, S(O)ₖR^{f}, COR^{f}, CO₂R^{f}, OH, NO₂, N(R^{f})₂, CO(NR^{f})₂, and CH₂N(R^{f})₂; or methylenedioxy;

Q¹, Q², Q³ and Q⁴ are independently N or C—R^{y}, provided that no more than one of Q¹, Q², Q³ and Q₄ is N;

R′ is H, C₁₋₆alkyl, Ar—C₀₋₆alkyl or C₃₋₆cycloalkyl-C₀₋₆alkyl;

R″ is R′, —C(O)R′ or —C(O)OR′;

R^{y} is H, halo, —OR^{g}, —SR^{g}, —CN, —NR^{g}R^{k}, —NO₂, —CF₃, CF₃S(O)ᵣ—, —CO₂R^{g}, —COR^{g} or —CONR^{g}₂, or C₁₋₆alkyl optionally substituted by halo, —OR^{g}, —SR^{g}, —CN, NR^{g}R″, —NO₂, —CF₃, R′S(O)ᵣ—, —CO₂R^{g}, —COR^{g} or —CONR^{g}₂;

a is 0, 1 or 2;

b is 0, 1 or 2;

k is 0, 1 or 2;

r is 0, 1 or 2;

s is 0, 1 or 2;

u is 0 or 1; and

v is 0 or 1;

or a pharmaceutically acceptable salt thereof.

Suitably, this invention comprises formula (I) compounds of formula (Ia):

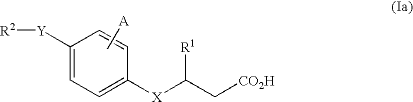

wherein:

X is CR′R′, NR′, O or S;

Y is CR′R′, NR′, O or S;

A is H, halo, —OR^{g}, —SR^{g}, —CN, —NR^{g}R^{k}, —NO₂, —CF₃, —S(O)ᵣCF₃, —CO₂R^{g}, —COR^{g}, —CONR^{g}₂—C₁₋₆alkyl, —C₀₋₆alkyl-Ar, —C₀₋₆alkyl-Het, —C₀₋₆alkyl-C₃₋₆cycloalkyl, —S(O)ₖR^{g}, or CH₂N(R^{f})₂;

R¹ is —C₀₋₆alkyl-Het-, —C₀₋₆alkyl-Ar, H, —CN or —S(O)ₖR^{g};

R² is

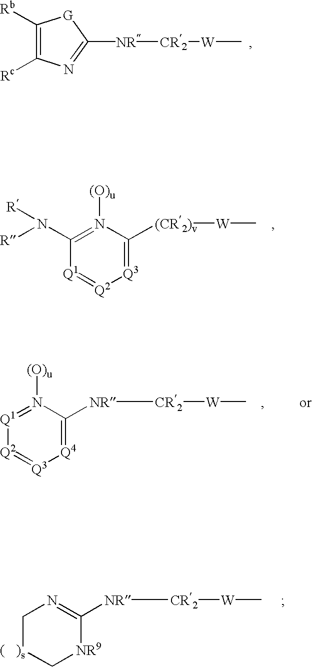

W is —(CHR^{g})ₐ—U—(CHR^{g})_{b}—;

U is absent or CO, CR^{g}₂, C(═CR^{g}₂), S(O)ₖ, O, NR^{g}, CR^{g}OR^{g}, CR^{g}(OR^{k})CR^{g}₂, CR^{g}₂CR^{g}(OR^{k}), C(O)CR^{g}₂, CR^{g}₂C(O), CONRⁱ, NRⁱCO, OC(O), C(O)O, C(S)O, OC(S), C(S)NR^{g}, NR^{g}C(S), S(O)₂NR^{g}, NR^{g}S(O)₂ N═N, NR^{g}NR^{g}, NR^{g}CR^{g}₂, CR^{g}₂NR^{g}, CR^{g}₂O, OCR^{g}₂, C≡C , CR^{g}═CR^{g}, Ar or Het;

G is NR^{e}, S or O;

R^{g} is H, C₁₋₆alkyl, Het-C₀₋₆alkyl, C₃₋₇cycloalkyl-C₀₋₆alkyl or Ar—C₀₋₆alkyl;

R^{k} is R^{g}, —C(O)R^{g}, or —C(O)OR^{f};

Rⁱ is is H, C₁₋₆alkyl, Het-C₀₋₆alkyl, C₃₋₇cycloalkyl-C₀₋₆alkyl, Ar—C₀₋₆alkyl, or C₁₋₆alkyl substituted by one to three groups chosen from halogen, CN, NR^{g}₂, OR^{g}, SR^{g}, CO₂R^{g}, and CON(R^{g})₂;

R^{f} is H, C₁₋₆alkyl or Ar—C₀₋₆alkyl;

R^{e} is H, C₁₋₆alkyl, Ar—C₀₋₆alkyl, Het-C₀₋₆alkyl, C₃₋₇cycloalkyl-C₀₋₆alkyl, or (CH₂)ₖCO₂R^{g};

R^{b} and R^{c} are independently selected from H, C₁₋₆alkyl, Ar—C₀₋₆alkyl, Het-C₀₋₆alkyl, or C₃₋₆cycloalkyl-C₀₋₆alkyl, halogen, CF₃, OR^{f}, S(O)ₖR^{f}, COR^{f}, NO₂, N(R^{f})₂, CO(NR^{f})₂, CH₂N(R^{f})₂, or R^{b} and R^{c} are joined together to form a five or six membered aromatic or non-aromatic carbocyclic or heterocyclic ring, optionally substituted by up to three substituents chosen from halogen, CF₃, C₁₋₄alkyl, OR^{f}, S(O)ₖR^{f}, COR^{f}, CO₂R^{f}, OH, NO₂, N(R^{f})₂, CO(NR^{f})₂, and CH₂N(R^{f})₂; or methylenedioxy;

Q¹, Q², Q³ and Q⁴ are independently N or C—R^{y}, provided that no more than one of Q¹, Q², Q³ and Q⁴ is N;

R′ is H, C₁₋₆alkyl, Ar—C₀₋₆alkyl or C₃₋₆cycloalkyl-C₀₋₆alkyl;

R″ is R′, —C(O)R′ or —C(O)OR′;

R^{y} is H, halo, —OR^{g}, —SR^{g}, —CN, —NR^{g}R^{k}, —NO₂, —CF₃, CF₃S(O)ᵣ—, —CO₂R^{g}, —COR^{g} or —CONR^{g}₂, or C₁₋₆alkyl optionally substituted by halo, —OR^{g}, —SR^{g}, —CN, —NR^{g}R″, —NO₂, —CF₃, R′S(O)ᵣ—, —CO₂R^{g}, —COR^{g} or —CONR^{g}₂;

a is 0, 1 or 2;

b is 0, 1 or 2;

k is 0, 1 or 2;

r is 0, 1 or 2;

s is 0, 1 or 2;

u is 0 or 1; and

v is 0 or 1;

or a pharmaceutically acceptable salt thereof.

Also included in this invention are pharmaceutically acceptable addition salts and complexes of the compounds of this invention. In cases wherein the compounds of this invention may have one or more chiral centers, unless specified, this invention includes each unique nonracemic compound which may be synthesized and resolved by conventional techniques. In cases in which compounds have unsaturated carbon-carbon double bonds, both the cis (Z) and trans (E) isomers are within the scope of this invention. In cases wherein compounds may exist in tautomeric forms, such as keto-enol tautomers, such as

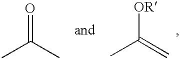

and each tautomeric form is contemplated as being included within this invention whether existing in equilibrium or locked in one form by appropriate substitution with R′.

The compounds of formula (I) inhibit the binding of vitronectin and other RGD-containing peptides to the vitronectin receptor. Inhibition of the vitronectin receptor on osteoclasts inhibits osteoclastic bone resorption and is useful in the treatment of diseases wherein bone resorption is associated with pathology, such as osteoporosis and osteoarthritis.

In another aspect, this invention is a method for stimulating bone formation which comprises administering a compound which causes an increase in osteocalcin release. Increased bone production is a clear benefit in disease states wherein there is a deficiency of mineralized bone mass or remodeling of bone is desired, such as fracture healing and the prevention of bone fractures. Diseases and metabolic disorders which result in loss of bone structure would also benefit from such treatment. For instance, hyperparathyroidism, Paget's disease, hypercalcemia of malignancy, osteolytic lesions produced by bone metastasis, bone loss due to immobilization or sex hormone deficiency, Behcet's disease, osteomalacia, hyperostosis and osteopetrosis, could benefit from administering a compound of this invention.

Additionally. since the compounds of the instant invention inhibit vitronectin receptors on a number of different types of cells, said compounds would be useful in the treatment of inflammatory disorders, such as rheumatoid arthritis and psoriasis, and cardiovascular diseases, such as atherosclerosis and restenosis. The compounds of Formula (I) of the present invention may be useful for the treatment or prevention of other diseases including, but not limited to, thromboembolic disorders, asthma, allergies, adult respiratory distress syndrome, graft versus host disease, organ transplant rejection, septic shock, eczema, contact dermatitis, inflammatory bowel disease, and other autoimmune diseases. The compounds of the present invention may also be useful for wound healing.

The compounds of the present invention are also useful for the treatment, including prevention, of angiogenic disorders. The term angiogenic disorders as used herein includes conditions involving abnormal neovascularization. Where the growth of new blood vessels is the cause of, or contributes to, the pathology associated with a disease, inhibition of angiogenisis will reduce the deleterious effects of the disease. An example of such a disease target is diabetic retinopathy. Where the growth of new blood vessels is required to support growth of a deleterious tissue, inhibition of angiogenisis will reduce the blood supply to the tissue and thereby contribute to reduction in tissue mass based on blood supply requirements. Examples include growth of tumors where neovascularization is a continual requirement in order that the tumor grow and the establishment of solid tumor metastases. Thus, the compounds of the present invention inhibit tumor tissue angiogenesis, thereby preventing tumor metastasis and tumor growth.

Thus, according to the methods of the present invention, the inhibition of angiogenesis using the compounds of the present invention can ameliorate the symptoms of the disease, and, in some cases, can cure the disease.

Another therapeutic target for the compounds of the instant invention are eye diseases chacterized by neovascularization. Such eye diseases include corneal neovascular disorders, such as corneal transplantation, herpetic keratitis, luetic keratitis, pterygrum and neovascular pannus associated with contact lens use. Additional eye diseases also include age-related macular degeneration, presumed ocular histoplasmosis, retinopathy of prematurity and neovascular glaucoma.

This invention further provides a method of inhibiting tumor growth which comprises administering stepwise or in physical combination a compound of formula (I) and an antineoplastic agent, such as topotecan and cisplatin.

With respect to formula (I) and (Ia):

Suitably R² is

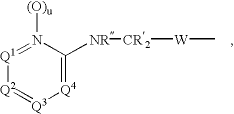

wherein Q¹, Q², and Q³ are each CR^{y}, Q⁴ is CR^{y} or N and u is 0, and preferably, each R′ is H, R″ is H or C₁₋₆alkyl, W is —(CH₂)₁₋₄—, Q⁴ is CR^{y} and R^{y} is H.

Alternately R² is

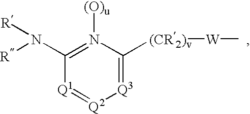

wherein Q¹, Q², and Q³ are each CH and u is 0, and preferably, each R′ is H, R″ is H or C₁₋₆alkyl, W is —CH₂—CH₂— and v is 0.

Alternately R² is

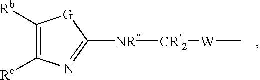

wherein G is NH and R^{b} and R^{c} are each H, and preferably, W is —CH₂—CH₂—.

Alternately R² is

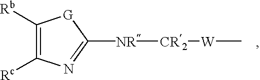

wherein G is NH and R^{b} and R^{c} are joined together to form a five or six membered aromatic or non-aromatic carbocyclic or heterocyclic ring, optionally substituted by up to three substituents chosen from halogen, CF₃, C₁₋₄alkyl, OR^{f}, S(O)ₖR^{f}, COR^{f}, CO₂R^{f}, OH, NO₂, N(R^{f})₂, CO(NR^{f})₂, and CH₂N(R^{f})₂; or methylenedioxy. Preferably, R^{b} and R^{c} are joined together to form a six membered aromatic carbocyclic or heterocyclic ring and W is —CH₂—CH₂—.

Alternately R² is

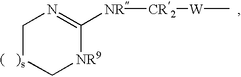

wherein each R′ is H, R″ is H or C₁₋₆alkyl, R^{g} is H or C₁₋₆alkyl and s is 0, 1 or 2 and, preferably, W is —CH₂—CH₂—.

Alternately, R² is

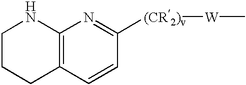

wherein v is 0 and W is —CH₂—CH₂—.

With respect to formula (I), suitably R¹ is is phenyl, benzyl, pyridyl, imidazolyl, oxazolyl or thiazolyl. Preferably, R¹ is phenyl. Suitably, Y is O or CH₂ and X is NH or CH₂. Preferably, Y is O.

Representative of the novel compounds of this invention are the compounds named in Examples 1-43 hereinafter.

In cases wherein the compounds of this invention may have one or more chiral centers, unless specified, this invention includes each unique nonracemic compound which may be synthesized and resolved by conventional techniques. According to the present invention, the (S) configuration of the formula (I) compounds is preferred.

In cases in which compounds have unsaturated carbon-carbon double bonds, both the cis (Z) and trans (E) isomers are within the scope of this invention. The meaning of any substituent at any one occurrence is independent of its meaning, or any other substituent's meaning, at any other occurrence.

Also included in this invention are prodrugs of the compounds of this invention. Prodrugs are considered to be any covalently bonded carriers which release the active parent drug according to formula (I) in vivo. Thus, in another aspect of this invention are novel prodrugs, which are also intermediates in the preparation of formula (Ia) compounds, of formula (II):

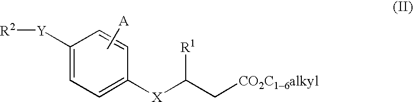

wherein:

X is CR′R′, NR′, O or S;

Y is CR′R′, NR′, O or S;

A is H, halo, —OR^{g}, —SR^{g}, —CN, —NR^{g}R^{k}, —NO₂, —CF₃, —S(O)ᵣCF₃, —CO₂R^{g}, —COR^{g}, —CONR^{g}₂—C₁₋₆alkyl, —C₀₋₆alkyl-Ar, —C₀₋₆alkyl-Het, —C₀₋₆alkyl-C₃₋₆cycloalkyl, —S(O)ₖR^{g}, or —CH₂N(R^{f})₂;

R¹ is —C₀₋₆alkyl-Het-, —C₀₋₆alkyl-Ar, H, —CN or —S(O)ₖR^{g};

R² is

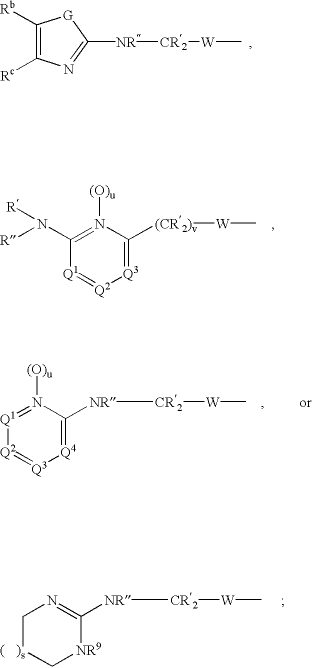

W is —(CHR^{g})ₐ—U—(CHR^{g})_{b}—;

U is absent or CO, CR^{g}₂, C(═CR^{g}₂), S(O)ₖ, O. NR^{g}, CR^{g}OR^{g}, CR^{g}(OR^{k})CR^{g}₂, CR^{g}₂CR^{g}(OR^{k}), C(O)CR^{g}₂, CR^{g}₂C(O), CONRⁱ, NRⁱCO, OC(O), C(O)O, C(S)O, OC(S), C(S)NR^{g}, NR^{g}C(S), S(O)₂NR^{g}, NR^{g}S(O)₂ N═N, NR^{g}NR^{g}, NR^{g}CR^{g}₂, CR^{g}₂NR^{g}, CR^{g}₂O, OCR^{g}₂, C≡C, CR^{g}═CR^{g}, Ar or Het;

G is NR^{e}, S or O;

R^{g} is H, C₁₋₆alkyl, Het-C₀₋₆alkyl, C₃₋₇cycloalkyl-C₀₋₆alkyl or Ar—C₀₋₆alkyl;

R^{k} is R^{g}, C(O)R^{g}, or —C(O)OR^{f};

Rⁱ is is H, C₁₋₆alkyl, Het-C₀₋₆alkyl, C₃₋₇cycloalkyl-C₀₋₆alkyl, Ar—C₀₋₆alkyl, or C₁₋₆alkyl substituted by one to three groups chosen from halogen, CN, NR^{g}₂, OR^{g}, SR^{g}, CO₂R^{g}, and CON(R^{g})₂;

R^{f} is H, C₁₋₆alkyl or Ar—C₀₋₆alkyl;

R^{e} is H, C₁₋₆alkyl, Ar—C₀₋₆alkyl, Het-C₀₋₆alkyl, C₃₋₇cycloalkyl-C₀₋₆alkyl, or (CH₂)ₖCO₂R^{g};

R^{b} and R^{c} are independently selected from H, C₁₋₆alkyl, Ar—C₀₋₆alkyl, Het-C₀₋₆alkyl, or C₃₋₆cycloalkyl-C₀₋₆alkyl, halogen, CF₃, OR^{f}, S(O)ₖR^{f}, COR^{f}, NO₂, N(R^{f})₂, CO(NR^{f})₂, CH₂N(R^{f})₂, or R^{b} and R^{c} are joined together to form a five or six membered aromatic or non-aromatic carbocyclic or heterocyclic ring, optionally substituted by up to three substituents chosen from halogen, CF₃, C₁₋₄alkyl, OR^{f}, S(O)ₖR^{f}, COR^{f}, CO₂R^{f}, OH, NO₂, N(R^{f})₂, CO(NR^{f})₂, and CH₂N(R^{f})₂; or methylenedioxy;

Q¹, Q², Q³ and Q⁴ are independently N or C—R^{y}, provided that no more than one of Q¹, Q², Q³ and Q⁴ is N;

R′ is H, C₁₋₆alkyl, Ar—C₀₋₆alkyl or C₃₋₆cycloalkyl-C₀₋₆alkyl;

R″ is R′, —C(O)R′ or —C(O)OR′;

R^{y} is H, halo, —OR^{g}, —SR^{g}, —CN, —NR^{g}R^{k}, —NO₂, —CF₃, CF₃S(O)ᵣ—, —CO₂R^{g}, —COR^{g} or —CONR^{g}₂, or C₁₋₆alkyl optionally substituted by halo, —OR^{g}, —SR^{g}, —CN, —NR^{g}R″, —NO₂, —CF₃, R′S(O)ᵣ—, —CO₂R^{g}, —COR^{g} or —CONR^{g}₂;

a is 0, 1 or 2;

b is 0, 1 or 2;

k is 0, 1 or 2;

r is 0, 1 or 2;

s is 0, 1 or 2;

u is 0 or 1; and

v is 0 or 1;

or a pharmaceutically acceptable salt thereof.

In yet another aspect of this invention are novel intermediates of formula (III):

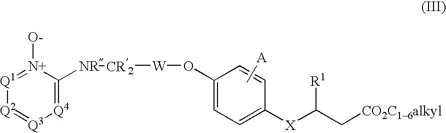

wherein:

X is CR′R′, NR′, O or S;

Y is CR′R′, NR′, O or S;

A is H, halo, —OR^{g}, —SR^{g}, —CN, —NR^{g}R^{k}, —NO₂, —CF₃, —S(O)ᵣCF₃, —CO₂R^{g}, —COR^{g}, —CONR^{g}₂—C₁₋₆alkyl, —C₀₋₆alkyl-Ar, —C₀₋₆alkyl-Het, —C₀₋₆alkyl-C₃₋₆cycloalkyl, —S(O)ₖR^{g}, or CH₂N(R^{f})₂;

R¹ is —C₀₋₆alkyl-Het-, —C₀₋₆alkyl-Ar, H, —CN or —S(O)ₖR^{g};

W is —(CHR^{g})ₐ—U—(CHR^{g})_{b}—;

U is absent or CO, CR^{g}₂, C(═CR^{g}₂), S(O)ₖ, O, NR^{g}, CR^{g}OR^{g}, CR^{g}(OR^{k})CR^{g}₂, CR^{g}₂CR^{g}(OR^{k}), C(O)CR^{g}₂, CR^{g}₂C(O), CONRⁱ, NRⁱCO, OC(O), C(O)O, C(S)O, OC(S), C(S)NR^{g}, NR^{g}C(S), S(O)₂NR^{g}, NR^{g}S(O)₂ N═N, NR^{g}NR^{g}, NR^{g}CR^{g}₂, CR^{g}₂NR^{g}, CR^{g}₂O, OCR^{g}₂, C≡C, CR^{g}═CR^{g}, Ar or Het;

R^{g} is H, C₁₋₆alkyl, Het-C₀₋₆alkyl, C₃₋₇cycloalkyl-C₀₋₆alkyl or Ar—C₀₋₆alkyl;

R^{k} is R^{g}, —C(O)R^{g}, or —C(O)OR^{f};

Rⁱ is is H, C₁₋₆alkyl, Het-C₀₋₆alkyl, C₃₋₇cycloalkyl-C₀₋₆alkyl, Ar—C₀₋₆alkyl, or C₁₋₆alkyl substituted by one to three groups chosen from halogen, CN, NR^{g}₂, OR^{g}, SR^{g}, CO₂R^{g}, and CON(R^{g})₂;

R^{f} is H, C₁₋₆alkyl or Ar—C₀₋₆alkyl;

Q¹, Q², Q³ and Q⁴ are independently N or C—R^{y}, provided that no more than one of Q¹, Q², Q³ and Q⁴ is N;

R′ is H, C₁₋₆alkyl, Ar—C₀₋₆alkyl or C₃₋₆cycloalkyl-C₀₋₆alkyl;

R″ is R′, —C(O)R′ or —C(O)OR′;

R^{y} is H, halo, —OR^{g}, —SR^{g}, —CN, —NR^{g}R^{k}, —NO₂, —CF₃, CF₃S(O)ᵣ—, —CO₂R^{g}, —COR^{g} or —CONR^{g}₂, or C₁₋₆alkyl optionally substituted by halo, —OR^{g}, —SR^{g}, —CN, —NR^{g}R″, —NO₂, —CF₃, R′S(O)ᵣ—, —CO₂R^{g}, —COR^{g} or —CONR^{g}₂;

a is 0, 1 or 2; and

b is 0, 1 or 2;

or a pharmaceutically acceptable salt thereof.

Abbreviations and symbols commonly used in the peptide and chemical arts are used herein to describe the compounds of this invention. In general, the amino acid abbreviations follow the IUPAC-IUB Joint Commission on Biochemical Nomenclature as described in Eur. J. Biochem., 158, 9 (1984).

C₁₋₄alkyl as applied herein means an optionally substituted alkyl group of 1 to 4 carbon atoms, and includes methyl, ethyl, n-propyl, isopropyl, n-butyl, isobutyl and t-butyl. C₁₋₆alkyl additionally includes pentyl, n-pentyl, isopentyl, neopentyl and hexyl and the simple aliphatic isomers thereof. C₀₋₄alkyl and C₀₋₆alkyl additionally indicates that no alkyl group need be present (e.g., that a covalent bond is present).

Any C₁₋₄alkyl or C₁₋₆ alkyl, C₂₋₆ alkenyl, C₂₋₆ alkynyl or C₁₋₆ oxoalkyl may be optionally substituted with the group R^{X}, which may be on any carbon atom that results in a stable structure and is available by conventional synthetic techniques. Suitable groups for R^{X} are C₁₋₄alkyl, OR′, SR′, C₁₋₄alkylsulfonyl, C₁₋₄alkylsulfoxyl, —CN, N(R′)₂, CH₂N(R′)₂, —NO₂, —CF₃, —CO₂R′—CON(R′)₂, —COR′, —SO₂N(R′)₂, —NR′C(O)R′, F, Cl, Br, I, or CF₃S(O)ᵣ—, wherein r is 0, 1 or 2.

Halogen or halo means F, Cl, Br, and I.

Ar, or aryl, as applied herein, means phenyl or naphthyl. or phenyl or naphthyl substituted by one to three substituents, such as those defined above for alkyl, especially C₁₋₄alkyl, C₁₋₄alkoxy, C₁₋₄alkthio, CF₃, NH₂, OH, F, Cl, Br or I.

Het, or heterocycle, indicates an optionally substituted five or six membered monocyclic ring, or a nine or ten-membered bicyclic ring containing one to three heteroatoms chosen from the group of nitrogen, oxygen and sulfur, which are stable and available by conventional chemical synthesis. Illustrative heterocycles are benzofuran, benzimidazole, benzopyran, benzothiophene, benzothiazole, furan, imidazole, indoline, morpholine, piperidine, piperazine, pyrrole, pyrrolidine, tetrahydropyridine, pyridine, thiazole, oxazole, thiophene, quinoline, isoquinoline. and tetra- and perhydro-quinoline and isoquinoline. Any accessible combination of up to three substituents on the Het ring, such as those defined above for alkyl that are available by chemical synthesis and are stable are within the scope of this invention.

C₃₋₇cycloalkyl refers to an optionally substituted carbocyclic system of three to seven carbon atoms, which may contain up to two unsaturated carbon-carbon bonds. Typical of C₃₋₇cycloalkyl are cyclopropyl, cyclobutyl, cyclopentyl, cyclopentenyl, cyclohexyl, cyclohexenyl and cycloheptyl. Any combination of up to three substituents, such as those defined above for alkyl, on the cycloalkyl ring that is available by conventional chemical synthesis and is stable, is within the scope of this invention.

When R^{b} and R^{c} are joined together to form a five- or six-membered aromatic or non-aromatic carbocyclic or heterocyclic ring fused to the ring to which R^{b} and R^{c} are attached, the ring formed will generally be a five- or six-membered heterocycle selected from those listed above for Het, or will be a phenyl, cyclohexyl or cyclopentyl ring. Preferably R_{b} and R_{c} will be —D1═D2—D3═D4 wherein D1—D4 are independently CH, N or C—R_{X} with the proviso that no more than two of D1—D4 are N. Most preferably, when R^{b} and R^{c} are joined together they form the group —CH═CH—CH═CH—.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical, Bn refers to the benzyl radical, Me refers to methyl, Et refers to ethyl, Ac refers to acetyl, Alk refers to C₁₋₄alkyl, Nph refers to 1- or 2-naphthyl and cHex refers to cyclohexyl. Tet refers to 5-tetrazolyl.

Certain reagents are abbreviated herein. DCC refers to dicyclohexylcarbodiimide. DMAP refers to dimethylaminopyridine, DIEA refers to diisopropylethyl amine, EDC refers to 1-(3-dimethylaminopropyl)-3-ethylcarbodiimide, hydrochloride. HOBt refers to 1-hydroxybenzotriazole, THF refers to tetrahydrofuran, DIEA refers to diisopropylethylamine, DEAD refers to diethyl azodicarboxylate, PPh₃ refers to triphenylphosphine, DIAD refers to diisopropyl azodicarboxylate, DME refers to dimethoxyethane, DMF refers to dimethylformamide, NBS refers to N-bromosuccinimide, Pd/C refers to a palladium on carbon catalyst, PPA refers to polyphosphoric acid, DPPA refers to diphenylphosphoryl azide, BOP refers to benzotriazol-1-yloxy-tris(dimethyl-amino)phosphonium hexafluorophosphate, HF refers to hydrofluoric acid, TEA refers to triethylamine, TFA refers to trifluoroacetic acid, PCC refers to pyridinium chlorochromate.

The compounds of formula (Ia) are generally prepared by reacting a compound of formula (IV) with a compound of formula (V):

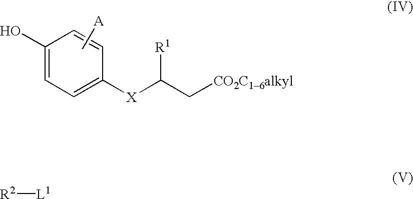

wherein R¹, R², A and X are as defined in formula (Ia), with any reactive functional groups protected, and L¹ is OH or halo;

and thereafter removing any protecting groups, and optionally forming a pharmaceutically acceptable salt.

Suitably, certain compounds of formula (Ia) are prepared by reacting a compound of formula (IV), as defined hereinbefore, with a compound of formula (VI):

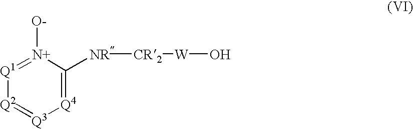

wherein R′, R″, W, Q¹, Q², Q³ and Q⁴ are as defined in formula (Ia), with any reactive functional groups protected;

and thereafter removing any protecting groups, and optionally forming a pharmaceutically acceptable salt.

Preferably, for formula (VI) compounds, Q¹, Q², Q³ and Q⁴ are CH, W is —(CH₂)₁₋₄—, R′ is H and R″ is H or C₁₋₆alkyl. Suitably, the reaction between a compound of formual (IV) with a compound of formula (VI) is carried out in the presence of diethyl azodicarboxylate and triphenylphosphine in an aprotic solvent.

Additionally, certain compounds of formula (Ia) are prepared by reacting a compound of formula (IV), as defined hereinbefore, with a compound of formula (VII):

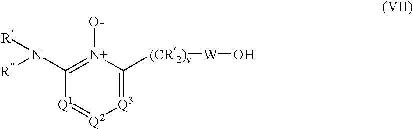

wherein R′, R″, W, Q¹, Q², Q³ and v are as defined in formula (Ia), with any reactive functional groups protected;

and thereafter removing any protecting groups, and optionally forming a pharmaceutically acceptable salt.

Preferably, for formula (VII) compounds, Q¹, Q² and Q³ are CH, W is —CH₂—CH₂—, R′ is H and R″ is H or C₁₋₆alkyl. Suitably, the reaction between a compound of formula (IV) with a compound of formula (VII) is carried out in the presence of diethyl azodicarboxylate and triphenylphosphine in an aprotic solvent.

Compounds of this invention, including formula (I) and (Ia) compounds, are prepared by the general methods described in Schemes I-XVI.

The preparation of compounds wherein Y is O and X is CH₂ is described in Scheme I.

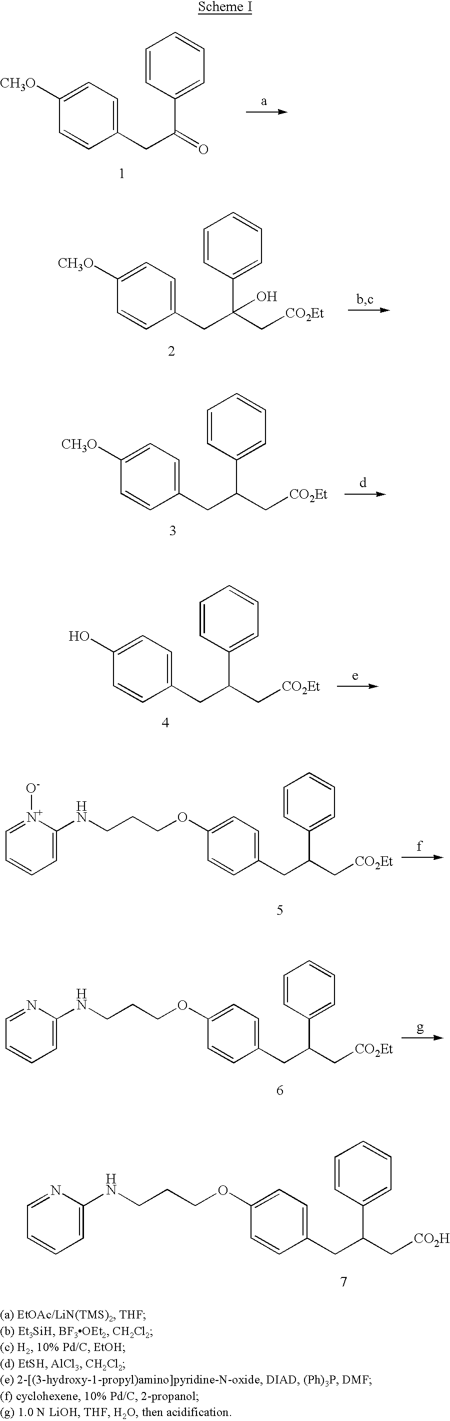

An appropriately substituted deoxybenzoin derivative, such as 2-(4-methoxyphenyl)-1-phenylethanone (Chem. Ber. 1958, 91, 755-759), is reacted in an aldol-type reaction with the enolate of ethyl acetate, which can be generated from ethyl acetate on exposure to an appropriate amide base, for instance lithium diisopropylamide (LDA) or lithium bis(trimethylsilyl)amide (LiN(TMS)₂), to afford I-2. Frequently, THF is the solvent of choice for an aldol reaction, although THF in the presence of various additives, for instance HMPA or TMEDA, is often used. Reaction of I-2 with triethylsilane (Et₃SiH) in the presence of boron trifluoride etherate (BF₃.OEt₂) according to the general protocol of Orphanopoulos and Smonu (Synth. Commun. 1988, 833) for the reduction of tertiary benzylic alcohols affords I-3, together with the olefinic product derived from β-elimination of the alcohol. The olefinic product can be conveniently converted to I-3 by hydrogenation over a palladium catalyst, such as palladium metal on activated carbon (Pd/C), in an appropriate inert solvent, for instance methanol, ethanol, or ethyl acetate. Removal of the methyl ether of I-3 to give I-4 can be accomplished by reaction with ethanethiol (EtSH) in the presence of a Lewis acid catalyst, preferably anhydrous aluminum trichloride (AlCl₃), in an inert solvent, for instance CH₂Cl₂. Other useful methods for removal of a methyl ether are described in Greene, “Protective Groups in Organic Synthesis” (published by Wiley-Interscience). Compound I-4 is reacted with 2-[(3-hydroxy-1-propyl)amino]pyridine-N-oxide in a Mitsunobu-type coupling reaction (Organic Reactions 1992, 42, 335-656; Synthesis 1981, 1-28) to afford I-5. The reaction is mediated by the complex formed between an azodicarboxylate diester, such as diethyl azodicarboxylate or diisopropyl azodicarboxylate, and triphenylphosphine, and is conducted in an aprotic solvent, for instance THF, CH₂Cl₂, or DMF. The pyridine-N-oxide moiety of I-5 is reduced to the corresponding pyridine I-6 under transfer hydrogenation conditions using a palladium catalyst, preferably palladium metal on activated carbon, in an inert solvent, for instance methanol, ethanol, or 2-propanol. Cyclohexene, 1,4cyclohexadiene, formic acid, and salts of formic acid, such as potassium formate or ammonium formate, are commonly used as the hydrogen transfer reagent in this type of reaction. The ethyl ester of I-6 is hydrolyzed using aqueous base, for example, LiOH in aqueous THF or NaOH in aqueous methanol or ethanol, and the intermediate carboxylate salt is acidified with a suitable acid, for instance TFA or HCl, to afford the carboxylic acid I-7. Alternatively, the intermediate carboxylate salt can be isolated, if desired, or a carboxylate salt of the free carboxylic acid can be prepared by methods well-known to those of skill in the art.

An alternative method for preraing formula (I) compounds is described in Scheme II.

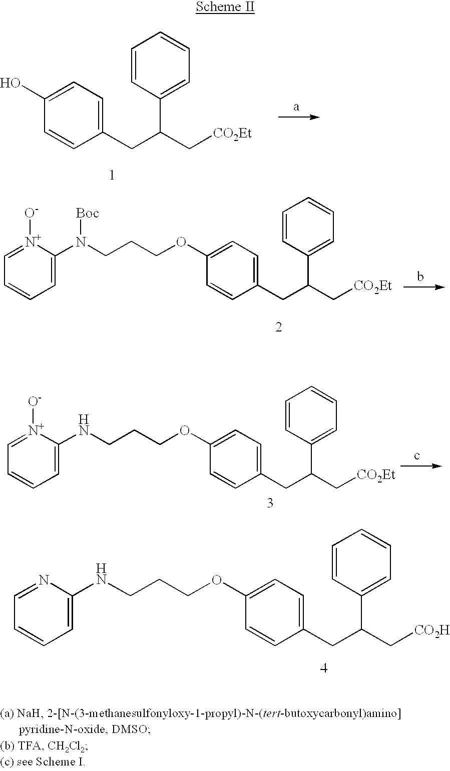

Compound II-1, prepared as described in Scheme I, is reacted with a base, suitably an alkali metal hydride such as sodium hydride or potassium hydride, in a polar, aprotic solvent, generally THF, DMF, DMSO, or mixtures thereof, to afford the corresponding alkali metal phenoxide. Alternatively, an alkali metal amide, for instance LDA, or the lithium, sodium, or potassium salt of hexamethyldisilazane, can be used for deprotonation. The intermediate phenoxide is generally not isolated, but is reacted in situ with an appropriate electrophile, for instance 2-[N-(3-methanesulfonyloxy-1-propyl)-N-(tert-butoxycarbonyl)amino]pyridine-N-oxide, to afford the coupled product II-2. The tert-butoxycarbonyl protecting group in II-2 is removed under acidic conditions, such as 4 M HCl in 1,4-dioxane or TFA in CH₂Cl₂, to afford II-3. Conditions for removal of the tert-butoxycarbonyl protecting group are well-known to those of skill in the art, and several useful methods are described in standard reference volumes such as Greene “Protective Groups in Organic Synthesis”. II-3 is subsequently converted to II-4 following the protocol outlined in Scheme I.

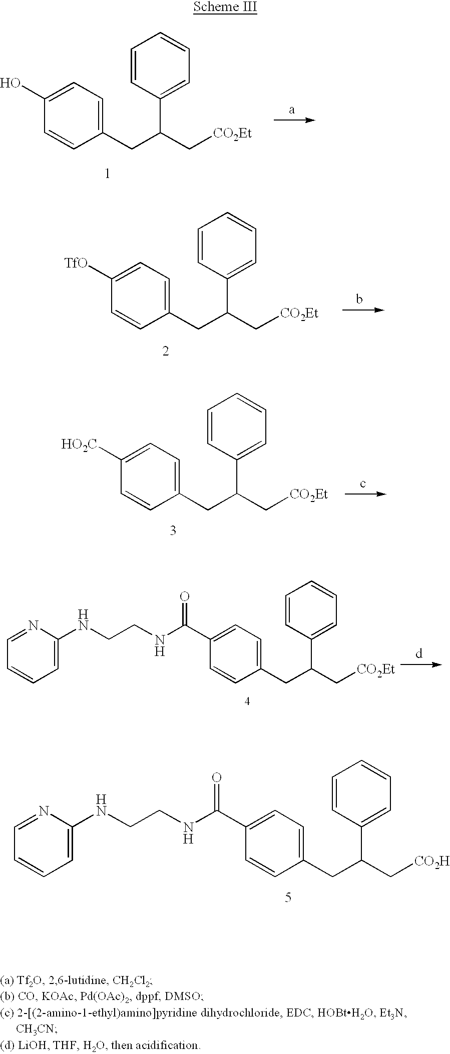

Phenol III-1, prepared as described in Scheme I, is converted to its trifluoromethanesulfonate ester III-2 by reaction with trifluoromethanesulfonic anhydride (Tf₂O) in the presence of a suitable non-nucleophilic amine base, such as 2,6-lutidine, in an inert solvent, generally CH₂Cl₂. III-2 reacts with carbon monoxide (CO) in the presence of potassium acetate, 1,1′-bis(diphenylphosphino)ferrocene (dppf), and a palladium catalyst, for instance palladium acetate (Pd(OAc)₂), in a suitable solvent, preferably DMSO, according to the general method described by Cacchi and Lupi (Tet. Lett. 1992, 33, 3939) for the carboxylation of aryl trifluoromethanesulfonates. The carboxylic acid of the resulting compound (III-3) is converted to an activated form using, for example, EDC and HOBt, or SOCl₂, and the activated form is subsequently reacted with an appropriate amine, for instance 2-[(2-amino-1-ethyl)amino]pyridine dihydrochloride, in a suitable solvent such as DMF, CH₂Cl₂, or CH₃CN, to afford III-4 Depending on whether acid neutralization is required, an added base, such as triethylamine (Et₃N), diisopropylethylamine ((i-Pr)₂NEt), or pyridine, may be used. Many additional methods for converting a carboxylic acid to an amide are known, and can be found in standard reference books, such as “Compendium of Organic Synthetic Methods”, Vol. I-VI (published by Wiley-Interscience), or Bodansky, “The Practice of Peptide Synthesis” (published by Springer-Verlag). The ethyl ester of III-4 is hydrolyzed using aqueous base, for example, LiOH in aqueous THF or NaOH in aqueous methanol or ethanol, and the intermediate carboxylate salt is acidified with a suitable acid, for instance TFA or HCl, to afford the carboxylic acid III-5Alternatively, the intermediate carboxylate salt can be isolated, if desired, or a carboxylate salt of the free carboxylic acid can be prepared by methods well-known to those of skill in the art.

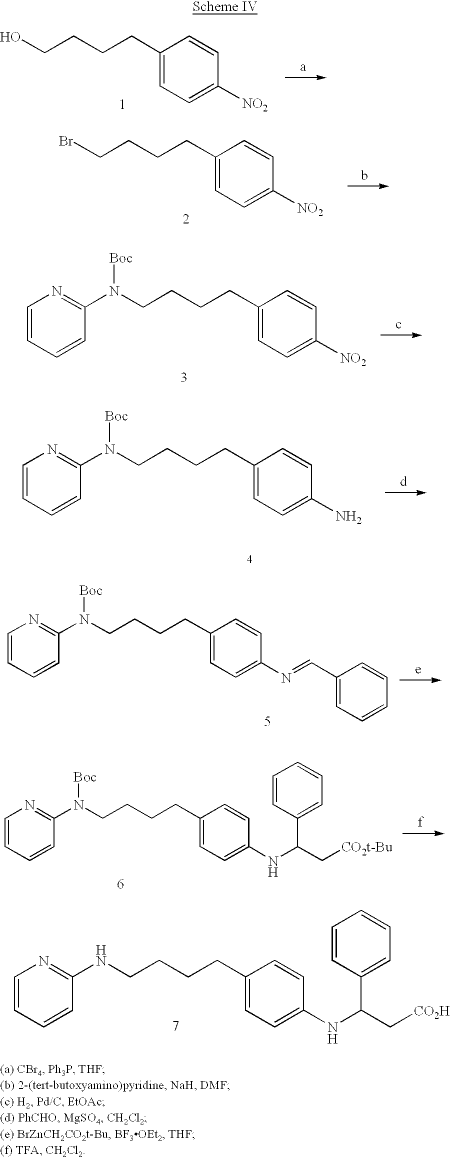

The commercially available alcohol IV-1 is converted to an activated species, for example the corresponding bromide IV-2, using carbon tetrabromide and triphenylphosphine in an inert solvent, preferably THF. Many other conditions are available for converting an alcohol to an activated species, such as the corresponding bromide, chloride, iodide, mesylate, or triflate, and are well-known to those of skill in the art. The bromide IV-2 is alkylated with a suitable 2-aminopyridine derivative, for instance 2-(tert-butoxyamino)pyridine, to afford the alkylated derivative IV-3. The reaction is mediated by an appropriate base, such as an alkali metal halide, and is conducted in a polar, aprotic solvent, generally THF, DMF, DMSO, or mixtures thereof. Reduction of the nitro group of IV-3 can be accomplished by a variety of methods well-known to those of skill in the art. Preferably, the reduction is accomplished by hydrogenation in the presence of a palladium catalyst, for instance palladium on activated charcoal, in a suitable solvent, such as EtOAc, MeOH, EtOH, i-PrOH, or mixtures thereof. The resulting aniline IV-4 reacts with a suitable aldehyde, such as benzaldehyde, in an inert solvent such as CH₂Cl₂, benzene, or toluene, to afford the corresponding aldimine IV-5. If desired, a dehydrating agent, such as MgSO₄, can be used to remove the H₂O formed during the reaction. The aldimine is subsequently reacted in an aldol-type reaction with an appropriate enolate of an acetic acid ester to afford IV-6. The reaction is generally mediated by a Lewis acid, for instance BF₃.OEt₂, and is usually conducted in an ethereal solvent, such as THF or DME. As described in Scheme I, the enolate can be generated from ethyl acetate on exposure to an appropriate amide base, for instance lithium diisopropylamide (LDA) or lithium bis(trimethylsilyl)amide (LiN(TMS)₂). Alternatively, the enolate can be generated from tert-butyl bromoacetate on exposure to zinc metal, according to the procedure of Orsoni and coworkers (Tetrahedron 1984, 40, 2781-2787). The tert-butoxycarbonyl group and the tert-butyl ester of IV-6 are removed simultaneously under acidic conditions, such as 4 M HCl in 1,4-dioxane or TFA in CH₂Cl₂, to afford IV-7. Conditions for deprotection of tert-butyl carbamates and tert-butyl esters are well-known to those of skill in the art, and several useful methods are described in standard reference volumes such as Greene “Protective Groups in Organic Synthesis” (published by Wiley-Interscience).

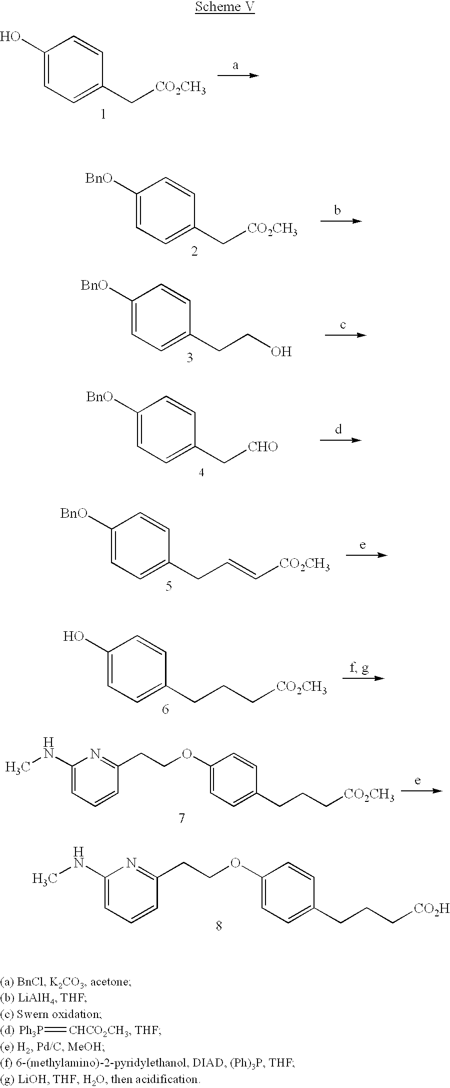

The phenol group of commercially available methyl 4-hydroxyphenylacetate (V-1) is protected with a suitable protecting group, for instance a methyl ether, a benzyl ether, or a triisopropylsilyl ether. Protection of phenols is well-known to those of skill in the art, and representative protecting groups are described in standard reference volumes such as Greene “Protective Groups in Organic Synthesis” (published by Wiley-Interscience). The ester group of V-2 is reduced to the corresponding primary alcohol using lithium aluminum hydride. Many other methods exist for the reduction of carboxylic acids and esters to alcohols, and are described in standard reference volumes, such as “Compendium of Organic Synthetic Methods” (published by Wiley-Interscience). The alcohol in V-3 is oxidized to the corresponding aldehyde using the well-know Swern conditions (J. Org. Chem. 1978, 43, 2480). Many other methods exist for the oxidation of alcohols to aldehydes, and are described in standard reference volumes, such as “Compendium of Organic Synthetic Methods” (published by Wiley-Interscience). Aldehyde V-4 is converted to the α,β-unsaturated ester V-5 through the well-known Wittig reaction. Optimally, the reaction is conducted using (carbomethoxymethylene)triphenylphosphorane in a polar, aprotic solvent, such as DMSO, THF, or mixtures thereof. Reduction of the olefin group of V-5 is optimally accomplished by hydrogenation in the presence of a palladium catalyst, for instance palladium on activated charcoal, in a suitable solvent, such as EtOAc, MeOH, EtOH, i-PrOH, or mixtures thereof. If a benzyl ether is used to protect the phenol group, it is simultaneously cleaved to liberate the free phenol. If another protecting group is used, suitable conditions are employed for its removal. For instance, if a methyl ether is used, it can be cleaved with ethanethiol (EtSH) and aluminum trichloride (AlCl₃) as described in Scheme I, or with boron tribromide (BBr₃), in an inert solvent, preferably CH₂Cl₂. Alternatively, if a triisopropylsilyl group is used, it can be cleaved using, for example, tetrabutylammonium fluoride, in a neutral solvent such as THF. Other useful methods for removal of phenolic protecting groups are described in Greene, “Protective Groups in Organic Synthesis” (published by Wiley-Interscience). The resulting phenol V-6 is reacted with 6-(methylamino)-2-pyridylethanol in a Mitsunobu-type coupling reaction (Organic Reactions 1992, 42, 335-656; Synthesis 1981, 1-28) to afford V-7. The reaction is mediated by the complex formed between an azodicarboxylate diester, such as diethyl azodicarboxylate or diisopropyl azodicarboxylate, and triphenylphosphine, and is conducted in an aprotic solvent, for instance THF, CH₂Cl₂, or DMF. V-7 is subsequently converted to V-8 according to the protocol described in Scheme III.

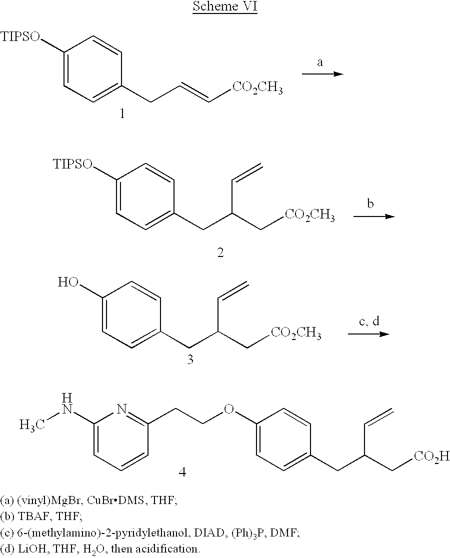

The α,β-unsaturated ester VI-1, prepared as described in Scheme V, is reacted with a cuprate reagent to effect a conjugate addition reaction. For example, reaction of VI-1 with the cuprate reagent derived from vinylmagnesium bromide and copper (I) bromide-dimethylsulfide complex, in an aprotic solvent such as Et₂O or THF, gives the conjugate addition product VI-2. Many procedures have been reported for the formation and conjugate addition reactions of a wide array of cuprate and organocopper reagents, and several excellent reviews have been published (for example, see Posner, Organic Reactions 1972, 19, 1-113; Lipshutz and Sengupta, Organic Reactions 1992, 41, 135-631). The triisopropylsilyl group of VI-2 is removed as described in Scheme V, and the resulting phenol VI-3 is converted to VI-4 according the methods described in Scheme V.

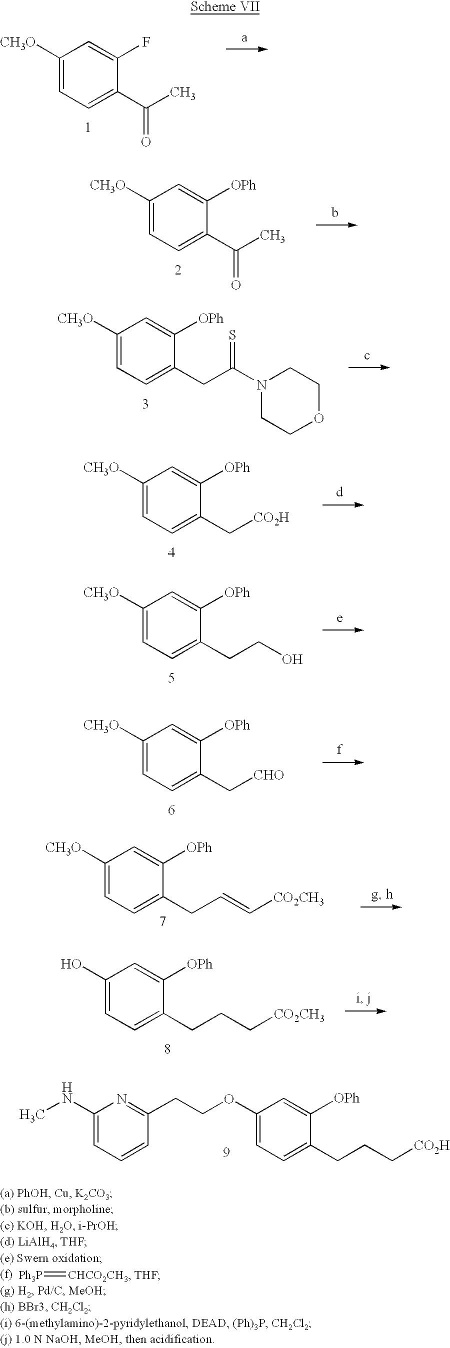

Commercially available 2-fluoro-4methoxyacetophenone (VII-1) reacts with an alcohol, for example phenol, in the presence of copper metal and a suitable base, for instance K₂CO₃, to afford the diaryl ether VII-2. On treatment with sulfur and an appropriate primary or secondary amine, preferably morpholine, according to the general method of Harris (J. Med. Chem. 1982, 25, 855), VII-2 is converted to VII-3 in a classical Willgerodt-Kindler reaction. The thioamide thus obtained is hydrolyzed to the corresponding carboxylic acid VII-4 by reaction with an alkali metal hydroxide, suitably KOH, in an aqueous alcoholic solvent, such as aqueous MeOH, EtOH, or i-PrOH. VII-4 is subsequently converted to VII-9 according to the general protocol described in Scheme V.

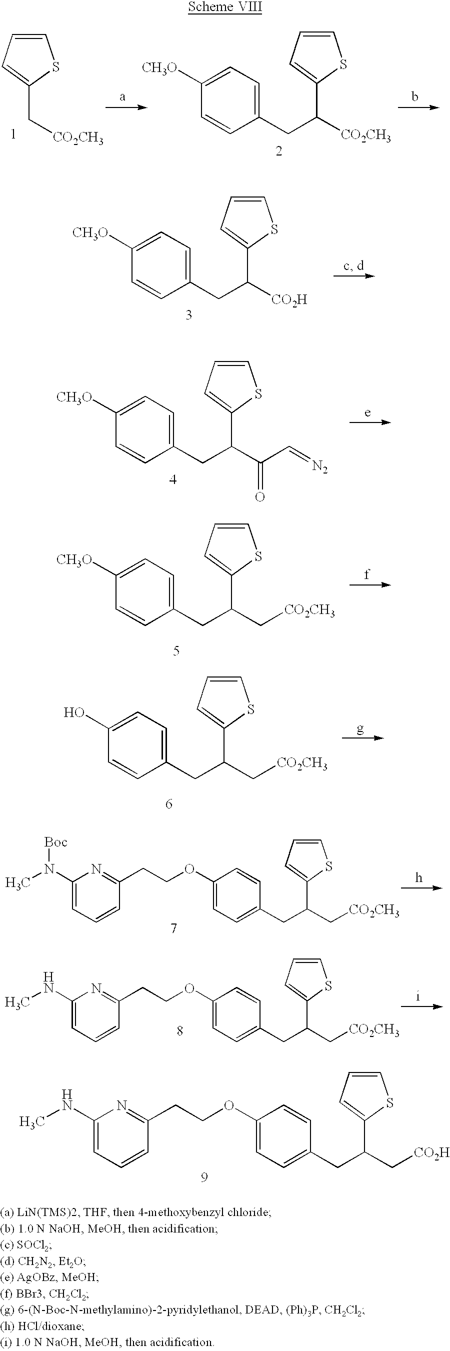

2-Thiopheneacetic acid methyl ester (VIII-1) is deprotonated with a suitable base, generally an alkali metal amide such as LDA or lithium bis(trimethylsilyl)amide, and without isolation the intermediate ester enolate is reacted with an appropriate benzyl halide, for instance 4-methoxybenzyl chloride, to afford the alkylation product VIII-2. Generally, a polar aprotic solvent such as THF, or THF in the presence of various additives, for instance HMPA or TMEDA, is preferred for this reaction. The methyl ester of VIII-2 is hydrolyzed using aqueous base, for example, LiOH in aqueous THF or NaOH in aqueous MeOH or EtOH, and the intermediate carboxylate salt is acidified with a suitable acid, for instance TFA or HCl, to afford the carboxylic acid VIII-3. This is converted to an activated form of the carboxylic acid using, for example, SOCl₂, and the activated form is subsequently reacted with diazomethane in a suitable solvent, such as Et₂O or a mixture of Et₂O and CH₂Cl₂, to afford the diazoketone VIII-4. On treatment with a suitable silver salt, for instance silver benzoate or silver triflate. in an alcoholic solvent, generally MeOH or EtOH. VIII-4 undergoes a classical Arndt-Eistert reaction to afford the ester VIII-5. Deprotection of the methyl ether according to the general conditions described in Scheme V gives VIII-6, which is converted to VIII-7 by reaction with 6-(N-Boc-N-methylamino)-2-pyridylethanol in a Mitsunobu reaction according to the conditions described in Scheme V. The tert-butoxycarbonyl group of VIII-7 is removed under acidic conditions, such as 4 M HCl in 1,4-dioxane or TFA in CH₂Cl₂, to afford VIII-8. Conditions for deprotection of tert-butyl carbamates are well-known to those of skill in the art, and several useful methods are described in standard reference volumes such as Greene “Protective Groups in Organic Synthesis”. Saponification of the according to the general methods described in Scheme III affords VIII-9.

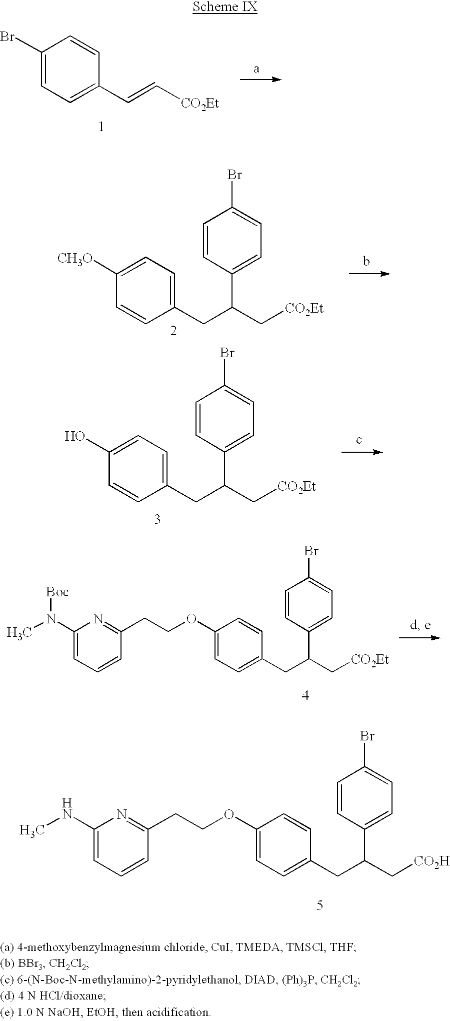

A suitable derivative of acrylic acid, for instance ethyl 4-bromocinnamate (IX-1), is converted to derivative IX-2 by reaction with selected benzyl cuprate reagents according to the general method of Van Heerden (Tetrahedron 1996, 52, 12313). As described in Scheme VI, many additional procedures have been reported for the formation and conjugate addition reactions of a wide array of cuprate and organocopper reagents. The addition product IX-2 is then converted to IX-5 by the general protocol described in Scheme VIII.

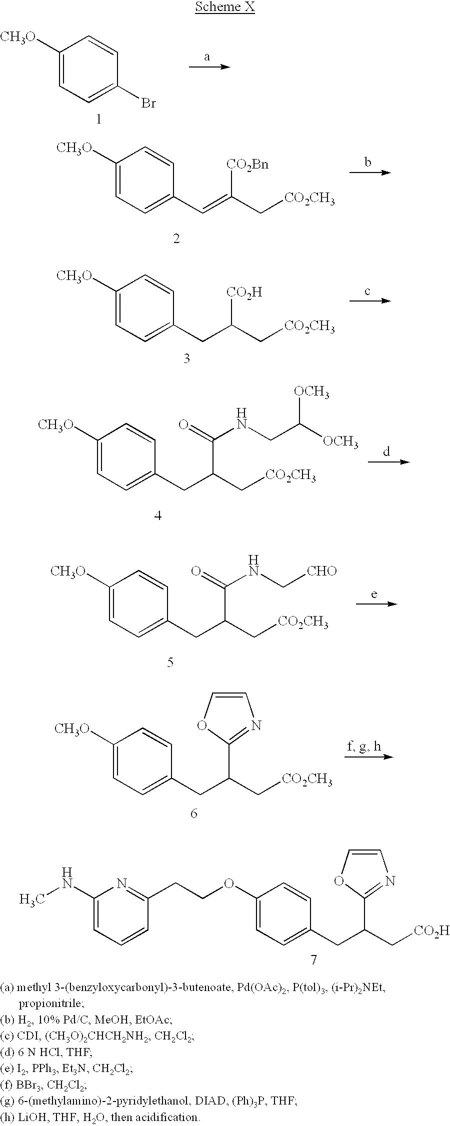

A suitable haloaromatic derivative, for instance 4-bromoanisole (X-1), reacts with methyl 3-(benzyloxycarbonyl)-3-butenoate in a Heck-type reaction (see Heck, Org. Reactions 1982, 27, 345) to afford X-2. The reaction is mediated by a palladium(0) species, and generally is conducted in an inert solvent, such as CH₃CN, propionitrile, or toluene, in the presence of an appropriate acid scavenger, such as triethylamine (Et₃N) or diisopropylethylamine ((i-Pr)₂NEt). Typical sources of the palladium(0) species include palladium (II) acetate (Pd(OAc)₂) and palladium(II) chloride (PdCl₂), and oftentimes phosphine ligands, for instance triphenylphosphine (PPh₃) or tri-ortho-tolylphosphine (P(tol)₃), are included. The α,β-unsaturated ester X-2 is reduced to the saturated compound X-3 by reaction with hydrogen gas in the presence of a suitable catalyst, preferably palladium metal on activated carbon (Pd/C), in an inert solvent, generally MeOH, EtOH, EtOAc, or mixtures thereof. The benzyl ester in X-2 is cleaved simultaneously under these conditions to liberate the corresponding carboxylic acid. The carboxylic acid of X-3 is converted to an activated form using, for example, EDC and HOBt, SOCl₂, or 1,1′-carbonyldiimidazole (CDI), and the activated form is subsequently reacted with an appropriate amine, for instance aminoacetaldehyde dimethyl acetal, in a suitable solvent, such as CH₂Cl₂, to afford X-4. Depending on whether acid neutralization is required, an added base, such as triethylamine (Et₃N), diisopropylethylamine ((i-Pr)₂NEt), or pyridine, may be used. Many additional methods for converting a carboxylic acid to an amide are known, and can be found in standard reference books, such as “Compendium of Organic Synthetic Methods”, Vol. I-VI (published by Wiley-Interscience), or Bodansky, “The Practice of Peptide Synthesis” (published by Springer-Verlag). The dimethyl acetal of X-4 is cleaved to the corresponding aidehyde (X-5) under acidic conditions, preferably with hydrochloric acid in THF or dioxane. Other methods for converting a dimethyl acetal to an aldehyde are described in standard reference volumes, such as Greene, “Protective Groups in Organic Synthesis” (published by Wiley-Interscience). The amidoaldehyde X-5 is cyclized to the oxazole X-6 according to the methodology of Rovnyak (J. Med. Chem. 1997, 40, 24-34). X-6 is then converted to X-7 according to the protocol described in Scheme V.

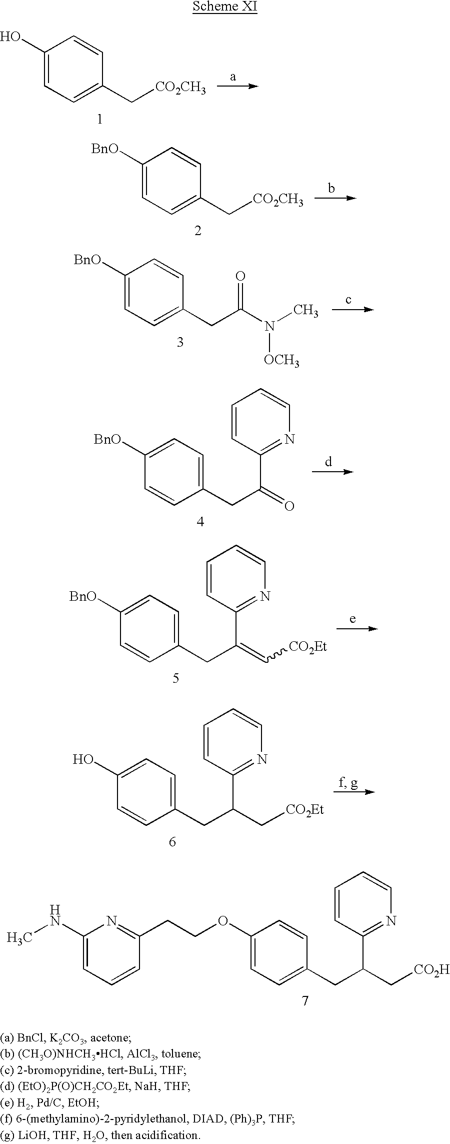

The phenol group of commercially available methyl 4-hydroxyphenylacetate (XI-1) is protected as its benzyl ether as described in Scheme V. The resulting compound (XI-2) reacts with N,O-dimethylhydroxylamine hydrochloride in the presence of AlCl₃ in an inert solvent, preferably toluene, according to the general method of Weinreb (Synth. Commun. 1982, 12, 989), to afford XI-3. This compound reacts with suitable Grignard or organolithium reagents to afford ketones according to the general procedure of Weinreb (Tet. Lett. 1981, 22, 3815). For example, 2-lithiopyridine, prepared from 2-bromopyrdine and tert-butyllithium, reacts with XI-3 in an ethereal solvent, such as THF or DME, to afford the ketone derivative XI-4. This ketone reacts in a Wittig-type reaction with triethyl phosphonoacetate in the presence of a suitable base, for instance LiN(TMS)2 or NaH, in a polar, aprotic solvent, preferably THF, to afford the α,β-unsaturated ester XI-5. As described in Scheme V, hydrogenation of XI-5 reduces the olefin and simultaneously removes the benzyl ether to afford XI-6. This compound is then converted to XI-7 by the protocol described in Scheme V.

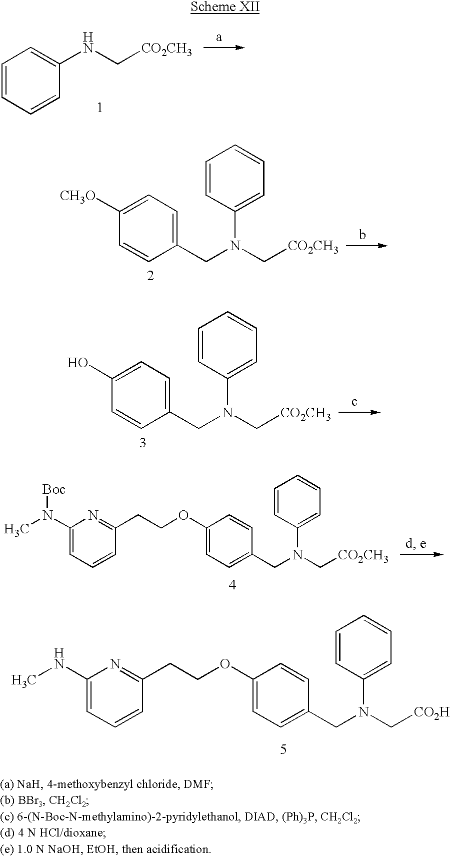

A suitably N-functionalized amino acid derivative, for instance N-phenylglycine (XII-1), is reacted with an appropriately functionalized benzyl halide, for example 4-methoxybenzyl chloride, to afford XII-2. The reaction is mediated by a base, such as NaH or LiN(TMS)2, and is conducted in a polar, aprotic solvent, generally THF, DMF, or mixtures thereof. The product XII-2 is subsequently converted to XII-5 according the protocol described in Scheme VIII.

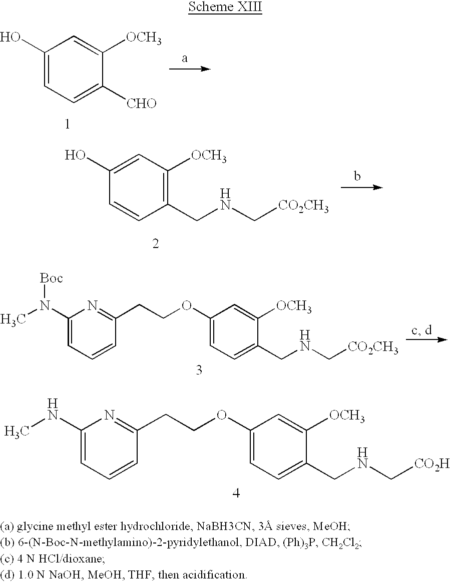

A suitably functionalized aromatic aldehyde such as 4-hydroxy-2-methoxybenzaldehyde (XIII-1), is reacted with an amino acid derivative, for instance glycine methyl ester hydrochloride, under reductive amination conditions, to afford XIII-2. Reductive amination involves the reaction of an aldehyde or ketone with an amine in the presence of a suitable reducing agent, generally sodium cyanoborohydride (NaBH₃CN) or sodium triacetoxyborohydride (NaB(OAc)₃H), oftentimes in the presence of an acid catalyst, generally acetic acid or hydrochloric acid. The reaction proceeds through an intermediate imine, which reacts in situ with the reducing agent to afford the amine. Alternatively, the imine can be prepared as a discreet entity, and reduced in a subsequent step. Typical solvents for this reaction include CH₂Cl₂, DMF, or an alcohol such as MeOH or EtOH. A dehydrating reagent, such as molecular sieves, MgSO₄, or trimethyl orthoformate, can be used to react with the water liberated during the course of the reaction. The product XIII-2 is subsequently converted to XIII-4 according the protocol described in Scheme VIII.

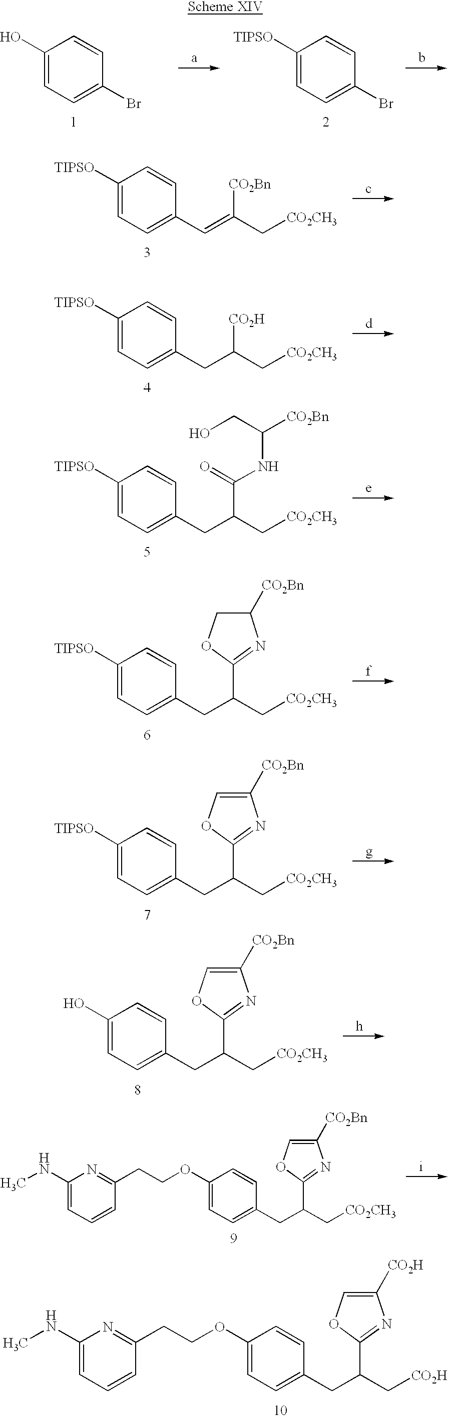

A halophenol derivative, for instance 4-bromophenol (XIV-1), is converted to a suitably protected derivative, for instance 4-bromo-1-(triisopropylsilyloxy)benzene (XIV-2). The protecting group for the phenol must be compatible with subsequent chemistry, and also must be able to be removed selectively when desired. Methods for the protection of phenols are described in standard reference volumes, such as Greene, “Protective Groups in Organic Synthesis” (published by Wiley-Interscience). XIV-2 is converted to XIV-4 and subsequently to XIV-5 according to the general methods described in Scheme X. XIV-5 is then converted to the oxazole derivative XIV-7. Several methods are known for the conversion of amidoalcohols to oxazoles (Meyers, Tetrahedron 1994, 50, 2297-2360; Wipf, J. Org. Chem. 1993, 58, 3604-3606). For example, the amidoalcohol XIV-5 can be converted first to the oxazoline XIV-6. This transformation is generally accomplished under dehydrating conditions, such as reaction with Burgess reagent in THF. Oxazoline XIV-6 is then oxidized to oxazole XIV-7 using, for instance, bromtrichloromethane and DBU in CH₂Cl₂ (Williams, Tetrahedron Letters 1997, 38, 331-334) or CuBr₂ and DBU in an appropriate solvent, such as EtOAc/CHCl₃ or CH₂Cl₂ (Barrish, J. Org. Chem. 1993, 58, 4494-4496). Removal of the silyl protecting group affords phenol XIV-8, which is converted to XIV-10 as described in Scheme V.

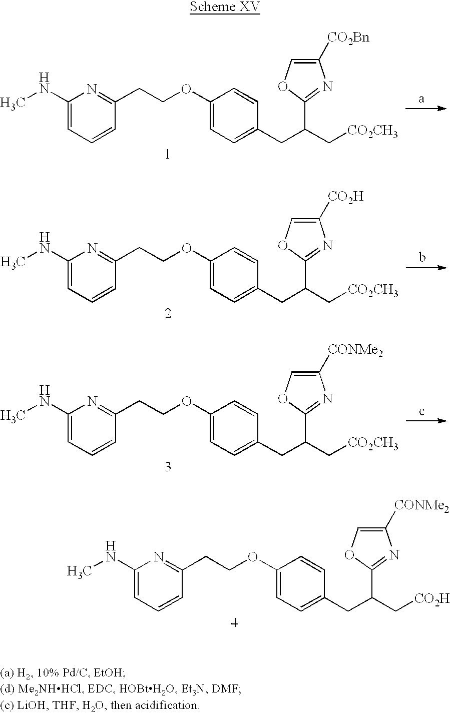

Compound XV-1, prepared as described in Scheme XIV, is converted to the carboxylic acid derivative XV-2 by hydrogenation in the presence of a suitable catalyst, preferably palladium metal on activated carbon (Pd/C), in an inert solvent, generally MeOH, EtOH, EtOAc, or mixtures thereof. XV-2 is converted to the amide derivative XV-3 according to the general methods for formation of amides from carboxylic acids described in Scheme X. Saponification as described in Scheme V gives XV-4.

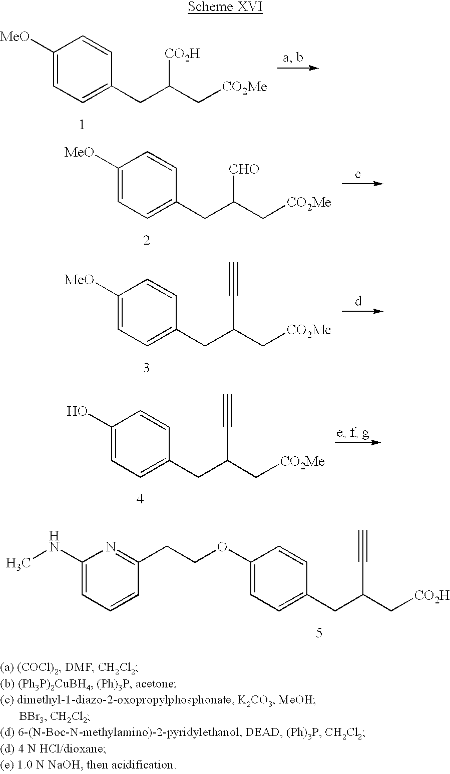

Compound XVI-1, prepared as described in Scheme X, is converted to aldehyde derivative XVI-2, preferably by the method of Fleet and Harding (Tet. Lett. 1979, 11, 975-978). This method involves initial conversion of the carboxylic acid moiety of XVI-1 to the corresponding acid chloride under standard conditions well-known to those of skill in the art, followed by reduction to the aldehyde using (Ph₃P)₂CuBH₄. Other methods are known for the selective conversion of a carboxylic acid to an aldehyde in the presence of a carboxylic ester, and can be found in standard reference volumes, such as Compendium of Organic Synthetic Methods (published by Wiley-Interscience). The aldehyde XVI-2 is subsequently transformed into the acetylene derivative XVI-3 by the procedure of Muller, et al. (Syn. Lett. 1996, 521-522). Thus, XVI-2 is reacted with dimethyl-1-diazo-2-oxopropylphosphonate in the presence of a suitable base, generally K₂CO₃, in an appropriate solvent, such as methanol. Additional methods for the conversion of an aldehyde to an acetylene are known, and can be found in standard reference volumes, such as Compendium of Organic Synthetic Methods (published by Wiley-Interscience). The product XVI-3 is subsequently converted to XVI-5 according the general protocol described in Scheme VIII.

Amide coupling reagents as used herein denote reagents which may be used to form peptide bonds. Typical coupling methods employ carbodiimides, activated anhydrides and esters and acyl halides. Reagents such as EDC, DCC, DPPA, BOP reagent, HOBt, N-hydroxysuccinimide and oxalyl chloride are typical.

Coupling methods to form peptide bonds are generally well known to the art. The methods of peptide synthesis generally set forth by Bodansky el al., THE PRACTICE OF PEPTIDE SYNTHESIS, Springer-Verlag, Berlin, 1984, Ali et al. in J. Med. Chem., 29, 984 (1986) and J. Med. Chem., 30, 2291 (1987) are generally illustrative of the technique and are incorporated herein by reference.

Typically, the amine or aniline is coupled via its free amino group to an appropriate carboxylic acid substrate using a suitable carbodiimide coupling agent, such as N,N′dicyclohexyl carbodiimide (DCC), optionally in the presence of catalysts such as 1-hydroxybenzotriazole (HOBt) and dimethylamino pyridine (DMAP). Other methods, such as the formation of activated esters, anhydrides or acid halides, of the free carboxyl of a suitably protected acid substrate, and subsequent reaction with the free amine of a suitably protected amine, optionally in the presence of a base, are also suitable. For example, a protected Boc-amino acid or Cbz-amidino benzoic acid is treated in an anhydrous solvent, such as methylene chloride or tetrahydrofuran(THF), in the presence of a base, such as N-methyl morpholine, DMAP or a trialkylamine, with isobutyl chloroformate to form the “activated anhydride”, which is subsequently reacted with the free amine of a second protected amino acid or aniline.

Useful intermediates for preparing formula (I) compounds in which R² is a benzimidazole are disclosed in Nestor et al, J. Med. Chem. 1984, 27, 320. Representative methods for preparing benzimidazole compounds useful as intermediates in the present invention are also common to the art and may be found, for instance, in EP-A 0 381 033.

Acid addition salts of the compounds are prepared in a standard manner in a suitable-solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts.

This invention also provides a pharmaceutical composition which comprises a compound according to formula (I) and a pharmaceutically acceptable carrier. Accordingly, the compounds of formula (I) may be used in the manufacture of a medicament. Pharmaceutical compositions of the compounds of formula (I) prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, these compounds may be encapsulated, tableted or prepared in a emulsion or syrup for oral administration. Pharmaceutically acceptable solid or liquid carriers may be added to enhance or stabilize the composition, or to facilitate preparation of the composition. Solid carriers include starch, lactose. calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

For rectal administration, the compounds of this invention may also be combined with excipients such as cocoa butter, glycerin, gelatin or polyethylene glycols and molded into a suppository.

The compounds described herein are antagonists of the vitronectin receptor, and are useful for treating diseases wherein the underlying pathology is attributable to ligand or cell which interacts with the vitronectin receptor. For instance, these compounds are useful for the treatment of diseases wherein loss of the bone matrix creates pathology. Thus. the instant compounds are useful for the treatment of ostoeporosis, hyperparathyroidism. Paget's disease, hypercalcemia of malignancy, osteolytic lesions produced by bone metastasis, bone loss due to immobilization or sex hormone deficiency. The compounds of this invention are also believed to have utility as antitumor, anti-angiogenic, antiinflammatory and anti-metastatic agents, and be useful in the treatment of atherosclerosis and restenosis.

The compound is administered either orally or parenterally to the patient, in a manner such that the concentration of drug is sufficient to inhibit bone resorption, or other such indication. The pharmaceutical composition containing the compound is administered at an oral dose of between about 0.1 to about 50 mg/kg in a manner consistent with the condition of the patient. Preferably the oral dose would be about 0.5 to about 20 mg/kg. For acute therapy, parenteral administration is preferred. An intravenous infusion of the peptide in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg. The compounds are administered one to four times daily at a level to achieve a total daily dose of about 0.4 to about 400 mg/kg/day. The precise level and method by which the compounds are administered is readily determined by one routinely skilled in the art by comparing the blood level of the agent to the concentration required to have a therapeutic effect.

This invention further provides a method for treating osteoporosis or inhibiting bone loss which comprises administering stepwise or in physical combination a compound of formula (I) and other inhibitors of bone resorption, such as bisphosphonates (i.e., allendronate), hormone replacement therapy, anti-estrogens, or calcitonin. In addition, this invention provides a method of treatment using a compound of this invention and an anabolic agent, such as the bone morphogenic protein, iproflavone, useful in the prevention of bone loss and/or to increase bone mass.

Additionally, this invention provides a method of inhibiting tumor growth which comprises administering stepwise or in physical combination a compound of formula (I) and an antineoplastic agent. Compounds of the camptothecin analog class, such as topotecan, irinotecan and 9-aminocamptothecin, and platinum coordination complexes, such as cisplatin, ormaplatin and tetraplatin, are well known groups of antineoplastic agents. Compounds of the camptothecin analog class are described in U.S. Pat. Nos. 5,004,758, 4,604,463, 4,473,692, 4,545,880 4,342,776, 4,513,138, 4,399,276, EP Patent Application Publication Nos. 0 418 099 and 0 088 642, Wani, et al., J. Med. Chem., 1986, 29, 2358, Wani, et al., J. Med. Chem., 1980, 23, 554, Wani, et al., J. Med. Chem., 1987, 30, 1774, and Nitta, et al., Proc. 14th International Congr. Chemotherapy., 1985, Anticancer Section 1, 28, the entire disclosure of each which is hereby incorporated by reference. The platinum coordination complex, cisplatin, is available under the name Platinol® from Bristol Myers-Squibb Corporation. Useful formulations for cisplatin are described in U.S Pat. Nos. 5,562,925 and 4,310,515, the entire disclosure of each which is hereby incorporated by reference.

In the method of inhibiting tumor growth which comprises administering stepwise or in physical combination a compound of formula (I) and an antineoplastic agent, the platinum coordination compound, for example cisplatin, can be administered using slow intravenous infusion. The preferred carrier is a dextrose/saline solution containing mannitol. The dose schedule of the platinum coordination compound may be on the basis of from about 1 to about 500 mg per square meter (mg/m²) of body surface area per course of treatment. Infusions of the platinum coordiation compound may be given one to two times weekly, and the weekly treatments may be repeated several times. Using a compound of the camptothecin analog class in a parenteral administration, the course of therapy generally employed is from about 0.1 to about 300.0 mg/m² of body surface area per day for about five consecutive days. Most preferably, the course of therapy employed for topotecan is from about 1.0 to about 2.0 mg/m² of body surface area per day for about five consecutive days. Preferably, the course of therapy is repeated at least once at about a seven day to about a twenty-eight day interval.

The pharmaceutical composition may be formulated with both the compound of formula (I) and the antineoplastic agent in the same container, but formualtion in different containers is preferred. When both agents are provided in solution form, they can be contained in an infusion/injection system for simultaneous administration or in a tandem arrangement.

For convenient administration of the compound of formula (I) and the antineoplastic agent at the same or different times, a kit is prepared, comprising, in a single container, such as a box, carton or other container, individual bottles, bags, vials or other containers each having an effective amount of the compound of formula (I) for parenteral administration, as described above, and an effective amount of the antineoplastic agent for parenteral administration, as described above. Such kit can comprise, for example, both pharmaceutical agents in separate containers or the same container, optionally as lyophilized plugs, and containers of solutions for reconstitution. A variation of this is to include the solution for reconstitution and the lyophilized plug in two chambers of a single container, which can be caused to admix prior to use. With such an arrangement, the antineoplastic agent and the compound of this invention may be packaged separately, as in two containers, or lyophilized together as a powder and provided in a single container.

When both agents are provided in solution form, they can be contained in an infusion/injection system for simultaneous administration or in a tandem arrangement. For example, the compound of formula (I) may be in an i.v. injectable form, or infusion bag linked in series, via tubing, to the antineoplastic agent in a second infusion bag. Using such a system, a patient can receive an initial bolus-type injection or infusion of the compound of formula (I) followed by an infusion of the antineoplastic agent.

The compounds may be tested in one of several biological assays to determine the concentration of compound which is required to have a given pharmacological effect.

Inhibition of Vitronectin Binding

Solid-Phase [³H]-SK&F-107260 Binding to αᵥβ₃: Human placenta or human platelet αᵥβ₃ (0.1-0.3 mg/mL) in buffer T (containing 2 mM CaCl₂ and 1% octylglucoside) was diluted with buffer T containing 1 mM CaCl₂, 1 mM MnCl₂, 1 mM MgCl₂ (buffer A) and 0.05% NaN₃, and then immediately added to 96-well ELISA plates (Corning, New York, N.Y.) at 0.1 mL per well. 0.1-0.2 μg of αᵥβ₃ was added per well. The plates were incubated overnight at 4° C. At the time of the experiment, the wells were washed once with buffer A and were incubated with 0.1 mL of 3.5% bovine serum albumin in the same buffer for 1 hr at room temperature. Following incubation the wells were aspirated completely and washed twice with 0.2 mL buffer A.

Compounds were dissolved in 100% DMSO to give a 2 mM stock solution, which was diluted with binding buffer (15 mM Tris-HCl (pH 7.4), 100 mM NaCl, 1 mM CaCl₂, 1 mM MnCl₂, 1 mM MgCl₂) to a final compound concentration of 100 μM. This solution is then diluted to the required final compound concentration. Various concentrations of unlabeled antagonists (0.001-100 μM) were added to the wells in triplicates, followed by the addition of 5.0 nM of [³H]-SK&F-107260 (65-86 Ci/mmol).

The plates were incubated for 1 hr at room temperature. Following incubation the wells were aspirated completely and washed once with 0.2 mL of ice cold buffer A in a well-to-well fashion. The receptors were solubilized with 0.1 mL of 1% SDS and the bound [³H]-SK&F-107260 was determined by liquid scintillation counting with the addition of 3 mL Ready Safe in a Beckman LS Liquid Scintillation Counter, with 40% efficiency. Nonspecific binding of [³H]-SK&F-107260 was determined in the presence of 2 μM SK&F-107260 and was consistently less than 1% of total radioligand input. The IC₅₀ (concentration of the antagonist to inhibit 50% binding of [³H]-SK&F-107260) was determined by a nonlinear, least squares curve-fitting routine, which was modified from the LUNDON-2 program. The Kᵢ (dissociation constant of the antagonist) was calculated according to the equation: Kᵢ=IC₅₀/(1+L/K_{d}), where L and K_{d} were the concentration and the dissociation constant of [³H]-SK&F-107260, respectively.

Compounds of the present invention inhibit vitronectin binding to SK&F 107260 in the concentration range of about 10 to about 0.01 micomolar.

Compounds of this invention are also tested for in vitro and in vivo bone resorption in assays standard in the art for evaluating inhibition of bone formation, such as the pit formation assay disclosed in EP 528 587, which may also be performed using human osteoclasts in place of rat osteoclasts, and the ovarectomized rat model, described by Wronski et al., Cells and Materials 1991, Sup. 1, 69-74.

Vascular Smooth Muscle Cell Migration Assay

Rat or human aortic smooth muscle cells were used. The cell migration was monitored in a Transwell cell culture chamber by using a polycarbonate membrane with pores of 8 um (Costar). The lower surface of the filter was coated with vitronectin. Cells were suspended in DMEM supplemented with 0.2% bovine serum albumin at a concentration of 2.5-5.0×10⁶ cells/mL, and were pretreated with test compound at various concentrations for 20 min at 20° C. The solvent alone was used as control. 0.2 mL of the cell suspension was placed in the upper compartment of the chamber. The lower compartment contained 0.6 mL of DMEM supplemented with 0.2% bovine serum albumin. Incubation was carried out at 37° C. in an atmosphere of 95% air/5% CO₂ for 24 hr. After incubation, the non-migrated cells on the upper surface of the filter were removed by gentle scraping. The filter was then fixed in methanol and stained with 10% Giemsa stain. Migration was measured either by a) counting the number of cells that had migrated to the lower surface of the filter or by b) extracting the stained cells with 10% acetic acid followed by determining the absorbance at 600 nM.

Thyroparathyroidectomized Rat Model

Each experimental group consists of 5-6 adult male Sprague-Dawley rats (250-400g body weight). The rats are thyroparathyroidectomized (by the vendor, Taconic Farms) 7 days prior to use. All rats receive a replacement dose of thyroxine every 3 days. On receipt of the rats, circulating ionized calcium levels are measured in whole blood immediately after it has been withdrawn by tail venipuncture into heparinized tubes. Rats are included if the ionized Ca level (measured with a Ciba-Corning model 634 calcium pH analyzer) is <1.2 mM/L. Each rat is fitted with an indwelling venous and arterial catheter for the delivery of test material and for blood sampling respectively. The rats are then put on a diet of calcium-free chow and deionized water. Baseline Ca levels are measured and each rat is administered either control vehicle or human parathyroid hormone 1-34 peptide (hPTH1-34, dose 1.25 ug/kg/h in saline/0.1% bovine serum albumin, Bachem, Ca) or a mixture of hPTH 1-34 and test material, by continuous intravenous infusion via the venous catheter using an external syringe pump. The calcemic response of each rat is measured at two-hourly intervals during the infusion period of 6-8 hours.

Human Osteoclast Resorption and Adhesion Assays

Pit resorption and adhesion assays have been developed and standardized using normal human osteoclasts derived from osteoclastoma tissue. Assay 1 was developed for the measurement of osteoclast pit volumes by laser confocal microscopy. Assay 2 was developed as a higher throughput screen in which collagen fragments (released during resorption) are measured by competitive ELISA.

Assay 1 (Using Laser Confocal Microscopy)

Aliquots of human osteoclastoma-derived cell suspensions are removed from liquid nitrogen strorage, warmed rapidly at 37° C. and washed ×1 in RPMI-1640 medium by centrifugation (1000 rpm, 5 mins at 4° C.).

The medium is aspirated and replaced with murine anti-HLA-DR antibody then diluted 1:3 in RPMI-1640 medium. The suspension is incubated for 30 mins on ice and mixed frequently.

The cells are washed ×2 with cold RPMI-1640 followed by centrifugation (1000 rpm, 5 mins at 4° C.) and the cells are then transferred to a sterile 15 ml centrifuge tube. The number of mononuclear cells are enumerated in an improved Neubauer counting chamber.

Sufficient magnetic beads (5/mononuclear cell), coated with goat anti-mouse IgG (Dynal, Great Neck, N.Y.) are removed from their stock bottle and placed into 5 ml of fresh medium (this washes away the toxic azide preservative). The medium is removed by immobilizing the beads on a magnet and is replaced with fresh medium.

The beads are mixed with the cells and the suspension is incubated for 30 mins on ice. The suspension is mixed frequently.

The bead-coated cells are immobilized on a magnet and the remaining cells (osteoclast-rich fraction) are decanted into a sterile 50 ml centrifuge tube.

Fresh medium is added to the bead-coated cells to dislodge any trapped osteoclasts. This wash process is repeated ×10. The bead-coated cells are discarded.

The viable osteoclasts are enumerated in a counting chamber, using fluorescein diacetate to label live cells. A large-bore disposable plastic pasteur pipet is used to add the sample to the chamber.

The osteoclasts are pelleted by centrifugation and the density adjusted to the appropriate number in EMEM medium (the number of osteoclasts is variable from tumor to tumor), supplemented with 10% fetal calf serum and 1.7g/liter of sodium bicarbonate.

3 ml aliquots of the cell suspension (per compound treatment) are decanted into 15 ml centrifuge tubes. The cells are pelleted by centrifugation.

To each tube, 3 ml of the appropriate compound treatment are added (diluted to 50 uM in the EMEM medium). Also included are appropriate vehicle controls, a positive control (anti-vitronectin receptor murine monoclonal antibody [87MEM1] diluted to 100 ug/ml) and an isotype control (IgG₂ₐ diluted to 100 ug/ml). The samples are incubated at 37° C. for 30 mins.

0.5 ml aliquots of the cells are seeded onto sterile dentine slices in a 48-well plate and incubated at 37° C. for 2 hours. Each treatment is screened in quadruplicate.

The slices are washed in six changes of warm PBS (10 ml/well in a 6-well plate) and then placed into fresh medium containing the compound treatment or control samples. The samples are incubated at 37° C. for 48 hours.

Tartrate Resistant Acid Phosphatase (TRAP) Procedure (Selective Stain for Cells of the Osteoclast Lineage)

The bone slices containing the attached osteoclasts are washed in phosphate buffered saline and fixed in 2% gluteraldehyde (in 0.2M sodium cacodylate) for 5 mins.

They are then washed in water and are incubated for 4 minutes in TRAP buffer at 37° C. (0.5 mg/ml naphthol AS-BI phosphate dissolved in N,N-dimethylformamide and mixed with 0.25 M citrate buffer (pH 4.5), containing 100 mM sodium tartrate.

Following a wash in cold water the slices are immersed in cold acetate buffer (0.1 M, pH 6.2) containing 1 mg/ml fast red garnet and incubated at 4° C. for 4 minutes.

Excess buffer is aspirated, and the slices are air dried following a wash in water.

The TRAP positive osteoclasts (brick red/purple precipitate) are enumerated by bright-field microscopy and are then removed from the surface of the dentine by sonication.

Pit volumes are determined using the Nikon/Lasertec ILM21W confocal microscope.

Assay 2 (Using an ELISA Readout)

The human osteoclasts are enriched and prepared for compound screening as described in the initial 9 steps of Assay 1. For clarity, these steps are repeated hereinbelow.

Aliquots of human osteoclastoma-derived cell suspensions are removed from liquid nitrogen strorage, warmed rapidly at 37° C. and washed ×1 in RPMI-1640 medium by centrifugation (1000 rpm, 5 mins at 4° C.).

The medium is aspirated and replaced with murine anti-HLA-DR antibody then diluted 1.3 in RPMI-1640 medium. The suspension is incubated for 30 mins on ice and mixed frequently.

The cells are washed ×2 with cold RPMI-1640 followed by centrifugation (1000 rpm, 5 mins at 4° C.) and the cells are then transferred to a sterile 15 ml centrifuge tube. The number of mononuclear cells are enumerated in an improved Neubauer counting chamber.

Sufficient magnetic beads (5/mononuclear cell), coated with goat anti-mouse IgG (Dynal, Great Neck, N.Y.) are removed from their stock bottle and placed into 5 ml of fresh medium (this washes away the toxic azide preservative). The medium is removed by immobilizing the beads on a magnet and is replaced with fresh medium.

The beads are mixed with the cells and the suspension is incubated for 30 mins on ice. The suspension is mixed frequently.

The bead-coated cells are immobilized on a magnet and the remaining cells (osteoclast-rich fraction) are decanted into a sterile 50 ml centrifuge tube.

Fresh medium is added to the bead-coated cells to dislodge any trapped osteoclasts. This wash process is repeated ×10. The bead-coated cells are discarded.

The viable osteoclasts are enumerated in a counting chamber, using fluorescein diacetate to label live cells. A large-bore disposable plastic pasteur pipet is used to add the sample to the chamber.

The osteoclasts are pelleted by centrifugation and the density adjusted to the appropriate number in EMEM medium (the number of osteoclasts is variable from tumor to tumor), supplemented with 10% fetal calf serum and 1.7 g/liter of sodium bicarbonate.

In contrast to the method desribed above in Assay 1, the compounds are screened at 4 doses to obtain an IC₅₀, as outlined below:

The osteoclast preparations are preincubated for 30 minutes at 37° C. with test compound (4 doses) or controls.

They are then seeded onto bovine cortical bone slices in wells of a 48-well tissue culture plate and are incubated for a further 2 hours at 37° C.

The bone slices are washed in six changes of warm phosphate buffered saline (PBS), to remove non-adherent cells, and are then returned to wells of a 48 well plate containing fresh compound or controls.

The tissue culture plate is then incubated for 48 hours at 37° C.

The supernatants from each well are aspirated into individual tubes and are screened in a competitive ELISA that detects the c-telopeptide of type I collagen which is released during the resorption process. This is a commercially available ELISA (Osteometer, Denmark) that contains a rabbit antibody that specifically reacts with an 8-amino acid sequence (Glu-Lys-Ala-His-Asp-Gly-Gly-Arg) that is present in the carboxy-terminal telopeptide of the a1-chain of type I collagen. The results are expressed as % inhibition of resorption compared to a vehicle control.

Human Osteoclast Adhesion Assay

The human osteoclasts are enriched and prepared for compound screening as described above in the inital 9 steps of Assay 1. For clarity, these steps are repeated hereinbelow.

Aliquots of human osteoclastoma-derived cell suspensions are removed from liquid nitrogen strorage, warmed rapidly at 37° C. and washed ×1 in RPMI-1640 medium by centrifugation (1000 rpm, 5 mins at 4° C.).

The medium is aspirated and replaced with murine anti-HLA-DR antibody then diluted 1:3 in RPMI-1640 medium. The suspension is incubated for 30 mins on ice and mixed frequently.

The cells are washed ×2 with cold RPMI-1640 followed by centrifugation (1000 rpm, 5 mins at 4° C.) and the cells are then transferred to a sterile 15 ml centrifuge tube. The number of mononuclear cells are enumerated in an improved Neubauer counting chamber.

Sufficient magnetic beads (5/mononuclear cell), coated with goat anti-mouse IgG (Dynal, Great Neck, N.Y.) are removed from their stock bottle and placed into 5 ml of fresh medium (this washes away the toxic azide preservative). The medium is removed by immobilizing the beads on a magnet and is replaced with fresh medium.

The beads are mixed with the cells and the suspension is incubated for 30 mins on ice. The suspension is mixed frequently.

The bead-coated cells are immobilized on a magnet and the remaining cells (osteoclast-rich fraction) are decanted into a sterile 50 ml centrifuge tube.

Fresh medium is added to the bead-coated cells to dislodge any trapped osteoclasts. This wash process is repeated ×10. The bead-coated cells are discarded.

The viable osteoclasts are enumerated in a counting chamber, using fluorescein diacetate to label live cells. A large-bore disposable plastic pasteur pipet is used to add the sample to the chamber.

The osteoclasts are pelleted by centrifugation and the density adjusted to the appropriate number in EMEM medium (the number of osteoclasts is variable from tumor to tumor), supplemented with 10% fetal calf serum and 1.7 g/liter of sodium bicarbonate.

Osteoclastoma-derived osteoclasts are preincubated with compound (4 doses) or controls at 37° C. for 30 minutes.

The cells are then seeded onto osteopontin-coated slides (human or rat osteopontin, 2.5 ug/ml) and incubated for 2 hours at 37° C.

Non adherent cells are removed by washing the slides vigorously in phosphate buffered saline and the cells remaining on the slides are fixed in acetone.

The osteoclasts are stained for tartrate-resistant acid phosphatase (TRAP), a selective marker for cells of this phenotype (see steps 15-17), and are enumerated by light microscopy. The results are expressed as % inhibition of adhesion compared to a vehicle control.

Cell Adhesion Assay

Cells and Cell Culture

Human embryonic kidney cells (HEK293 cells) were obtained from ATCC (Catalog No. CRL 1573). Cells were grown in Earl's minimal essential medium (EMEM) medium containing Earl's salts, 10% fetal bovine serum, 1% glutamine and 1% Penicillin-Steptomycin.

Constructs and Transfections

A 3.2 kb EcoRI-KpnI fragment of the αᵥ subunit and a 2.4 kb XbaI-XhoI fragment of the β₃ subunit were inserted into the EcoRI-EcoRV cloning sites of the pCDN vector (Aiyar et al., 1994) which contains a CMV promoter and a G418 selectable marker by blunt end ligation. For stable expression, 80×10⁶ HEK 293 cells were electrotransformed with αᵥ+β₃ constructs (20 μg DNA of each subunit) using a Gene Pulser (Hensley et al., 1994) and plated in 100 mm plates (5×10⁵ cells/plate). After 48 hr, the growth medium was supplemented with 450 μg/mL Geneticin (G418 Sulfate, GIBCO-BRL, Bethesda, Md.). The cells were maintained in selection medium until the colonies were large enough to be assayed.

Immunocytochemical Analysis of Transfected Cells

To determine whether the HEK 293 transfectants expressed the vitronectin receptor, the cells were immobilized on glass microscope slides by centrifugation, fixed in acetone for 2 min at room temperature and air dried. Specific reactivity with 23C6, a monoclonal antibody specific for the α_{vβ}₃ complex was demonstrated using a standard indirect immunofluorescence method.

Cell Adhesion Studies

Corning 96-well ELISA plates were precoated overnight at 4° C. with 0.1 ml of human vitronectin (0.2 μg/mL in RPMI medium). At the time of the experiment, the plates were washed once with RPMI medium and blocked with 3.5% BSA in RPMI medium for 1 hr at room temperature. Transfected 293 cells were resuspended in RPMI medium, supplemented with 20 mM Hepes, pH 7.4 and 0.1% BSA at a density of 0.5×10⁶ cells/mL. 0.1 mL of cell suspension was added to each well and incubated for 1 hr at 37° C., in the presence or absence of various α_{vβ}₃ antagonists. Following incubation, 0.025 mL of a 10% formaldehyde solution, pH 7.4, was added and the cells were fixed at room temperature for 10 min. The plates were washed 3 times with 0.2 mL of RPMI medium and the adherent cells were stained with 0.1 mL of 0.5% toluidine blue for 20 min at room temperature. Excess stain was removed by extensive washing with deionized water. The toluidine blue incorporated into cells was eluted by the addition of 0.1 mL of 50% ethanol containing 50 mM HCl. Cell adhesion was quantitated at an optical density of 600 nm on a microtiter plate reader (Titertek Multiskan MC, Sterling, Va.).

Solid-Phase α_{vβ}₅ Binding Assay:

The vitronectin receptor αᵥβ₅ was purified from human placenta. Receptor preparation was diluted with 50 mM Tris-HCl, pH 7.5, 100 mM NaCl, 1 mM CaCl₂, 1 mM MnCl₂, 1 mM MgCl₂ (buffer A) and was immediately added to 96-well ELISA plates at 0.1 ml per well. 0.1-0.2 μg of αᵥβ₃ was added per well. The plates were incubated overnight at 4° C. At the time of the experiment, the wells were washed once with buffer A and were incubated with 0.1 ml of 3.5% bovine serum albumin in the same buffer for 1 hr at room temperature. Following incubation the wells were aspirated completely and washed twice with 0.2 ml buffer A.

In a [³H]-SK&F-107260 competition assay, various concentrations of unlabeled antagonists (0.001-100 μM) were added to the wells, followed by the addition of 5.0 nM of [³H]-SK&F-107260. The plates were incubated for 1 hr at room temperature. Following incubation the wells were aspirated completely and washed once with 0.2 ml of ice cold buffer A in a well-to-well fashion. The receptors were solubilized with 0.1 ml of 1% SDS and the bound [³H]-SK&F-107260 was determined by liquid scintillation counting with the addition of 3 ml Ready Safe in a Beckman LS 6800 Liquid Scintillation Counter, with 40% efficiency. Nonspecific binding of [³H]-SK&F-107260 was determined in the presence of 2 μM SK&F-107260 and was consistently less than 1% of total radioligand input. The IC₅₀ (concentration of the antagonist to inhibit 50% binding of [³H]-SK&F-107260) was determined by a nonlinear, least squares curve-fitting routine, which was modified from the LUNDON-2 program. The Kᵢ (dissociation constant of the antagonist) was calculated according to Cheng and Prusoff equation: Kᵢ=IC₅₀/(1+L/K_{d}), where L and K_{d} were the concentration and the dissociation constant of [3H]-SK&F-107260, respectively.

### Inhibition of RGD-mediated GPIIb-IIIa Binding

Purification of GPIIb-IIIa

Ten units of outdated, washed human platelets (obtained from Red Cross) were lyzed by gentle stirring in 3% octylglucoside, 20 mM Tris-HCl, pH 7.4, 140 mM NaCl, 2 mM CaCl₂ at 40° C. for 2 h. The lysate was centrifuged at 100,000 g for 1 h. The supernatant obtained was applied to a 5 mL lentil lectin sepharose 4B column (E.Y. Labs) preequilibrated with 20 mM Tris-HCl, pH 7.4, 100 mM NaCl, 2 mM CaCl₂, 1% octylglucoside (buffer A). After 2 h incubation, the column was washed with 50 mL cold buffer A. The lectin-retained GPIIb-IIIa was eluted with buffer A containing 10% dextrose. All procedures were performed at 4° C. The GPlIb-IIIa obtained was >95% pure as shown by SDS polyacrylamide gel electrophoresis.

Incorporation of GPIIb-IIIa in Liposomes.

A mixture of phosphatidylserine (70%) and phosphatidylcholine (30%) (Avanti Polar Lipids) were dried to the walls of a glass tube under a stream of nitrogen. Purified GPIlb-IIIa was diluted to a final concentration of 0.5 mg/mL and mixed with the phospholipids in a protein:phospholipid ratio of 1:3 (w:w). The mixture was resuspended and sonicated in a bath sonicator for 5 min. The mixture was then dialyzed overnight using 12,000-14,000 molecular weight cutoff dialysis tubing against a 1000-fold excess of 50 mM Tris-HCl, pH 7.4, 100 mM NaCl, 2 mM CaCl2 (with 2 changes). The GPIIb-IIIa-containing liposomes wee centrifuged at 12,000 g for 15 min and resuspended in the dialysis buffer at a final protein concentration of approximately 1 mg/mL. The liposomes were stored at −70° C. until needed.

Competitive Binding to GPIlb-IIIa

The binding to the fibrinogen receptor (GPIlb-IIIa) was assayed by an indirect competitive binding method using [³H]-SK&F-107260 as an RGD-type ligand. The binding assay was performed in a 96-well filtration plate assembly (Millipore Corporation, Bedford, Mass.) using 0.22 um hydrophilic durapore membranes. The wells were precoated with 0.2 mL of 10 μg/mL polylysine (Sigma Chemical Co., St. Louis, Mo.) at room temperature for 1 h to block nonspecific binding. Various concentrations of unlabeled benzazepines were added to the wells in quadruplicate. [³H]-SK&F-107260 was applied to each well at a final concentration of 4.5 nM, followed by the addition of 1 μg of the purified platelet GPIIb-IIIa-containing liposomes. The mixtures were incubated for 1 h at room temperature. The GPIIb-IIIa-bound [3H]-SK&F-107260 was seperated from the unbound by filtration using a Millipore filtration manifold, followed by washing with ice-cold buffer (2 times, each 0.2 mL). Bound radioactivity remaining on the filters was counted in 1.5 mL Ready Solve (Beckman Instruments, Fullerton, Calif.) in a Beckman Liquid Scintillation Counter (Model LS6800), with 40% efficiency. Nonspecific binding was determined in the presence of 2 μM unlabeled SK&F-107260 and was consistently less than 0.14% of the total radioactivity added to the samples. All data points are the mean of quadruplicate determinations.

Competition binding data were analyzed by a nonlinear least-squares curve fitting procedure. This method provides the IC50 of the antagonists (concentration of the antagonist which inhibits specific binding of [³H]-SK&F-107260 by 50% at equilibrium). The IC50 is related to the equilibrium dissociation constant (Ki) of the antagonist based on the Cheng and Prusoff equation: Ki=IC50/(1+L/Kd), where L is the concentration of [3H]-SK&F-107260 used in the competitive binding assay (4.5 nM), and Kd is the dissociation constant of [3H]-SK&F-107260 which is 4.5 nM as determined by Scatchard analysis.

Preferred compounds of this invention have an affinity for the vitronectin receptor relative to the fibrinogen receptor of greater than 10:1. Most preferred compounds have a ratio of activity of greater than 100:1.

The efficacy of the compounds of formula (I) alone or in combination with an antineoplastic agent may be determined using several transplantable mouse tumor models. See U.S. Pat. Nos. 5,004,758 and 5,633,016 for details of these models

The examples which follow are intended in no way to limit the scope of this invention, but are provided to illustrate how to make and use the compounds of this invention. Many other embodiments will be readily apparent to those skilled in the art.

### General

Proton nuclear magnetic resonance (¹H NMR) spectra were recorded at either 250, 300, or 400 MHz. Chemical shifts are reported in parts per million (δ) downfield from the internal standard tetramethylsilane (TMS). Abbreviations for NMR data are as follows: s=srnglet, d=doublet, t=triplet, q=quartet, m=multiplet, dd=doublet of doublets, dt=doublet of triplets, app=apparent, br=broad. J indicates the NMR coupling constant measured in Hertz. CDCl₃ is deuteriochloroform, DMSO-d₆ is hexadeuteriodimethylsulfoxide, and CD₃OD is tetradeuteriomethanol. Infrared (IR) spectra were recorded in transmission mode, and band positions are reported in inverse wavenumbers (cm⁻¹). Mass spectra were obtained using electrospray (ES) or FAB ionization techniques. Elemental analyses were performed either in-house or by Quantitative Technologies Inc., Whitehouse, N.J. Melting 50° C., and the resulting residue was allowed to rotate on the rotavap at 50° C. under vacuum. After 21.5 hr, the reaction was diluted with hexanes (400 mL) and filtered through silica gel (hexanes followed by 20% EtOAc/hexanes). Concentration left the title compound (41.84 g, quantitative) as a light yellow oil which gradually solidified on standing: ¹H NMR (250 MHz, CDCl₃) δ7.71 (d, J=8.3 Hz, 1 H), 7.40-7.65 (m, 2 H), 6.80 (d, J=7.5 Hz, 1 H), 2.43 (s, 3 H), 1.50 (s, 9 H); MS (ES) m/e 153 (M+H−C₄H₈)⁺.

### b) 2-[(tert-Butoxycarbonyl)methylamino]-6-picoline

NaH (60% in mineral oil, 3.60 g, 90 mmole) was added in portions over several min to a solution of 2-(tert-butoxycarbonylamino)-6-picoline (15.62 g, 75 mmole) and iodomethane (9.3 mL, 150 mmole) in anhydrous DMSO (75 mL) at 15° C. (cool water bath). The internal temperature rose to 35° C. When gas evolution had subsided, the cool water bath was removed and the reaction was allowed to stir at RT. After 0.5 hr, the dark yellow mixture was poured onto ice/H₂O (300 mL) and extracted with Et₂O (3×300 mL). The combined organic layers were washed sequentially with H₂O (2×75 mL) and brine (75 mL). Drying (MgSO₄) and concentration left a yellow oil which was chromatographed on silica gel (7% EtOAc/hexanes). The title compound (13.01 g, 78%) was obtained as a faintly yellow oil: ¹H NMR (250 MHz, CDCl₃) δ7.51 (app t, 1 H), 7.37 (d, J=8.2 Hz, 1 H), 6.86 (d, J=7.2 Hz, 1 H), 3.38 (s, 3 H), 2.49 (s, 3 H), 1.50 (s, 9 H); MS (ES) m/e 223 (M+H)⁺.

### c) Ethyl-6-[(tert-butoxycarbonyl)methylamino]-2-pyridylacetate

LDA was prepared at 0° C. under argon from diisopropylamine (19.5 mL, 139.14 mmole) and 2.5 M n-BuLi in hexanes (46.4 mL, 115.95 mmole) in dry THF (350 mL). This solution was cooled to −78° C. and a solution of 2-[(tert-butoxycarbonyl)methylamino]-6-picoline (10.31 g, 46.38 mmole) in dry THF (46 mL) was added dropwise over 10 min. Additional dry THF (2 mL) was used in transfer. The orange solution was stirred at −78° C. for 15 min, then diethyl carbonate (6.2 mL, 51.02 mmole) was added rapidly. The red solution was stirred at 78° C. for 15 min, then was quenched with half-saturated NH₄Cl (175 mL). The mixture was warmed to +5° C. and extracted with EtOAc (175 mL) then with CH₂Cl₂ (2×100 mL). The combined organics were washed with brine (100 mL), dried (MgSO₄), and concentrated. The cloudy yellow oil was chromatographed on silica gel (15% EtOAc/hexanes) to afford the title compound (10.72 g, 79%) as a light yellow oil: ¹H NMR (250 MHz, CDCl₃) δ7.51-7.63 (m, 2 H), 6.91-7.03 (m, 1 H), 4.19 (q, J=7.1 Hz, 2 H), 3.77 (s, 2 H), 3.38 (s, 3 H), 1.27 (t, J=7.1 Hz, 3 H), 1.51 (s, 9 H); MS (ES) m/e 295 (M+H)⁺.

### d) 6-[(tert-Butoxycarbonyl)methylamino]-2-pyridylethanol

A solution of 2 N LiBH₄ in THF (7 mL, 14 mmole) was added via syringe to a stirred solution of ethyl-6-[(tert-butoxycarbonyl)methylamino]-2-pyridylacetate (6.97 g, 23.7 mmole) in anhydrous THF (30 mL) under argon. The reaction was then slowly heated to reflux (initial exotherm). After 16 h at reflux, the reaction was cooled to 0° C. and carefully quenched with water (50 mL). The mixture was extracted with EtOAc (150 mL), and the organic layer was washed with brine, dried (Na₂SO₄), and concentrated. Purification by flash chromatography on silica gel (35% EtOAc/hexane) gave the title compound (5.26 g, 88%) as a clear oil: ¹H NMR (400 MHz, CDCl₃) δ7.57 (m, 2 H), 6.88 (d, J=7.2 Hz, 1 H), 4.01 (t, 2 H), 3.39 (s, 3 H), 3.00 (t, 2 H), 1.53 (s, 9 H); MS (ES) m/e 253.2 (M+H)⁺.

### e) 6-(Methylamino)-2-pyridylethanol

To 6-[(tert-butoxycarbonyl)methylamino]-2-pyridylethanol (17.9 g, 71 mmole) was added a solution of 4N HCl in dioxane (200 mL). The reaction was stirred at room temperature for 1 h (gentle gas evolution was observed) then was concentrated to dryness. The product as the hydrochloride salt solidified under vacuum. The solid was dissolved in NaCl-saturated 1.0 N NaOH solution (75 mL), and the solution was extracted with Et₂O (2×200 mL). The combined organic layers were washed with brine, dried (Na₂SO₄), and concentrated to afford the title compound (9.12 g, 85%) as a waxy solid: ¹H NMR (400 MHz, CDCl₃) δ7.37 (t, 1 H), 6.42 (d, J=7.3 Hz, 1 H), 6.27 (d, J=8.3 Hz, 1 H), 4.62 (br s, 1 H), 3.96 (t, 2 H), 2.90 (d, J=5.2 Hz, 3 H), 2.84 (t, 2 H); MS (ES) m/e 153 M+H)⁺.

### Preparation 3

### Preparation of ethyl (±)-4-(4-hydroxyphenyl)-3-phenylbutanoate

### a) Ethyl (±)-3-hydroxy-4-(4-methoxyphenyl)-3-phenylbutanoate

Anhydrous EtOAc (4.3 mL, 44 mmole) was added dropwise over 5-6 min to a solution of lithium bis(trimethylsilyl)amide (1.0 M in THF, 40 mL, 40 mmole) in dry THF (60 mL) in a flame-dried flask at −78° C. under argon. The yellow solution was stirred at −78° C. for 0.5 hr, then a solution of 2-(4-methoxyphenyl)-1-phenylethanone (Chem. Ber. 1958, 91, 755-759; 4.53 g, 20 mmole) in dry THF (20 mL) was added dropwise over 12 min. Additional THF (2 mL) was used in transfer. After 0.5 hr, The reaction was quenched with saturated NH₄Cl (120 mL) and warmed to RT. EtOAc extraction, drying (MgSO₄). concentration, and silica gel chromatography (20% EtOAc/hexanes) gave the title compound (6.13 g, 96%) as a light yellow oil: TLC R_{f} (20% EtOAc/hexanes) 0.34; MS (ES) m/e 315.2 (M+H)⁺.

### b) Ethyl (±)-4-(4-methoxyphenyl)-3-phenylbutanoate

Boron trifluoride etherate (4.8 mL, 39 mmole) was added dropwise over 3 min to a solution of ethyl (±)-3-hydroxy-4-(4methoxyphenyl)-3-phenylbutanoate(6.13 g, 19.5 mmole) and triethylsilane (6.2 mL, 39 mmole) in anhydrous CH₂Cl₂ (49 mL) at 0° C. under argon. The reaction was stirred at RT overnight, then was quenched with 5% NaHCO₃ (100 mL). The mixture was stirred briskly for 10 min, then was separated. The aqueous layer was extracted with CH₂Cl₂ (100 mL), and the combined organic layers were dried (Na₂SO₄) and concentrated. The residue was reconcentrated from hexanes (to remove CH₂Cl₂) to leave a yellow oil. This was dissolved in absolute EtOH (100 mL), and 10% Pd/C (775 mg, 1.95 mmole) was added. The mixture was shaken on a Parr apparatus at RT under H₂ (50 psi) for 2 hr, then was filtered through celite®. The filtrate was concentrated, and the residue was chromatographed on silica gel (15 % EtOAc/hexanes). The title compound (5.27 g, 91%) was obtained as a colorless oil: TLC R_{f} (15% EtOAc/hexanes) 0.40; MS (ES) m/e 299.2 (M+H)⁺.

### c) Ethyl (±)-4-(4-hydroxyphenyl)-3-phenylbutanoate

Anhydrous aluminum trichloride (4.49 g, 33.7 mmole) was added all at once to solution of ethyl (±)-4-(4methoxyphenyl)-3-phenylbutanoate(2.01 g, 6.74 mmole) and ethanethiol (2.5 mL, 33.7 mmole) in anhydrous CH₂Cl₂ (67 mL) at 0° C. under argon. The yellow solution was warmed to RT and stirred for 3 hr, then was recooled to 0° C. and quenched with cold 3 N HCl (67 mL). The mixture was stirred for 5 min, then was separated. The aqueous layer was extracted with CH₂Cl₂(2×100 mL), and the combined organic layers were dried (Na₂SO₄) and concentrated. Silica gel chromatography (25% EtOAc/hexanes) gave the title compound (1.84 g, 96%) as a colorless oil: TLC R_{f} (30% EtOAc/hexanes) 0.47; MS (ES) m/e 285.2 (M+H)⁺.

### Preparation 4

### Preparation of 2-[(2-amino-1-ethyl)amino]pyridine dihydrochloride

### a) 2-[[2-(tert-Butoxycarbonyl)amino-1-ethyl]amino]-1-oxopyridine

A mixture of N-Boc-ethylenediamine (5.83 g 36.39 mmole), 2-chloropyridine-N-oxide hydrochloride (7.25 g, g, 43.67 mmole), NaHCO₃ (15.29 g, 182 mmole), and tert-amyl alcohol (36 mL) was heated at reflux. After 47 hr, the dark brown mixture was cooled, diluted with CH₂Cl₂ (100 mL), and suction filtered. The filtrate was concentrated and the residue was reconcentrated from toluene. Silica gel chromatography (10% MeOH/CH₂Cl₂) gave the title compound (8.23 g, 89%) as a yellow solid: ¹H NMR (250 MHz, CDCl₃) δ8.16 (dd, J=6.5, 1.3 Hz, 1 H), 7.05-7.30 (m, 2 H), 6.68 (br d, J=8.6 Hz, 1 H), 6.50-6.65 (m, 1 H), 5.70-5.95 (m, I H), 3.25-3.60 (m, 4 H), 1.44 (s, 9 H); MS (ES) m/e 254 (M+H)⁺.

### b) 2-[[2-(tert-Butoxycarbonyl)amino-1-ethyl]amino]pyridine

A mixture of 2-[[2-(tert-butoxycarbonyl)amino-1-ethyl]amino]-1-oxopyridine (7.00 g, 27.64 mmole), 10% Pd/C (5.88 g, 5.53 mmole), cyclohexene (28 mL, 276.4 mmole), and isopropanol (110 mL) was heated at reflux. After 17 hr, the reaction was filtered through celite®, and the filtrate was concentrated. The yellow residue was reconcentrated from toluene, then was chromatographed on silica gel (5% MeOH/CHCl₃). The title compound (5.09 g, 78%) was obtained as a yellow oil: ¹H NMR (400 MHz, CDCl₃) δ8.05-8.12 (m, 1 H), 7.37-7.46 (m, 1 H), 6.53-6.61 (m, 1 H), 6.41 (d, J=8.3 Hz, 1 H), 5.12 (br s, 1 H), 4.86 (br s, 1 H), 3.26-3.51 (m, 4 H), 1.44 (s, 9 H); MS (ES) m/e 238 (M+H)⁺.

### c) 2-[(2-Amino-1-ethyl)amino]pyridine hydrochloride

4 N HCl/dioxane (54 mL) was added in a stream to a solution of 2-[[2-(tert-butoxycarbonyl)amino-1-ethyl]amino]pyridine (5.09 g, 21.45 mmole) in anhydrous CH₂Cl₂ (54 mL) at 0° C. under argon, then the mixture was warmed to RT. After 2 hr, the mixture was cooled to 0° C. and suction filtered. The solid was washed extensively with anhydrous Et₂O and dried in high vacuum at 40° C. to afford the title compound (4.27 g, 95%) as an off-white, somewhat hygroscopic solid: ¹H NMR (400 MHz, CD₃OD) δ7.99-8.07 (m, 1 H), 7.92-7.98 (m, 1 H), 7.19 (d, J=9.1 Hz, 1 H), 6.98-7.04 (m, 1 H), 3.76 (t, J=6.2 Hz, 2 H), 3.27 (t, J=6.2 Hz, 2 H, partially obscured by residual solvent signal), MS (ES) m/e 138 (M+H)⁺.

### Preparation 5

### Preparation of 2-[(3-hydroxy-1-propyl)amino]-4-methylpyridine-N-oxide

### a) 2-Chloro-4-methylpyridine

Sodium nitrite (13.88 g, 200 mmole) was added slowly at 0° C. to a solution of 2-amino-4-picoline (15.0 g, 139 mmole) in conc. HCl (200 mL). The reaction mixture was allowed to warm to RT and was stirred for 16 hr, then was poured onto ice (500 g). The pH was adjusted to 8.0 with conc. NH₄OH, and the mixture was extracted with ether (3×300 mL). The combined ether layers were washed sequentially with H₂O (2×200 mL) and brine (200 mL). Drying (MgSO₄) and concentration gave the title compound (10.3 g, 58%) as a faintly yellow oil: MS (ES) m/e 127.8 (M+H)⁺.

### b) 2-Chloro4-methylpyridine-N-oxide hydrochloride

A mixture of 2-chloro-4-methylpyridine (10.0 g, 78.3 mmole) and 34% peracetic acid (76.05 g, 91.0 mmole) in glacial AcOH (10 mL) was heated at 70° C. for 3 hr. The reaction mixture,was cooled, conc. HCl (35 mL) was added, and the mixture was concentrated on the rotavap. Recrystallization from n-butanol followed by trituration with ether gave the title compound (7.16 g, 51%) as a white solid: MS (ES) m/e 143.9 (M+H)⁺.

### c) 2-[(3-Hydroxy-1-propyl)amino]-4-methylpyridine-N-oxide

A mixture of 2-chloro-4-methylpyridine-N-oxide hydrochloride (7.16 g, 39 mmole), 3-aminopropanol (6.01 g, 80 mmole), and NaHCO₃ (16.8 g, 200 mmole) in tert-amyl alcohol (50 mL) was heated at reflux for 19 hr. The reaction mixture was diluted with CH₂Cl₂ (200 mL) and filtered, and the filtrate was concentrated on the rotavap. Recrystallization from CH₂Cl₂/Et₂O gave the title compound (5.41 g, 75%) as a yellow solid: TLC (15% MeOH/CH₂Cl₂) R_{f} 0.44; ¹H NMR (400, CDCl₃) δ7.92 (d, J=6.7, 1 H), 7.28 (br t, 1 H), 6.43 (s, 1 H), 6.33 (dd, J=6.6, 2.1 Hz, 1 H), 3.73 (t, J=5.7 Hz, 2 H), 3.47 (q, H=6.3 Hz, 2 H), 2.29 (s, 3 H), 1.82-1.88 (m, 2 H); MS (ES) m/e 183 (M+H)⁺.

### Preparation 6

### Preparation of 2-[(3-bromo-1-propyl)amino]pyridine-N-oxide hydrobromide

### a) 2-[(3-Bromo-1-propyl)amino]pyridine-N-oxide hydrobromide

A solution of SOBr₂ (5.0 mL, 64.5 mmole) in CH₂Cl₂ (20 mL) was added dropwise over 15-20 min to a solution of 2-[(3-hydroxy-1-propyl)amino]-4-methylpyridine-N-oxide (10.0 g, 54.87 mmole) in CH₂Cl₂ (100 mL) at 0° C. The reaction was warmed to RT and stirred for 2 hr, then Et₂O (200 mL) was added slowly. The solvents were decanted away from the gummy precipitate, and the precipitate was washed with additional CH₂Cl₂/Et₂O (several times). The resulting brownish-yellow residue solidified on standing in a refrigerator overnight. This solid was collected and washed with Et₂O to afford the title compound (15.07 g) as a yellow solid. Additional title compound (2.05 g) was obtained as white needles by concentration of the combined organic layers. The total yield of title compound was 17.89 g (96%): MS (ES) m/e 245 and 247 (M+H)⁺.

### Preparation 7

### Preparation of 2-[(5-hydroxy-1-pentyl)amino]pyridine-N-oxide

### a) 2-[(5-Hydroxy-1-pentyl)amino]pyridine-N-oxide

A suspension of 2-chloropyridine N-oxide hydrochloride (1.00 g, 6.03 mmole) and NaHCO₃ (2.53 g; 30.1 mmole) in tert-amyl alcohol (20 mL) was heated to reflux for 18 h. The reaction was cooled to RT, diluted with CH₂Cl₂, and filtered. The filtrate was concentrated to give a pale green oil. Radial chromatography (10% MeOH/CHCl₃, silica gel, 6 mm plate) gave the title compound (0.52 g) as a clear oil: ¹ H NMR (300 MHz, CDCl₃) δ8.10 (d, J=6.5 Hz, 1 H), 7.18 (t, J=7.3 Hz, 1 H), 6.85 (br s, 1 H), 6.50 (m, 2 H), 3.65 (t, J=6.2 Hz, 2 H), 3.23 (m, 2 H), 2.20 (br s, 1 H), 1.85-1.40 (m, 6H).

### Preparation 8

### Preparation of 2-[N-(tert-butoxycarbonyl)-N-methylamino]-5-pyridylethanol

### a) 5-Bromo-2-[(tert-butoxycarbonyl)amino]pyridine

A solution of 2-amino-5-bromopyridine (5.67 g, 32.7 mmole)and di-tert-butyl dicarbonate(8.57 g, 38.3 mmole) in CH₂Cl₂ (50 mL) was concentrated on the rotavap at 50° C., and the resulting residue was allowed to rotate on the rotavap at 50° C. under vacuum overnight. After 20 hr, the reaction was chromatographed on silica gel (5% MeOH/hexanes) to afford the title compound (6 g, 67% )as a white solid: MS (ES) m/e 273 (M+H)⁺.

### b) 5-Bromo-2-[N-(tert-butoxycarbonyl)-N-methylamino]pyridine

To a solution of 5-bromo-2-[(tert-butoxycarbonyl)amino]pyridine (6 g, 21.9 mmole)in dry DMF (50 mL) under nitrogen was added in portions 80% NaH (0.8 g, 26.3 mmol ) at 0° C. The reaction mixture was stirred at 0° C. for 15 min , then iodomethane (3 mL, 43.8 mmole) was added in a stream. The reaction was stirred at RT overnight, then was concentrated in vacuum. The residue was diluted with water and extracted with CH₂Cl₂. Drying (MgSO₄), concentration, and flash chromatography on silica gel (5% EtOAc/hexanes) gave the title compound (2.2 g 35%) as an oil: MS (ES) m/e 286.9 (M+H)⁺.

### c) 2-[N-(tert-Butoxycarbonyl)-N-methylamino]-5-vinylpyridine

To a solution of 5-bromo-2-[N-(tert-butoxycarbonyl)-N-methylamino]pyridine (2.2 g, 7.69 mmol ) and vinyltributyltin (3.4 mL 11.5 mmol ) in toluene at RT was added tetrakis(triphenylphosphine)palladium(0) (346 mg, 0.3 mmol). The solution was degassed under vacuum for 10 min, then was heated at reflux. After 5 hr, the reaction was cooled, concentrated in vacuum, and flash chromatographed on silica gel (5% EtOAc/hexanes) to afford the title compound (1.0 g, 65%) as a colorless oil: MS (ES) m/e 235 (M+H )⁺. Unchanged 5-bromo-2-[N-(tert-butoxycarbonyl)-N-methylamino]pyridine (0.3 g) was also recovered.

### d) 2-[N-(tert-Butoxycarbonyl)-N-methylamino]-5-pyridylethanol

To a solution of 2-[N-(tert-butoxycarbonyl)-N-methylamino]-5-vinylpyridine (1.1 g, 4.7 mmole) in dry THF (20 mL) was added borane-tetrahydrofuran complex (1.0 M in THF, 3 mL, 3 mmole) at 0° C. The reaction was heated for 1 hr, then was concentrated in vacuum. The crude product was dissolved in THF (5 mL), and NaOAc (770 mg, 9.4 mmole) was added, followed by 30% H₂O₂ (1.56 mL). The reaction was stirred at RT for 1 hr, then was partly concentrated in vacuum. The residue was treated with saturated NaCl (mL) and the mixture was extracted with CH₂Cl₂. Drying (MgSO₄), concentration, and flash chromatography on silica gel (1:1 EtOAc/hexanes) gave the title compound (230 mg, 21%) as a colorless oil: MS (ES) m/e 253 (M+H)⁺.

### Preparation 9

### Preparation of 2-[N-(3-methanesulfonyloxy-1-propyl)-N-(tert-butoxycarbonyl)amino]pyridine-N-oxide

### a) 2-[N-(3-Hydroxy-1-propyl)-N-(tert-butoxycarbonyl)amino]pyridine-N-oxide

A solution of 2-[(3-hydroxy-1-propyl)amino]pyridine-N-oxide (8.0 g, 47.6 mmole) in tert-BuOH (80 mL) was treated with di-tert-butyl dicarbonate (11.4 g, 55.3 mmole). After 18 h, the solution was concentrated and the residue was triturated with hexane. The resulting solid was dried in vacuo to give the title compound (12.5 g, 98%) as an off-white solid: MS (ES) m/e 269.3 (M+H)⁺.

### b) 2-[N-(3-Methanesulfonyloxy-1-propyl)-N-(tert-butoxycarbonyl)amino]pyridine-N-oxide

Methanesulfonyl chloride (0.17 mL, 2.20 mmole) was added dropwise to a solution of 2-[N-(3-hydroxy-1-propyl)-N-(tert-butoxycarbonyl)amino]pyridine-N-oxide (0.50 g, 1.86 mmole) and pyridine (0.23 mL, 2.84 mmole) in CHCl₃ (5 mL, dried over K₂CO₃) at 0° C. When complete by TLC, the reaction was diluted with CHCl₃, washed with ice water, dried (Na₂SO₄), and concentrated. Silica gel chromatography (10% MeOH/CHCl₃) gave the title compound (0.41 g, 64%) as a colorless oil: ¹H NMR (250 MHz, CDCl₃) δ8.25 (dd, J=6.0,1.9 Hz, 1 H), 7.25 (m, 4 H), 4.35 (t, J=6.2 Hz, 2 H), 3.75 (t, J=6.6 Hz, 2 H), 3.00 (s, 3 H), 2.00 (m, 2 H), 1.40 (s, 9 H). Unchanged 2-[N-(3-hydroxy-1-propyl)-N-(tert-butoxycarbonyl)amino]pyridine-N-oxide (0.18 g, 36%) could also be recovered from the chromatographic purification.

### Preparation 10

### Preparation of (±)-ethyl-4(4-carboxyphenyl)-3-phenylbutanoate

### a) Ethyl (±)-3-phenyl-4-[4-(trifluoromethanesulfonyloxy)phenyl]butanoate

Trifluoromethanesulfonic anhydride (1.4 mL, 8.4 mmole) was added rapidly dropwise to a solution of ethyl (±)-4-(4-hydroxyphenyl)-3-phenylbutanoate (1.84 g, 6.47 mmole) and 2,6-lutidine (1.5 mL, 12.9 mmole) in anhydrous CH₂Cl₂ (32 mL) at −78° C. under argon. After 0.5 hr, the yellow solution was warrned to RT and stirred for 1 hr. The reaction was diluted with Et₂O (150 mL) and washed sequentially with 1.0 N HCl (15 mL), 5% NaHCO₃ (15 mL), and saturated brine(15 mL). Drying (MgSO₄), concentration, and silica gel chromatography (15% EtOAc/hexanes) gave the title compound (2.62 g, 97%) as a nearly colorless oil: TLC R_{f} (20% EtOAc/hexanes) 0.55; MS (ES) m/e 417.0 (M+H)⁺.

### b) Ethyl (±)-4-(4-carboxyphenyl)3-phenylbutanoate

A mixture of ethyl (±)-3-phenyl-4-[4-(trifluoromethanesulfonyloxy)phenyl]butanoate (2.62 g, 6.29 mmole), anhydrous KOAc (2.47 g, 25.16 mmole), Pd(OAc)₂ (70.6 mg, 0.31 mmole), dppf (697.4 mg, 1.26 mmole), and anhydrous DMSO (31 mL) was purged with carbon monoxide (three evacuation/carbon monoxide purge cycles, followed by bubbling carbon monoxide through the mixture for 5 min), then was heated at 70° C. under a balloon of carbon monoxide. After 3.5 hr, the reaction was diluted with H₂O (31 mL), cooled in ice, and acidified with 1.0 N HCl (25 mL). CH₂Cl₂ extraction (2×100 mL), drying (MgSO₄), concentration, and reconcentration from toluene left a reddish-orange liquid. Silica gel chromatography (1% AcOH in 7:3 toluene/EtOAc) gave the title compound (1.78 g, 91%) as a cream-colored solid: TLC R_{f} (1% AcOH in 7:3 toluene/EtOAc) 0.47; MS (ES) m/e 313.2 (M+H)⁺.

### Preparation 11

### HPLC separation of the enantiomers of ethyl (±)-4-(4-hydroxyphenyl)-3-phenylbutanoate

### a) Ethyl (S)-(−)-4-(4-hydroxyphenyl)-3-phenylbutanoate and ethyl (R)-(+)-4-(4-hydroxyphenyl)-3-phenylbutanoate

Ethyl (±)-4-(4-hydroxyphenyl)-3-phenylbutanoate was resolved into its enantiomers using the following conditions: Daicel Chiralcel AD® column (21.2 mm×250 mm), 5% ethanol in hexane mobile phase, 15 mL/min flow rate, uv detection at 254 nm, 40 mg injection; t_{R} for ethyl (S)-(−)-4-(4-hydroxyphenyl)-3-phenylbutanoate=19.8 min.; t_{R} for ethyl (R)-(+)-4(4-hydroxyphenyl)-3-phenylbutanoate=23.0 min.

### Preparation 12

### Preparation of methyl 4-(4-hydroxyphenyl)butanoate

### a) Methyl 4-benzyloxyphenylacetate

To a suspension of K₂CO₃ (20.7 g, 150 mmoles) in acetone (50 mL) was added methyl 4-hydroxyphenyl acetate (5.0 g, 30 mmoles) and benzyl chloride (10.4 mL, 90 mmoles) and the mixture was heated to reflux. After 24 hr the mixture was cooled to RT, filtered, and concentrated. The residue was chromatographed on silica gel (10% EtOAc/hexanes) to afford the title compound (7.7 g, 100%) as a white solid: ¹H NMR (300 MHz, CDCl₃) δ7.40 (m, 5 H), 7.21 (d, J=6.6 Hz, 2 H), 6.95 (d, J=6.6 Hz, 2 H), 3.70 (s, 3 H), 3.59 (s, 2 H).

### b) 4-Benzyloxyphenethyl alcohol

To a solution of methyl 4-benzyloxyphenylacetate (1.5 g, 5.85 mmoles) in dry THF (30 mL) was added LiAIH₄ (244 mg, 6.44 mmoles) at 0° C. After 2 hr the mixture was quenched by dropwise addition of 1.0 N NaOH until white solid aluminum salts had formed. The mixture was diluted with EtOAc (100 mL), dried over MgSO₄, filtered, and concentrated to give the title compound (1.35 g, quantitative) which was used without purification. ¹H NMR (300 MHz, CDCl₃) δ7.40 (m, 5 H), 7.15 (d, J=6.6 Hz, 2 H), 6.90 (d, J=6.6 Hz, 2 H), 5.05 (s, 2 H), 3.82 (t, 2 H), 2.81 (t, 2 H).

### c) 4-Benzyloxyphenylacetaldehyde

To a solution of DMSO (0.83 mL, 11.7 mmoles) in CH₂Cl₂ (20 mL) was added oxalyl chloride (0.51 mL, 5.85 mmoles) at −78° C. After 10 min, a solution of 4-(benzloxy)phenethyl alcohol (1.35 g, 5.85 mmoles) in CH₂Cl₂ (10 mL) was added. After 30 min Et₃N (2.69 mL, 19.3 mmoles) was added and the mixture was warmed to RT. After 30 min the mixture was washed sequentially with 10 mL each H₂O, 10% HCl, and H₂O, then the resulting organic layer was dried over MgSO₄, filtered, and concentrated. The residue was used immediately in the next step without purification.

### d) Methyl 4-(4-benzyloxyphenyl)crotonate

To a solution of 4-benzyloxyphenylacetaldehyde (5.85 mmoles) in dry THF (30 mL) was added methyl (triphenylphosphoranylidene)acetate (2.4 g, 7.02 mmoles). After 18 hr the mixture was concentrated. The residue was taken up in 1:1 Et₂O/hexanes (200 mL) and filtered. The filtrate was concentrated and the residue was chromatographed on silica gel (10% EtOAc/hexanes) to afford the title compound (780 mg, 47% from b) as a yellow oil: ¹H NMR (300 MHz, CDCl₃) δ7.35 (m, 5 H), 7.05 (m, 2 H), 6.90 (m, 3 H), 5.80 (d, J=15 Hz, 1H), 5.05 (s, 2 H), 3.79 (s, 3 H), 3.47 (d, J=6.0 Hz, 2 H).

### e) Methyl 4-(4-hydroxyphenyl)butanoate

To a suspension of 10% Pd/C (113 mg) in absolute EtOH (15 mL) was added methyl 4-(4-benzyloxyphenyl)crotonate (300 mg, 1.06 mmoles). The mixture was deoxygenated (3×evacuation/N₂ purge cycles) then was charged with H₂ (50 psi). After 2 hr the H₂ was removed and the mixture was filtered through a pad of celite®. The filtrate was concentrated and the residue was chromatographed on silica gel (30% EtOAc/hexanes) to afford the title compound (180 mg, 87%) as a colorless oil: ¹H NMR (300 MHz, CDCl₃) δ7.05 (m, 2 H), 6.90 (m, 2 H), 3.68 (s, 3 H), 2.69 (t, 2 H), 2.30 (t, 2 H), 1.90 (m, 2 H).

### Preparation 13

### Preparation of methyl (±)-4-(4-hydroxyphenyl)-3-vinylbutanoate

### a) Methyl 4-(triisopropylsiloxy)phenylacetate

To a solution of methyl 4-hydroxyphenylacetate (5.0 g, 30 mmoles) and imidazole (4.08 g, 60 mmoles) in dry DMF (80 mL) was added triisopropylsilyl chloride (9.6 mL, 45 mmoles). After 18 hr the mixture was poured into H₂O (500 mL) and extracted with hexanes (3×300 mL). The combined organic layers were dried over MgSO₄, filtered, and concentrated. The residue was chromatographed on silica gel (5% EtOAc/hexanes) to give the title compound (9.03 g, 93%) as a colorless oil: ¹H NMR (300 MHz, CDCl₃) δ7.10 (d, J=6.6 Hz, 2 H), 6.80 (d, J=6.6 Hz, 2 H), 3.66(s, 3 H), 3.51 (s, 2 H), 1.23 (m, 3 H), 1.08 (d, J=7.5 Hz, 18 H).

### b) 4-(Triisopropylsiloxy)phenethyl alcohol

To a solution of methyl 4-(triisopropylsiloxy)phenylacetate (9.03 g, 28 mmoles) in dry THF (100 mL) was added LiAIH₄ (1.17 g, 30.8 mmoles) at 0° C. After 1 hr the mixture was quenched by dropwise addition of 1.0 N NaOH until white solid aluminum salts had formed. The mixture was diluted with EtOAc (100 mL), dried over MgSO₄, filtered, and concentrated to give the title compound (8.02 g, 97%) which was used without purification: ¹H NMR (300 MHz, CDCl₃) δ7.10 (d, J=6.6 Hz, 2 H), 6.80 (d, J=6.6 Hz, 2 H), 3.80 (t, 2 H), 2.79 (t, 2 H), 1.23 (m, 3 H), 1.08 (d, J=7.5 Hz, 18 H).

### c) 4-(Triisopropylsiloxy)phenylacetaldehyde

To a solution of DMSO (3.83 mL, 54 mmoles) in CH₂Cl₂ (100 mL) was added oxalyl chloride (2.36 mL, 27 mmoles) at −78° C. After 10 min, a solution of 4-(triisopropylsiloxy)phenethyl alcohol (8.02 g, 27 mmoles) in CH₂Cl₂ (25 mL) was added. After 1 hr Et₃N (12.5 mL, 89.8 mmoles) was added and the mixture was warmed to RT. After 1.5 hr the mixture was washed sequentially with 50 mL each H₂O, 10% HCl, and H₂O, then the resulting organic layer was dried over MgSO₄, filtered, and concentrated. The residue was used immediately in the next step without purification.

### d) Methyl 4-[(4triisopropylsiloxy)phenyl]crotonate

To a solution of 4-(triisopropylsiloxy)phenylacetaldehyde (27 mmoles) in dry benzene (100 mL) was added methyl (triphenylphosphoranylidene)acetate (18.1 g, 54 mmoles). After 96 hr the mixture was concentrated. The residue was taken up in Et₂O (500 mL) and filtered. The filtrate was concentrated and the residue was chromatographed on silica gel (2:1 hexanes/CH₂Cl₂) to afford the title compound (3.39 g, 36% from b) as a yellow oil: ¹H NMR (300 MHz, CDCl₃) δ7.09 (m, 1 H), 6.99 (d, J=6.6 Hz, 2 H), 6.79 (d, J=6.6 Hz, 2 H), 5.78 (d, J=15 Hz, 1 H), 3.71 (s, 3 H), 3.42 (d, J=7.1 Hz, 2 H), 1.23 (m, 3 H), 1.08 (d, J=7.5 Hz, 18 H).

### e) Methyl (±)-4-[(4-triisopropylsiloxy)phenyl]-3-vinylbutanoate

To a suspension of CuBr-DMS complex (647 mg, 3.0 mmoles) in dry THF (10 mL) was added vinyl magnesium bromide (6.0 mL, 6.0 mmoles) dropwise at −78° C. After 15 min, a solution of methyl 4-[(4-triisopropylsiloxy)phenyl]crotonate (350 mg. 1.0 mmoles) in dry THF (3 mL) was added dropwise. After 1.5 hr the mixture was quenched with H₂O (10 mL) and extracted with EtOAc (3×20 mL). The combined organic layers were dried over MgSO₄, filtered, and concentrated. The residue was chromatooraphed on silica gel (3:1 hexanes/CH₂Cl₂) to give the title compound (224 mg, 59%) as a yellow oil: ¹H NMR (300 MHz, CDCl₃) δ6.99 (d, J=6.6 Hz, 2 H), 6.79 (d, J=6.6 Hz, 2 H), 5.69 (m, 1 H), 4.95 (m, 2 H), 3.60 (s, 3 H), 2.80 (m, 1 H), 2.59 (m, 2 H), 2.32 (m, 2 H), 1.23 (m, 3 H), 1.08 (d, J=7.5 Hz, 18 H).

### f) Methyl (±)-4-(4-hydroxyphenyl)-3-vinylbutanoate

To a solution of methyl (±)-4-[(4-triisopropylsiloxy)phenyl]-3-vinylbutanoate (224 mg, 0.59 mmoles) in dry THF (5 mL) was added a solution of TBAF in THF (1.0 M, 0.65 mL, 0.65 mmoles). After 1 hr the mixture was diluted with H₂O (10 mL) and extracted with EtOAc (3×20 mL). The combined organic layers were dried over MgSO₄, filtered, and concentrated. The residue was chromatographed on silica gel (30% EtOAclhexane) to give the title compound (92.5 mg, 71%) as a yellow oil: ¹H NMR (300 MHz, CDCl₃) δ7.00 (d, J=6.6 Hz, 2 H), 6.74 (d, J=6.6 Hz, 2 H), 5.70 (m, 1 H), 4.99 (m, 2 H), 4.75 (bs, 1 H), 3.62 (s, 3 H), 2.80 (m, 1 H), 2.59 (m, 2 H), 2.32 (m, 2 H).

### Preparation 14

### Preparation of ethyl (±)-4-(4-hydroxyphenyl)-3-(pyridin-2-yl)butanoate

### a) 4-Benzyloxy-N-methoxy-N-methylphenylacetamide

To a suspension of N,O-dimethylhydroxylamine hydrochloride (761 mg, 7.8 mmoles) in dry toluene (20 mL) was added trimethylaluminum (7.8 mL, 7.8 mmoles) at RT. After 1 hr methyl 4-(benzyloxy)phenylacetate (1.0 g, 3.9 mmoles) was added and the mixture was heated to reflux. After 2 hr the mixture was cooled to RT and stirred for 18 hr, then was quenched by the slow addition of 10% HCl (20 mL) and extracted with EtOAc (3×30 mL). The combined organic layers were dried over MgSO₄, filtered, and concentrated. The residue was chromatographed on silica gel (75% EtOAc/hexanes) to give the title compound (473 mg, 43%) as an orangish solid: MS (ES) m/e 286 (M+H)⁺.

### b) 2-[4-(Benzyloxy)phenyl]-1-(pyridin-2-yl)ethanone

To a solution of 2-bromopyridine (0.08 mL, 0.8 mmoles) in dry THF (2 mL) was added t-BuLi (0.94 mL, 1.6 mmoles) at −78° C. After 10 min, a solution of 4-benzyloxy-N-methoxy-N-methylphenylacetamide (115 mg, 0.4 mmoles) in dry THF (2 mL) was added. The mixture was allowed to warm as the bath warmed. After 18 hr the mixture was quenched with saturated NH₄Cl (20 mL) and extracted with EtOAc (3×20 mL). The combined organic layers were dried over MOSO₄, filtered, and concentrated. The residue was chromatographed on silica gel (15% EtOAc/hexanes) to give the title compound (80 mg, 66%) as an orangish solid: MS (ES) m/e 304 (M+H)⁺.

### c) Ethyl (±)-4-[4-(benzyloxy)phenyl]-3-(pyridin-2-yl)crotonate

To a suspension of NaH (21 mg, 0.53 mmoles) in dry THF (2 mL) was added triethyl phosphonoacetate (0.11 ml, 0.53 mmoles) dropwise at RT. After 10 min, a solution of 2-[4-(benzyloxy)phenyl]-1-(pyridin-2-yl)ethanone (80 mg, 0.26 mmoles) in dry THF (2 mL) was added dropwise. After 4 hr the mixture was concentrated. The residue was chromatographed on silica gel (30% EtOAc/hexanes) to give the title compound (82 mg, 84%) as a mixture of olefin isomers: MS (ES) m/e 374 (M+H)⁺.

### d) Ethyl (±)-4(4-hydroxyphenyl)-3-(pyridin-2-yl)butanoate

To a suspension of 10% Pd/C (69 mg) in 1:1 EtOAc/i-PrOH (10 mL) was added ethyl (±)-4-[4-(benzyloxy)phenyl]-3-(pyridin-2-yl)crotonate (243 mg, 0.65 mmoles). The mixture was deoxygenated (3×evacuation/N₂ purge cycles) then was charged with H₂ (50 psi). After 4 hr the H₂ was removed and the mixture was filtered through a pad of celite®. The filtrate was concentrated to afford the title compound as an oil (90 mg, 49%) which was used without purification: ¹H NMR (300 MHz, CDCl₃) δ8.55 (d, 1 H), 7.48 (t, 1 H), 7.08 (m, 1 H), 6.95 (m, 3 H), 6.80 (m, 3 H), 3.98 (q, 2 H), 3.55 (m, 1 H), 2.90 (m, 2 H), 2.62 (m, 2 H), 1.09 (t, 3 H).

### Preparation 15

### Preparation of methyl (±)-4-(4-hydroxyphenyl)-3-(oxazol-2-yl)butanoate

### a) Methyl 3-(benzyloxycarbonyl)-3-butenoate

Diisopropyl azodicarboxylate (32.8 mL, 166 mmole) was added to a solution of methyl 3-carboxy-3-butenoate (20 g, 139 mmole), benzyl alcohol (17.2 mg, 166 mmole), and triphenylphosphine (43.7 g, 166 mmole) in anhydrous THF (500 mL) at 0° C. The mixture was allowed to warm as the bath warmed to RT. After 3 hr the mixture was concentrated and the residue was chromatographed on silica gel (10% EtOAc/hexanes) The title compound (29.46 g, 91%) was obtained as a colorless oil: ¹H NMR (300 MHz, CDCl₃) δ7.35 (m, 5 H), 6.48 (s, 1 H), 5.71 (s, 1 H), 5.20 (s, 2 H), 3.63 (s, 3 H), 3.37 (s, 2 H).

### b) Methyl (±)-4-(4-methoxyphenyl)-3-carboxybutanoate

A solution of 4-bromoanisole (3.35 mL, 26.7 mmoles), methyl 3-(benzyloxycarbonyl)-3-butenoate (12.5 g, 53.4 mmoles), Pd(OAc)₂ (599 mg, 2.67 mmoles), P(o-tolyl)₃ (1.63 g, 5.34 mmoles), and (i-Pr)₂NEt (9.3 mL, 53.4 mmoles) in propionitrile (100 mL) was deoxygenated (3×evacuation/N₂ purge cycles) then was heated to reflux. After 24 hr the mixture was concentrated, and the residue was chromatographed on silica gel (15% EtOAc/hexanes) to give a yellow oil. The oil was taken up in 20% EtOAc/hexanes (100 mL), and the solution was allowed to stand at RT. After 18 hr the mixture was filtered and the filtrate was concentrated to give the title compound as a mixture of olefin isomers. This was used immediately in the next step.

To a suspension of 10% Pd/C (2.8 g) in 1:1 EtOAc/i-PrOH (100 mL) was added the above olefin mixture. The mixture was deoxygenated (3×evacuation/N₂ purge cycles) then was charged with H₂ (50 psi). After 4 hr the H₂ was removed and the mixture was filtered through a pad of celite®. The filtrate was concentrated to afford the title compound (5.81 mg, 86% from 4-bromoanisole) as a yellow oil: ¹H NMR (300 MHz, CDCl₃) δ7.09 (d, J=6.8 Hz, 2 H), 6.81 (d, J=6.8 Hz, 2 H), 3.78 (s, 3 H), 3.64 (s, 3 H), 3.08 (m, 2 H), 2.68 (m, 2 H), 2.40 m, 1 H).

### c) Methyl (±)-4-(4methoxyphenyl)-3-[(2,2-dimethoxyethyl)aminocarbonyl]butanoate

To a solution of methyl (±)-4-(4-methoxyphenyl)-3-carboxybutanoate (300 mg, 1.19 mmoles) in CH₂Cl₂ (5 mL) was added 1,1′-carbonyl diimidazole (289 mg, 1.78 mmoles). After 1 hr aminoacetaldehyde dimethyl acetal (0.2 mL, 1.78 mmoles) was added. After 72 hr the mixture was concentrated. The residue was chromatographed on silica gel (50% EtOAc/hexanes) to give the title compound (287 mg, 71 %) as a clear oil: MS (ES) m/e 340 (M+H)⁺.

### d) Methyl (±)-4-(4-methoxyphenyl)-3-(oxazol-2-yl)butanoate

To a solution of methyl 4(4-methoxyphenyl)-3-[(2,2dimethoxyethyl)aminocarbonyl]butanoate (287 mg, 0.85 mmoles) in THF (5 mL) was added 6.0 N HCl (5 mL). After 1 hr the mixture was extracted with EtOAc (3×10 mL). The combined organic layers were dried over MgSO₄, filtered, and concentrated. The residue was taken up in CH₂Cl₂ (5 mL) and added to a solution of PPh₃ (267 mg, 1.02 mmoles), I₂ (259 mg, 1.02 mmoles), and Et₃N (0.24 mL, 1.02 mmoles) in CH₂Cl₂ (5 mL). After 18 hr the mixture was concentrated. The residue was chromatographed on silica gel (50% EtOAc/hexanes) to give the title compound (95 mg, 41%) as a yellow oil: MS (ES) m/e 276 (M+H)⁺.

### e) Methyl (±)-4-(4-hydroxyphenyl)-3-(oxazol-2-yl)butanoate

To a solution of methyl (±)-4-(4-methoxyphenyl)-3-(oxazol-2-yl)butanoate (314 mg, 1.14 mmoles) in CH₂Cl₂ (5 mL) was added BBr₃ (3.42 mL, 3.42 mmoles) at −20° C. After 1 hr the mixture was carefully quenched with 10% HCl in MeOH (10 mL), and the solution was allowed to warm to RT. After 18 hr the mixture was concentrated. The residue was taken up in saturated NaHCO₃ (20 mL) and extracted with Et₂O (3×20 mL). The combined organic layers were dried over MgSO₄, filtered and concentrated. The residue was chromatographed on silica gel (50% EtOAc/hexanes) to give the title compound (163 mg, 55%) as a yellow oil: MS (ES) m/e 262 (M+H)⁺.

### Preparation 16

### Preparation of ethyl (±)-4-(4-hydroxyphenyl )-3-(thiazol-2-yl)butanoate

### a) 2-[4-(Benzyloxy)phenyl]-1-(thiazol-2-yl)ethanone

To a solution of n-BuLi (0.98 mL, 2.44 mmoles) in dry Et₂O (5 mL) was added 2-bromothiazole (0.21 mL, 2.34 mmoles) dropwise at −78° C. After 20 min methyl 4-(benzyloxy)phenylacetate (0.5 g, 1.95 mmoles) in dry Et₂O (5 mL) was added dropwise. After 1 hr the mixture was quenched with saturated NH₄Cl (10 mL), warmed to RT, and extracted with Et₂O (3×20 mL). The combined organic layers were dried over MgSO₄, filtered, and concentrated. The residue was chromatographed on silica gel (20% EtOAc/hexanes) to give the title compound (485 mg, 80%) as a brownish-yellow solid. MS (ES) m/e 310 (M+H)⁺.

### b) Ethyl (±)-4-[4-(benzyloxy)phenyl]-3-(thiazol-2-yl)crotonate

To a suspension of NaH (111 mg, 2.78 mmoles) in dry THF (5 mL) was added triethyl phosphonoacetate (0.56 mL, 2.78 mmoles) dropwise at RT. After 15 min, a solution of 2-[4-(benzyloxy)phenyl]-1-(thiazol-2-yl)ethanone (430 mg, 1.39 mmoles) in dry THF (5 mL) was added dropwise. After 6 hr the mixture was quenched with saturated NH₄Cl (10 mL) and extracted with EtOAc (3×20 mL). The combined organic layers were dried over MgSO₄, filtered, and concentrated. The residue was chromatographed on silica gel (20% EtOAc/hexanes) to give the title compound (356 mg, 67%) as a mixture of olefin isomers: MS (ES) m/e 380 (M+H)⁺.

### c) Ethyl (±)-4-(4-hydroxyphenyl)-3-(thiazol-2-yl)butanoate

To a suspension of 10% Pd/C (100 mg) in absolute EtOH (5 mL) was added ethyl (±)-4-[4-(benzyloxy)phenyl]-3-(thiazol-2-yl)crotonate (356 mg, 0.94 mmoles). The mixture was deoxygenated (3×evacuation/N₂ purge cycles) then was charged with H₂ (50 psi). After 4 hr the H₂ was removed and the mixture was filtered through a pad of celite®. The filtrate was concentrated. The reaction was repeated three times. The residue was chromatographed on silica gel (35% EtOAc/hexanes) to afford the title compound (155 mg, 57%) as an oil: MS (ES) m/e 292 (M+H)⁺.

### Preparation 17

### Preparation of ethyl (±)-4-(4-hydroxyphenyl)-3-methylbutanoate

### a) Ethyl (±)-4-(4-methoxyphenyl)-3-methylcrotonate

According to the procedure of Preparation 16(b), except substituting 4methoxyphenylacetone for the of 2-[4-(benzyloxy)phenyl]-1-(thiazol-2-yl)ethanone, the title compound (5.2 g, 74%) was prepared: ¹H NMR (300 MHz, CDCl₃) δ7.08 (d, J=8.7 Hz, 2 H), 6.85 (d, J=8.7 Hz, 2 H), 5.66 (narrow m, 1 H), 4.14 (q, J=7.1 Hz, 2 H), 3 H), 3.37 (s, 2 H), 2.12 (d, J=1.2 Hz, 3 H), 1.27 (t, J=7.1 Hz, 3 H).

### b) Ethyl (±)-4-(4-methoxyphenyl)-3-methylbutanoate

According to the procedure of Preparation 16(c), except substituting ethyl (±)-4-(4-methoxyphenyl)-3-methylcrotonate for the ethyl (±)-4-[4-(benzyloxy)phenyl]-3-(thiazol-2-yl)crotonate, the title compound (5.1 g, 97%) was prepared as a colorless oil: ¹H NMR (300 MHz, CDCl₃) δ7.07 (d, J=8.5 Hz, 2 H), 6.83 (d, J=8.5 Hz, 2 H), 4.11 (q, J=7.1 Hz, 2 H), 3.79 (s, 3 H), 2.00-2.60 (m, 5 H), 1.25 (t, J=7.1 Hz, 3 H), 0.93 (d, J=6.3 Hz, 3 H).

### c) Ethyl (±)-4-(4-hydroxyphenyl)-3-methylbutanoate

According to the procedure of Preparation 15(e), except substituting ethyl (±)-4(4-methoxyphenyl)-3-methylbutanoate for the methyl (±)-4-(4-methoxyphenyl)-3-(oxazol -2-yl)butanoate, the title compound (3.2 g, 70%) was prepared as a yellow oil: ¹H NMR (250 MHz, CDCl₃) δ7.00 (d, 2 H), 6.76 (d, 2 H), 5.95-6.15 (m, 1 H), 4.13 (q, 2 H), 2.05-2.60 (m, 5 H), 1.25 (t, 3 H), 0.93 (d, 3 H).

### Preparation 18

### Preparation of methyl 4-(4-methoxyphenyl)crotonate

### a) 4-Methoxyphenylacetaldehyde

A solution of 4-methoxyphenethyl alcohol (114 g, 7.49 mmole) in CH₂Cl₂ (30 mL) was added dropwise to a suspension of PCC (2.45 g, 11.37 mmole) and NaOAc (1.85 g, 22.55 mmole) in CH₂Cl (50 mL) at 0° C. under argon. After 1 hr, the mixture was filtered, and both celite® and activated charcoal were added to the filtrate. This mixture was filtered, and the filtrate was concentrated on the rotavap. The residue was dissolved in Et₂O, and both MgSO₄ and activated charcoal were added. Filtration and concentration gave the title compound (1.1 g, 98%) as a colorless oil. This material was used immediately in the next step without further purification.

### b) Methyl 4(4-methoxyphenyl)crotonate

A solution of 4-methoxyphenylacetaldehyde (1.1 g, 7.33 mmole) and methyl (triphenylphosphoranylidene)acetate (2.99 g, 8.94 mmole) in THF (50 mL) was stirred at RT overnight, then was concentrated in vacuum. The residue was dissolved in Et₂O, and the solution was treated with celite® and activated charcoal. Filtration, concentration, and silica gel chromatography (5% EtOAc/hexanes) gave the title compound (0.5 g, 33%): ¹H NMR (300 MHz, CDCl3) δ7.00-7.20 (m, 3 H), 6.85 (d, J=8.6 Hz, 2 H), 5.79 (d, J=15.5 Hz, 1 H), 3.79 (s, 3 H), 3.71 (s, 3 H), 3.46 (d, J=6.7 Hz, 2 H).

### c) Methyl 4-(4-hydroxyphenyl)crotonate

BBr₃ (1.0 M in CH₂Cl₂, 4.0 mL, 4.0 mmole) was added dropwise to a solution of methyl 4-(4-methoxyphenyl)crotonate (0.75 g, 3.64 mmole) in CH₂Cl₂ (30 mL) at 0° C. under argon. The reaction was stirred at 0° C. for 2 hr, then additional BBr₃ (1.0 M in CH₂Cl₂, 1.0 mL, 1.0 mmole) was added. After another 1 hr, the reaction was quenched carefully by slow addition of MeOH. The solution was concentrated, and the residue was reconcentrated from MeOH (2×). The resulting residue was flash chromatographed on silica gel (1% MeOH/CH₂Cl₂) to afford the title compound (0.46 g, 66%): ¹H NMR (300 MHz, CDCl3) 86.95-7.25 (m, 3 H), 6.80 (d, J=8.4 Hz, 2 H), 5.82 (d, J=15.6 Hz, 1 H), 5.08 (s, 1 H), 3.75 (s, 3 H), 3.48 (d, J=6.8 Hz, 2 H).

### Preparation 19

### Preparation of methyl (±)-4-(4-hydroxyphenyl)-3-(thiophen-2-yl)butanoate

### a) Ethyl (±)-3-(4-methoxyphenyl)-2-(thiophen-2-yl)propionate

Lithium hexamethyldisilazide (1.0 M in THF, 14 mL, 14.0 mmole) was added to a solution of ethyl 2-thiopheneacetate (2.268 g, 13.32 mmole) in dry THF (10 mL) at −78° C. under argon. After 1 hour, 4-methoxybenzyl chloride (2.0 mL, 14.75 mmole) was added. The reaction was kept at −78° C. for another 15 min, then was allowed to warm to RT. After 18 hours, the reaction was diluted with EtOAc and the solution was washed with 1.0 N HCl (2×) followed by 1.0 N NaHCO₃ (2×). Drying (MgSO₄), concentration, and flash chromatography on silica gel (gradient: 5% EtOAc/hexanes, then 10% EtOAc/hexanes, then 20% EtOAc/hexanes) gave the title compound (2.71 g, 66%) as a clear colorless oil: ¹H NMR (300 MHz, CDCl₃) δ7.16-7.14 (m, 1 H), 7.04 (d, J=8.7 Hz, 2 H), 7.02-6.87 (m, 2 H), 6.76 (d, J=8.7 Hz, 2 H), 4.14-4.02 (m, 3 H), 3.71 (s, 3 H), 3.30 (dd, J=13.6, 8.9 Hz, 1 H), 3.04 (dd, J=13.7, 6.7 Hz, 1 H), 1.12 (t, J=7.2, 3 H).

### b) (±)-1-Diazo-4(4-methoxyphenyl)-3-(thiophen-2-yl)-2-butanone

1.0 N NaOH (10 mL, 10 mmole) was added to a solution of ethyl (±)-3-(4-methoxyphenyl)-2-(thiophen-2-yl)propionate (2.71 g, 8.84 mmole) in MeOH (10 mL), and the resulting bright yellow mixture was further diluted with MeOH and THF to dissolve a precipitated oil. After 18 hr at RT, the reaction was neutralized with 1.0 N HCl (10 mL), and the volatile organics were removed in vacuum. The remaining aqueous layer was acidified with 1.0 N HCl and extracted with EtOAc. The combined organic layers were dried (MgSO₄), filtered and concentrated in vacuum. The residue was dissolved in excess SOCl₂, and the solution was heated at reflux for 1 hr. The reaction was concentrated in vacuum and the residue was reconcentrated from toluene (2×). The resulting residue was dissolved in THF. and diazomethane, generated from Diazald (2.0077 g, 9.4 mmole), was added at RT. More diazomethane from Diazald (1.4420 g, 6.7 mmole) was added, and the reaction was left stirring at RT overnight. The resulting orange reaction was concentrated in vacuum and the residue was adsorbed onto silica gel. This was loaded onto a dry silica gel column. Flash chromatography (gradient: 5% EtOAc/hexanes, then 10% EtOAc/hexanes, then 20% EtOAc/hexanes) gave the title compound (707.6 mz, 30%) as an oil: ¹H NMR (300 MHz, CDCl₃) δ7.25-7.19 (m, 1 H), 7.03 (d, J=8.6 Hz, 2 H), 6.94-6.85 (m, 2 H), 6.77 (d, J=8.7 Hz, 2 H), 5.18 (s, 1 H), 3.75 (s, 3 H), 3.41 (dd, J=13.8, 7.9 Hz, 1 H), 3.00 (dd, J=13.8, 7.2 Hz, 1 H).

### c) Methyl (±)-4-(4-methoxyphenyl)-3-(thiophen-2-yl)butanoate

A solution of silver benzoate (744.2 mg, 3.25 mmole) in triethylamine (3 mL, 21.6 mmole) was added to a solution of (±)-1-diazo-4-(4-methoxyphenyl)-3-(thiophen-2-yl)-2-butanone (707.6 mg, 2.47 mmole) in MeOH (20 mL) at RT. Gas evolution was observed, and the reaction mixture became black in color. After 30 min, the reaction was heated to reflux. After 1 hr at reflux, the reaction was filtered through celite® and the filtrate was concentrated in vacuum. The residue was adsorbed onto silica gel and was loaded onto a dry silica gel column. Flash chromatography (gradient: 5% EtOAc/hexanes, then 10% EtOAc/hexanes) gave the title compound (453.4 mg, 48.0%) as a light yellow oil: ¹H NMR (300 MHz, CDCl₃) δ7.16-7.14 (m, 1 H), 7.04 (d, J=8.5 Hz, 2 H), 6.91-6.89 (m, 1 H), 6.81 (d, J=8.5 Hz, 2 H), 6.77-6.76 (m, 1 H), 3.78 (s, 3 H), 3.74-3.72 (m, 1 H), 3.61 (s, 3 H), 2.97-2.92 (m, 2 H), 2.71-2.65 (m, 2 H).

### d) Methyl (±)-4-(4-hydroxyphenyl)-3-(thiophen-2-yl)butanoate

Boron tribromide (1.0 M in CH₂Cl₂, 8 mL, 8 mmole) was added to a solution of methyl (±)-4-(4-methoxyphenyl)-3-(thiophen-2-yl)butanoate (453.4 mg, 1.56 mg) in CH₂Cl₂ (10 mL) at 0° C. under argon. After 1 hr, the reaction was quenched with absolute MeOH and concentrated in vacuum. Reconcentration from toluene (several times) followed by drying in high vacuum gave the title compound (449.6 mg, quantitative) as an oil: ¹H NMR (300 MHz, CDCl₃) δ7.30-7.14 (m, 2 H), 7.04 (d, J=8.2 Hz, 2 H), 6.95-6.89 (m, 1 H), 6.74 (d, J=8.4 Hz, 2 H), 6.14 (br s, 1 H), 3.74-3.71 (m, 1 H), 3.62 (s, 3 H), 2.95-2.89 (m, 2 H), 2.72-2.66 (m, 2 H).

### Preparation 20

### Preparation of ethyl 2-[N-benzyl-N-(4-hydroxybenzyl)amino]acetate

### a) Ethyl2-[N-benzyl-N-(4-methoxybenzyl)amino]acetate

To a solution of 4-methoxybenzyl chloride (1.00 mL, 7.38 mmole) in DMF (14 mL) at 0° C. was added ethyl 2-benzylaminoacetate (1.20 mL, 6.40 mmole) followed by NaH (0.38 g, 60% dispersion in oil, 9.50 mmole). The ice bath was removed and the reaction was allowed to stir at RT for 18 h. The reaction was quenched by pouring into saturated NaHCO₃, and the mixture was extracted with EtOAc. The combined organic extracts were dried over Na₂SO₄ and concentrated to give a yellow oil. Radial chromatography (10% EtOAc/hexanes, silica gel, 6 mm plate) gave the title compound (0.40 g) as a clear oil: MS (ES) m/e 314.1 (M+H)⁺.

### b) Ethyl 2-[N-benzyl-N-(4-hydroxybenzyl)amino]acetate

A solution of ethyl 2-[N-benzyl-N-(4-methoxybenzyl)amino]acetate (0.40 g, 1.27 mmole) in CH₂Cl₂ (2 mL) was added dropwise to a solution of BBr₃ (3.80 mL, 1.0 M in CH₂Cl₂, 3.80 mmole) at 0° C. After 1 h at 0° C., the reaction was carefully quenched with MeOH (2 mL). The solvent was removed under reduced pressure and the residue was azeotroped from MeOH (2×). Radial chromatography (30% EtOAc/hexanes, silica gel, 6 mm plate) gave the title compound (0.19 g) as a white solid: MS (ES) m/e 300.1 (M+H)⁺.

### Preparation 21

### Preparation of methyl 2-[N-(4-hydroxybenzyl)-N-phenylamino]acetate

### a) Methyl 2-[N-(4-methoxybenzyl)-N-phenylamino]acetate

To a solution of methyl 2-(phenylamino)acetate hydrochloride (0.19 g, 0.96 mmole) in DMF (3 mL) was added 4-methoxybenzyl chloride (0.52 mL, 3.84 mmole) followed by NaH (0.11 g, 60% dispersion in oil, 2.75 mmole). After 18 h at RT, the reaction was poured into saturated NaHCO₃, and the mixture was and extracted with EtOAc. The combined organic extracts were washed with 50% brine, dried over Na₂SO₄, and concentrated to give a yellow oil. Radial chromatography (20% EtOAc/hexane, silica gel, 6 mm plate) gave the title compound (0.13 g) as a clear oil: MS (ES) m/e 286.1 (M+H)⁺.

### b) Methyl 2-[N-(4-hydroxybenzyl)-N-phenylamino]acetate

A solution of methyl 2-[N-(4-methoxybenzyl)-N-phenylamino]acetate (0.13 g, 0.47 mmole) in CH₂Cl₂ was added dropwise to a solution of BBr₃ (1.40 mL, 1.0 M in CH₂Cl₂, 1.40 mL) at 0° C. After 45 min at 0° C., the reaction was carefully quenched by the addition of MeOH (2 mL). The solvent was removed under reduced pressure and the residue was azeotroped from MeOH (2×). The residue was dissolved in saturated NaHCO₃, and the solution was extracted with EtOAc. The combined organic extracts were dried over Na₂SO₄ and the solvent was removed under reduced pressure to give a pale yellow oil Radial chromatography (30% EtOAc/hexane, silica gel, 2 mm plate) gave the title compound (39 mg) as a pale yellow solid: MS (ES) m/e 272.2 (M+H)⁺.

### Preparation 22

### Preparation of methyl 2-[(4-hydroxy-2-methoxybenzyl)amino]acetate

### a) Methyl 2-[(4-hydroxy-2-methoxybenzyl)amino]acetate

To a suspension of 4-hydroxy-2-methoxybenzaldehyde (2.00 g, 13.1 mmole) and glycine methyl ester hydrochloride (6.60 g, 52.6 mmole) in dry MeOH (100 mL) was added 4 Å molecular sieves (ca. 2 g) and NaBH₃CN (0.83 g. 13.2 mmole). After 18 hr at RT, the reaction mixture was filtered through a bed of celite® and the solvent was removed under reduced pressure to leave a white residue. Flash chromatography on silica gel (10% MeOH/CHCl₃) gave the title compound (1.27 g) as a clear oil: MS (ES) m/e 226.0 (M+H)⁺.

### Preparation 23

### Preparation of methyl 2-(4-hydroxy-2-phenoxyphenyl)acetate

### a) 2-(4-Methoxy-2-phenoxyphenyl)-1-morpholin-4-ylethan-1-thione

According to the procedure of Harris, T. W., et al. (J. Med. Chem. 1982, 25(7), 855-858), 4-methoxy-2-phenoxyacetphenone (1.69 g, 6.98 mmole), sulfur (0.36 g, 11.2 mmole), and morpholine (0.98 mL, 11.2 mmole) were reacted to give the title compound (1.24 g) as a white solid: MS (ES) m/e 344.0 (M+H)⁺.

### b) 2-(4-Methoxy-2-phenoxyphenyl)acetic acid

To a solution of 2-(4-methoxy-2-phenoxyphenyl)-1-morpholin-4-ylethan-1-thione (0.35 g, 1.02 mmole) in i-PrOH (15 mL) and H₂O (15 mL) was added KOH (0.57 g, 10.2 mmole). The reaction was heated at reflux for 18 hr, then was cooled to RT, diluted with H₂O, and washed with Et₂O. The aqueous layer was acidified to pH≈4 with conc. HCl and was extracted with CHCl₃. The combined extracts were dried over MgSO₄ and concentrated to give the title compound (0.22 g) as a white solid. This was used without further purification: MS (ES) m/e 259.0 (M+H)⁺.

### c) Methyl 2-(4-methoxy-2-phenoxyphenyl)acetate

To a solution of 2-(4-methoxy-2-phenoxyphenyl)acetic acid (0.22 g, 0.85 mmole) in MeOH (10 mL) was added conc. H₂SO₄ (1 drop). The reaction was heated at reflux for 18 hr, then was allowed to cool to RT. The bulk of the MeOH was removed under reduced pressure, and the remaining solution was poured into saturated NaHCO₃. The aqueous layer was extracted with EtOAc, and the combined organic extracts were washed with brine and dried over Na₂SO₄. The solvent was removed under reduced pressure to give the title compound (0.22 g) as a pale yellow oil. This was used without further purification: MS (ES) m/e 273.0 (M+H)⁺.

### d) Methyl 2-(4-hydroxy-2-phenoxyphenyl)acetate

To a solution of BBr₃ (1.0 M in CH₂Cl₂, 4.0 mL, 4 mmole) at 0° C. was added dropwise a solution of methyl 2-(4-methoxy-2-phenoxyphenyl)acetate (0.22 g, 0.81 mmole) in CH₂Cl₂ (1 mL). After 20 min, the solvent was removed under reduced pressure and the residue was azeotroped from MeOH (2×). The residue was then dissolved in saturated NaHCO₃, and the solution was extracted with EtOAc. The combined extracts were dried over Na₂SO₄ and concentrated to give the title compound (0.19 g) as a pale yellow oil. This was used without further purification: MS (ES) m/e 259.0 (M+H)⁺.

### Preparation 24

### Preparation of methyl 2-(2-phenoxy-4-hydroxy)phenylbutanoate

### a) 2-(2-Phenoxy-4-methoxy)phenylethan-1-ol

To a solution of 2-(4-methoxy-2-phenoxyphenyl)acetic acid (0.24 g, 0.93 mmole) in THF (5 mL) at 0° C. was added lithium aluminum hydride (0.11 g, 2.79 mmole). After 1 hr at 0° C., the reaction was diluted with toluene (10 mL), and NaF (0.47 g) and H₂O (0.15 mL) were added sequentially. The mixture was stirred vigorously at 0° C. for 30 min. The resulting precipitate was removed by filtration and rinsed with Et₂O. The filtrate was concentrated to give the title compound (0.16 g) as a clear oil. The material was used without further purification: ¹H NMR (300 MHz, CDCl₃) δ7.30 (m, 3 H), 7.08 (t, J=7.4 Hz, 1 H), 6.95 (d, J=7.6 Hz, 2 H), 6.66 (dd, J=8.4, 2.5 Hz, 1 H), 6.45 (d, J=2.6 Hz, 1 H), 3.82 (q, J=6.4 Hz, 2 H), 3.73 (s, 3 H). 2.85 (t, J=6.6 Hz, 2 H).

### b) 2-(2-Phenoxy-4-methoxy)phenylacetaldehyde

Oxalyl chloride (0.06 mL, 0.69 mmole) was added to a solution of DMSO (0.09 mL, 1.27 mmole) in CH₂Cl₂ (1.2 mL) at −78° C. After 10 min, a solution of 2-(2-phenoxy-4-methoxy)phenylethan-1-ol (0.16 g, 0.64 mmole) in CH₂Cl₂ (1.2 mL) was added. The reaction was stirred at −78° C. for an additional 1 hr. then Et₃N (0.27 mL, 1.94 mmole) was added, and the −78° C. bath was removed. After an additional 20 min, the reaction was diluted with CH₂Cl₂ and washed sequentially with 1.0 N HCl, saturated NaHCO₃, and brine then was dried over Na₂SO₄. The solvent was removed under vacuum to give the title compound (0.13 g) as a pale yellow oil. The material was used without further purification: ¹H NMR (300 MHz, CDCl₃) δ9.71 (t, J=1.9 Hz, 1 H), 7.30 (m, 2 H), 7.10 (m, 2 H), 6.95 (d, J=7.7 Hz, 2 H), 6.66 (dd, J=8.4, 2.5 Hz, 1 H), 6.45 (d, J=2.5 Hz, 1 H), 3.71 (s, 3 H), 3.64 (s, 2 H).

### c) Methyl 2-(2-phenoxy-4-methoxy)phenylbut-2-enoate

A solution of 2-(2-phenoxy-4-methoxy)phenylacetaldehyde (0.13 g, 0.53 mmole) and methyl (triphenylphosphoranylidene)acetate (0.35 g, 1.05 mmole) in THF (3 mL) was heated at reflux to 5 hr, then was allowed to cool to RT. The reaction was poured into H₂O and the mixture was extracted with Et₂O. The organic extracts were dried over Na₂SO₄ and the solvent was removed under reduced pressure. Radial chromatography (20% EtOAc/hexane, silica gel, 6 mm plate) gave the title compound (0.12 g) as a mixture of olefin stereo- and regio-isomers. This was used in the next step without further purification: MS (ES) m/e 299.1 (M+H)⁺.

### d) Methyl 2-(2-phenoxy-4-methoxy)phenylbutanoate

A Parr hydrogenation vessel was charged with methyl 2-(2-phenoxy-4-methoxy)phenylbut-2-enoate (0.12 g, 0.39 mmole), 10% Pd/C (50 mg), and MeOH (50 mL), and the mixture was shaken under an atmosphere of hydrogen at 50 psi. After 18 hr, the catalyst was removed by filtration and the filtrate was concentrated under reduced pressure. Flash chromatography on silica gel (15% EtOAc/hexanes) gave the title compound (0.09 g) as a clear oil: MS (ES) m/e 300.9 (M+H)⁺.

### e) Methyl 2-(2-phenoxy-4-hydroxy)phenylbutanoate

A solution of methyl 2-(2-phenoxy-4-methoxy)phenylbutanoate (0.09 g, 0.30 mmole) in CH₂Cl₂ (2 mL) was added to BBr₃ (1.0M in CH₂Cl₂, 1.50 mL, 1.50 mmole) at 0° C. After 1 hr at 0° C., the reaction was quenched by dropwise addition of MeOH (2 mL). The solvent was removed under reduced pressure and the residue was azeotroped from MeOH (2×). A solution of saturated NaHCO₃ was added to the residue and the aqueous layer was extracted with EtOAc. The combined extracts were dried over Na₂SO₄ and concentrated to give the title compound (0.08 g) as a pale yellow oil. This material was used in the next step without further purification: ¹H NMR (300 MHz, CDCl₃) δ7.25 (m, 2 H), 7.05 (m, 2 H), 6.93 (d, J=7.6 Hz, 2 H), 6.54 (dd, J=8.2, 2.5 Hz, 1 H), 6.35 (d, J=2.5 Hz, 1 H), 5.45 (s, 1 H), 3.62 (s, 3 H), 2.59 (t, J=7.5 Hz, 2 H), 2.32 (t, J=7.5 Hz, 2 H), 1.90 (m,2H).

### Preparation 25

### Preparation of 2-(5,6,7,8-tetrahydro-1,8-naphthyridin-2-yl)-1-ethanol

### a) 2-Methyl-8-(tert-butoxycarbonyl)-5,6,7,8-tetrahydro-1,8-naphthyridine

A mixture of 2-methyl-1,8-naphthyridine (J. Chem. Soc. (C) 1966, 315; 5.13 g, 35.58 mmole), 10% Pd/C (1.14 g, 1.07 mmole), and absolute EtOH (70 mL) was deoxygenated through three evacuation/H₂ purge cycles, then was stirred briskly under a balloon of H₂. After 18.5 hr, the mixture was filtered through celite®, and the filter pad was washed sequentially with absolute EtOH and EtOAc. The filtrate was concentrated to dryness, and the residue was reconcentrated from EtOAc to leave an off-white solid (5.25 g).

A solution of the above material (5.25 g). di-tert-butyl dicarbonate (15.53 g, 71.16 mmole), and CH₂Cl₂ (10 mL) was concentrated on the rotavap to remove the solvent, and the oily residue was heated under N₂ in an oil bath set at 55-60° C. After 45 hr, the reaction was cooled to RT, and the residue was flash chromatographed on silica gel (40% EtOAc/hexanes). The title compound (4.90 g, 55%) was obtained as a light yellow solid: ¹H NMR (300 MHz, CDCl₃) δ7.27 (d, J=7.6 Hz, 1 H), 6.81 (d, J=7.6 Hz, 1 H), 3.69-3.79 (m, 2 H), 2.65-2.75 (m, 2 H), 2.48 (s, 3 H), 1.83-1.98 (m, 2 H), 1.52 (s, 9 H); MS (ES) m/e 249 (M+H)⁺.

### b) Ethyl [8-(tert-butoxycarbonyl)-5,6,7,8-tetrahydro-1,8-naphthyridin-2-yl]acetate

To a solution of diisopropylamine (7.24 mL, 55.3 mmole) in dry THF (50 mL) was added n-BuLi (2.5 M in hexanes, 22 mL, 55.3 mmole) dropwise at 0° C. After 15 min, this solution was added dropwise to a solution of 2-methyl-8-(tert-butoxycarbonyl)-5,6,7,8-tetrahydro-1,8-naphthyridine (4.9 g, 19.7 mmole) and diethylcarbonate (8.86 mL, 73.0 mmole) in dry THF (50 mL) at −78° C. After 30 min, the mixture was quenched with saturated NH₄Cl (100 mL), warmed to RT, and extracted with EtOAc (3×200 mL). The combined organic extracts were dried over MgSO₄, filtered, and concentrated under reduced pressure. The residue was chromatographed on silica gel (40% EtOAc/hexanes) to give the title compound (5.72 g, 91%) as a light yellow oil: MS (ES) m/e 321 (M+H)⁺.

### c) 2-(5,6,7,8-Tetrahydro-1,8-naphthyridin-2-yl)-1-ethanol

To a solution of ethyl [8-(tert-butoxycarbonyl)-5,6,7,8-tetrahydro-1,8-naphthyridin-2-yl]acetate (5.72 g, 17.85 mmole) in dry THF (80 mL) at RT was added LiBH₄ (2.0 M in THF, 10.7 mL, 21.42 mmole), and the resulting mixture was heated to reflux. After 18 hr, the mixture was cooled to 0° C. and carefully quenched with H₂O (100 mL). After 10 min, the mixture was extracted with EtOAc (3×100 mL). The combined organic extracts were dried over MgSO₄, filtered, and concentrated under reduced pressure.

The above residue (4.9 g) was dissolved in CH₂Cl₂ (10 mL). To this was added 4 N HCl in dioxane (20 mL) all at once at RT. After 4, the mixture was concentrated under reduced pressure. The residue was taken up in a 1:1 mixture of 1.0 N NaOH and saturated NaCl (100 mL) and extracted with CH₂Cl₂ (3×100 mL). The combined organic extracts were dried over MgSO₄, filtered, and concentrated under reduced pressure. The residue was chromatographed on silica gel (10% MeOH in 1:1 EtOAc/CHCl₃) to give the title compound (2.09 g, 66%) as a yellow solid: MS (ES) m/e 179 (M+H)⁺.

### Preparation 26

### HPLC separation of the enantiomers of methyl (±)-4-(4hydroxyphenyl)-3-(thiazol-2-yl)butanoate

### a) Methyl (S)-(−)-4-(4-hydroxyphenyl)-3-(thiazol-2-yl)butanoate and methyl (R)-(+)-4-(4-hydroxyphenyl)-3-(thiazol-2-yl)butanoate

Methyl (±)-4-(4hydroxyphenyl)-3-(thiazol-2-yl)butanoate was resolved into its enantiomers using the following conditions: Daicel Chiralcel OJ® column (21.2×250 mm), 20% ethanol in hexane mobile phase, 12 mL/min flow rate, uv detection at 320 nm, 25 mg injection; t_{R} for methyl (S)-(−)-4-(4-hydroxyphenyl)-3-(thiazol-2-yl)butanoate=14.5 min; t_{R} for methyl (R)-(+)-4-(4-hydroxyphenyl)-3-(thiazol-2-yl)butanoate=17.2 min.

### Preparation 27

### HPLC separation of the enantiomers of ethyl (±)-4-(4-methoxyphenyl)-3-phenylbutanoate

### a) Ethyl (−)-4-(4-methoxyphenyl)-3-phenylbutanoate and ethyl (+)-4-(4-methoxyphenyl)-3-phenylbutanoate

Ethyl (±)-4-(4-methoxyphenyl)-3-phenylbutanoate was resolved into its enantiomers using the following conditions: Daicel Chiralcel OJ® column (21.2×250 mm), 15% ethanol in hexane mobile phase, 15 mL/min flow rate, uv detection at 254 nm, 100 mg injection; t_{R} for ethyl (−)-4-(4-methoxyphenyl)-3-phenylbutanoate=9.0 min; t_{R} for ethyl (+)-4-(4-methoxyphenyl)-3-phenylbutanoate=12.2 min.

### Preparation 28

### Preparation of methyl (±)-3-(furan-2-yl)-4-(4-hydroxyphenyl)butanoate

### a) Methyl 3-(furan-2-yl)acrylate

H₂SO₄ (0.5 mL, 9.39 mmole) was added to a solution of 3-(2-furanyl)acrylic acid (5.0 g, 36.2 mmole) in MeOH (30 mL). The reaction was heated at reflux for 22 hr, then was concentrated on the rotavap. The residue was diluted with H₂O (100 mL) and extracted with ether (2×70 mL). The organic layers were combined and washed sequentially with saturated NaHCO₃ (30 mL) and H₂O (30 mL). Drying (Na₂SO₄) and concentration on the rotavap gave the title compound (4.86 g, 88%) as a light brown oil: TLC R_{f} (10% EtOAc/hexanes) 0.50; MS (ES) m/e 479.0 (3M+Na)⁺.

### b) Methyl (±)-3-(furan-2-yl)-4-(4-methoxyphenyl)butanoate

TMEDA (2.18 mL, 14.47 mmole) was added slowly to a mixture of CuI (2.51 g, 13.16 mmole) in THF (35 mL) at RT under argon. After 10 min at RT, the reaction mixture was cooled to −78° C., and a solution of 4-methoxybenzylmagnesium chloride in THF (0.5 M, 26.32 mL, 13.16 mmole) was added slowly. The reaction was stirred for 15 min, then a solution of TMSCl (4.17 mL, 32.89 mmole) and methyl 3-(furan-2-yl)acrylate (1.0 g, 6.58 mmole) in THF (20 mL) was injected, and the temperature was allowed to rise to −30° C. After 18 hr, the reaction was quenched with saturated NH₄Cl/NH₄OH (30 mL), and stirring was continued to an ambient temperature. H₂O (20 mL) was added, and the mixture was extracted with ether (2×70 mL). The combined organic layers were washed with H₂O (2×50 mL) an dried (Na₂SO₄). Concentration and silica gel chromatography (8% EtOAc/Hexanes) gave the title compound (0.85 g, 93%) as a clear oil: TLC R_{f} (8% EtOAc/Hexanes) 0.38; MS (ES) m/e 297 (M+Na)⁺.

### c) Methyl (±)-3-(furan-2-yl)-4-(4-hydroxyphenyl)butanoate

A solution of methyl (±)-3-(furan-2-yl)-4-(4-methoxyphenyl)butanoate (0.82 g, 2.99 mmole) in CH₂Cl₂ (10 mL) was added dropwise to a solution of BBr₃ in CH₂Cl₂ (1.0 M, 11.97 mL, 11.97 mmole) at 0° C. under argon. After 30 min, the reaction was quenched with MeOH (5 mL). The solution was stirred for 10 min then was concentrated on the rotavap. The residue was partitioned between EtOAc (50 mL) and 5% NaHCO₃ (30 mL). The layers were separated and the organic layer was washed with H₂O (20 mL) and dried (Na₂SO₄). Concentration and silica gel chromatography (40% EtOAc/Hexanes) gave the title compound (0.12 g, 15%) as a light yellow greenish residue: TLC R_{f} (50% EtOAc/hexanes) 0.36; MS (ES) m/e 542.8 (2M+Na)⁺.

### Preparation 29

### Preparation of (±)-3-[1-(dimethylaminosulfonyl)imidazol-2-yl]-4-(4-hydroxyphenyl)butanoate

### a) 1-(Dimethylaminosulfonyl)imidazole

To a solution of imidazole (1.63 g, 24 mmole) in CH₂Cl₂ (100 mL) was added Et₃N (3.35 mL, 24 mmole), followed by dimethylaminosulfonyl chloride (2.15 mL, 20 mmole) at RT. After 24 hr the mixture was concentrated. The residue was taken up in EtOAc (200 mL) and filtered through a pad of silica gel. The filtrate was concentrated to give the title compound (2.89 g, 82%) as a white solid: MS (ES) m/e 176 (M+H)⁺.

### b) 2-(4-Benzyloxyphenyl)-1-[1-(dimethylaminosulfonyl)imidazol-2-yl]ethanone

According to the procedure of Preparation 16 (a), except substituting 1-(dimethylaminosulfonyl)imidazole (410 mg, 2.34 mmole) for the 2-bromothiazole, the title compound (364 mg, 47%) was prepared as a white solid following silica gel chromatography (35% EtOAc/hexanes): MS (ES) m/e 400 (M+H)⁺.

### c) Ethyl (±)4-(4-benzyloxyphenyl)-3-[1-(dimethylaminosulfonyl)imidazol-2-yl]crotonate

According to the procedure of Preparation 16 (b), except substituting 2-(4-benzyloxyphenyl)-1-[1-(dimethylaminosulfonyl)imidazol-2-yl]ethanone (564 mg, 1.41 mmole) for the 2-[4-(benzyloxy)phenyl]-1-(thiazol-2-yl)ethanone, the title compound (589 mg of a mixture of olefin isomers, 89%) was prepared as an orange oil following silica gel chromatography (35% EtOAc/hexanes): MS (ES) m/e 470 (M+H)⁺.

### d) Ethyl (±)-3-[1-(dimethylaminosulfonyl)imidazol-2-yl]-4(hydroxyphenyl)butanoate

According to the procedure of Preparation 16 (c), except substituting ethyl (±)-4-(4-benzyloxyphenyl)-3-[1-(dimethylaminosulfonyl)imidazol-2-yl]crotonate (589 mg, 1.25 mmole) for the ethyl (±)-4-[4-(benzyloxy)phenyl]-3-(thiazol-2-yl)crotonate, the title compound (436 mg, 91%) was prepared as a white solid: MS (ES) m/e 382 (M+H)⁺.

### Preparation 30

### Preparation of ethyl (±)-3-(benzothiazol-2-yl)-4-(4-hydroxyphenyl)butanoate

### a) 1-(Benzothiazol-2-yl)-2-(4-benzyloxyphenyl)ethanone

According to the procedure of Preparation 16 (a), except substituting benzothiazole (0.26 mL, 2.34 mmole) for the 2-bromothiazole, the title compound (570 mg, 81%) was prepared as a pale yellow solid following trituration with hexanes: MS (ES) m/e 360 (M+H)⁺.

### b) Ethyl (±)-3-(benzothiazol-2-yl)-4-(4-benzyloxyphenyl)crotonate

According to the procedure of Preparation 16 (b), except substituting 1-(benzothiazol-2-yl)-2-(4-benzyloxyphenyl)ethanone (570 mg, 1.59 mmole) for the 2-[4-(benzyloxy)phenyl]-1-(thiazol-2-yl)ethanone, the title compound was prepared as a mixture of olefin isomers. The crude product was used without further purification.

### c) Ethyl (±)-3-(benzothiazol-2-yl)-4-(4-benzyloxyphenyl)butanoate

Ethyl (±)-3-(benzothiazol-2-yl)-4-(4-benzyloxyphenyl)crotonate (1.59 mmole, crude) was hydrogenated (50 psi H₂) using 10% Pd/C (1.00 g) in 1:1 EtOH/EtOAc (20 mL) for 5 hr. The mixture was filtered through a pad of celite®, and the filtrate was concentrated. The crude residue was used without further purification.

### d) Ethyl (±)-3-(benzothiazol-2-yl)-4-(4hydroxyphenyl)butanoate

To a solution of ethyl (±)-3-(benzothiazol-2-yl)-4-(4-benzyloxyphenyl)butanoate (1.59 mmole, crude) in EtSH (1.95 mL) at RT was added BF₃.OEt₂ (1.95 mL). After 48 hr, additional BF₃.OEt₂ (1.95 mL) was added. After another 18 hr, the mixture was cooled to 0° C. and carefully quenched with saturated NaHCO₃. The resulting mixture was extracted with CH₂Cl₂ (3×25 mL). The combined organic layers were dried over MgSO₄ and concentrated. The residue was chromatographed on silica gel (30% EtOAc/hexanes) to give the title compound (391 mg, 72% over 3 steps) as a foam: MS (ES) m/e 342 (M+H)⁺.

### Preparation 31

### Preparation of ethyl (±)-3-(4-methylthiazol-2-yl)-4-(4-hydroxyphenyl)butanoate

### a) 2-(4-Benzyloxyphenyl)-1-(4-methylthiazol-2-yl)ethanone

According to the procedure of Preparation 16 (a), except substituting 4-methylthiazole (0.21 mL, 2.34 mmole) for the 2-bromothiazole, the title compound (303 mg, 48%) was prepared as a pale yellow solid following silica gel chromatography (15% EtOAc/hexanes): MS (ES) m/e 324 (M+H)⁺.

### b) Ethyl (±)-3-(4-methylthiazol-2-yl)-4-(4-benzyloxyphenyl)crotonate

According to the procedure of Preparation 16 (b), except substituting 2-(4-benzyloxyphenyl)-1-(4-methylthiazol-2-yl)ethanone (300 mg, 0.93 mmole) for the 2-[4-(benzyloxy)phenyl]-1-(thiazol-2-yl)ethanone, the title compound was prepared as a mixture of olefin isomers. The crude product was used without further purification.

### c) Ethyl (±)-3-(4-methylthiazol-2-yl)-4-(4-benzyloxyphenyl)butanoate

Ethyl (±)-3-(4-methylthiazol-2-yl)-4-(4-benzyloxyphenyl)crotonate (0.93 mmole, crude) was dissolved in MeOH (10 mL), and magnesium turnings (113 mg, 4.65 mmole) were added at RT. After 18 hr the mixture was poured into 10% HCl (75 mL) and extracted with CH₂Cl₂ (3×50 mL). The combined organic layers were dried over MgSO₄ and concentrated. The residue was used in the next step without purification.

### d) Ethyl (±)-3-(4-methylthiazol-2-yl)4-(4-hydroxyphenyl)butanoate

To a solution of ethyl (±)-3-(4-methylthiazol-2-yl)-4-(4-benzyloxyphenyl)butanoate (0.93 mmole, crude) in EtSH (10 mL) was added BF₃.OEt₂ (2.29 mL) at RT. After 24 hr, more BF₃.OEt₂ (1.00 mL) was added. After 72 hr the mixture was cooled to 0° C. and carefully quenched with saturated NaHCO₃. The resulting mixture was extracted with CH₂Cl₂ (3×25 mL). The combined organic layers were dried over MgSO₄ and concentrated. The residue was chromatographed on silica gel (30% EtOAc/hexanes) to give the title compound (216 mg, 80% over 3 steps) as a white solid: MS (ES) m/e 292 (M+H)⁺.

### Preparation 32

### Preparation of methyl (±)-4-(4-hydroxyphenyl)-3-[4-(benzyloxycarbonyl)1,3-oxazol-2-yl]butanoate

### a) 4-Bromo-1-(triisopropylsilyloxy)benzene

To a solution of 4-bromophenol (2.00 g, 11.56 mmole) in dry DMF (20 mL) at RT was added imidazole (1.57 g, 23.12 mmole), followed by triisopropylsilyl chloride (3.71 mL, 17.34 mmole). After 4 hr the mixture was diluted with H₂O (50 mL) and extracted with hexanes (3×75 mL). The combined organic layers were dried over MgSO₄ and concentrated to give the title compound (4.00 g, 100%) as a clear oil which was used without purification: ¹H NMR (300 MHz, CDCl₃) δ7.29 (d, J=6 Hz, 2 H), 6.71 (d, J=6 Hz, 2 H), 1.22 (m, 3 H), 1.09 (m, 18 H).

### b) Methyl (±)-3-carboxy-4-[4-(triisopropylsilyloxy)pheny]butanoate

According to the procedure of Preparation 15 (b), except substituting 4-bromo-1-(triisopropylsilyloxy)benzene (2.19 g, 6.66 mmole) for the 4-bromoanisole, the title compound (2.24 g, 85% over 2 steps) was prepared as a clear oil: ¹H NMR (300 MHz, CDCl₃) δ7.01 (d, J=6 Hz, 2 H), 6.80 (d, J=6 Hz, 2 H), 3.62 (s, 3 H), 3.05 (m, 2 H), 2.65 (m, 1 H), 2.40 (m, 2 H), 1.21 (m, 3H), 1.09 (m, 18H).

### c) (±)-N-[2-[4-(Triisopropylsilyloxy)benzyl]-3-(carbomethoxy)propionyl]serine benzyl ester

To a solution of methyl (±)-3-carboxy-4-[4-(triisopropylsilyloxy)phenyl]butanoate (1.00 g, 2.53 mmole) in dry DMF (10 mL) at RT was added serine benzyl ester hydrochloride (704 mg, 3.04 mmole), HOBt (411 mg, 3.04 mmole), Et₃N (1.06 mL, 7.60 mmole), and EDC (583 mg, 3.04 mmole). After 18 hr the mixture was concentrated. The residue was chromatographed on silica gel (80% EtOAc/hexanes) to give the title compound (834 mg, 58%) as a pale yellow oil: MS (ES) m/e 572 (M+H)⁺.

### d) Methyl (±)-3-[4-(benzyloxycarbonyl)-1,3-oxazolin-2-yl]-4-[4-(triisopropylsilyloxy)phenyl]butanoate

To a solution of (±)-N-[2-[4-(triisopropylsilyloxy)benzyl]-3-(carbomethoxy)propionyl]serine benzyl ester (834 m,, 1.46 mmole) in dry THF (10 mL) was added Burgess reagent (417 mg, 1.75 mmole), then the mixture was heated to reflux. After 2 hr the mixture was cooled to RT and concentrated. The residue was chromatographed on silica gel (35% EtOAc/hexanes) to give the title compound (633 mg. 78%) as a clear oil: MS (ES) m/e 554 (M+H)⁺.

### e) Methyl (±)-3-[4-(benzyloxycarbonyl)-1,3-oxazol-2-yl]-4-[4-(triisopropylsilyloxy)phenyl]butanoate

To a solution of methyl (±)-3-[4-(benzyloxycarbonyl)-1,3-oxazolin-2-yl]-4-[4-(triisopropylsilyloxy)phenyl]butanoate (633 mg, 1.14 mmole) in CH₂Cl₂ (6 mL) at 0° C. was added DBU (0.19 mL, 1.25 mmole), followed by bromotrichloromethane (0.12 mL, 1.25 mmole). The mixture was allowed to warm to RT as the bath warmed. After 18 hr the mixture was concentrated. The residue was chromatographed on silica gel (20% EtOAc/hexanes) to give the title compound (427 mg, 68%) as a clear oil: MS (ES) m/e 552 (M+H)⁺.

### f) Methyl (±)-3-[4-(benzyloxycarbonyl)-1,3-oxazol-2-yl]-4-(4-hydroxyphenyl)butanoate

To a solution of methyl (±)-3-[4-(benzyloxycarbonyl)-1,3-oxazol-2-yl]-4-[4-(triisopropylsilyloxy)phenyl]butanoate (427 mg, 0.77 mmole) in dry THF (5 mL) at 0° C. was added a solution of TBAF in THF (1.0 M, 1.16 mL, 1.16 mmole). After 2 hr the mixture was diluted with saturated NH₄Cl (10 mL) and extracted with CH₂Cl₂ (3×15 mL). The combined organic layers were dried over MgSO₄ and concentrated. The residue was chromatographed on silica gel (40% EtOAc/hexanes) to cive the title compound (268 mg, 88%) as an off-white foam: MS (ES) m/e 396 (M+H)⁺.

### Preparation 33

### Preparation of methyl (±)-3-[4-carboxy-1,3-oxazol-2-yl]-4-[4-[2-[6-(methylamino)pyridin-2-yl]-1-ethoxy]phenyl]butanoate

### a) Methyl (±)-3-[4-(benzyloxycarbonyl)-1,3-oxazol-2-yl]-4-[4-[2-[6-(methylamino)pyridin-2-yl]-1-ethoxy]phenyl]butanoate

Diisopropyl azodicarboxylate (0.27 mL, 1.36 mmole) was added to a solution of methyl (±)-3-[4-(benzyloxycarbonyl)-1,3-oxazol-2-yl]-4-(4-hydroxyphenyl)butanoate (268 mg, 0.68 mmole), 2-[(6-methylamino)pyridin-2-yl)]ethanol (207 mg, 1.36 mmole), and triphenylphosphine (357 mg, 1.36 mmole) in anhydrous THF (4 mL) at 0° C. The mixture was allowed to warm to RT as the bath warmed. After 18 hr the mixture was concentrated and the residue was chromatographed on silica gel (50% EtOAc/toluene) to give the title compound (284 mg, 79%) as a clear oil: MS (ES) m/e 530 (M+H)⁺.

### b) Methyl (±)-3-[4-carboxy-1,3-oxazol-2-yl]-4-[4-[2-[6-(methylamino)pyridin-2-yl]-1-ethoxy]phenyl]butanoate

A mixture of methyl (±)-3-[4-(benzyloxycarbonyl)-1,3-oxazol-2-yl]-4-[4-[2-[6-(methylamino)pyridin-2-yl]-1-ethoxy]phenyl]butanoate (234 mg, 0.44 mmole) and 10% Pd/C (100 mg) in EtOH (5 mL) was deoxygenated (3×vacuum/N₂), then was stirred briskly under H₂, (balloon pressure). After 4 hr the mixture was filtered through a pad of celite® and concentrated to give the title compound (165 mg, 85%) as a white foam: MS (ES) m/e 440 (M+H)⁺.

### Preparation 34

### Preparation of methyl (±)-3-(4-hydroxybenzyl)pent-4-ynoate

### a) Methyl (±)-3-formyl-4-(4-methoxyphenyl)butanoate

To a solution of methyl (±)-4-(4-methoxyphenyl)-3-carboxybutanoate (prepared as described in Preparation 15, 0.45 g, 1.80 mmole) in CH₂Cl₂ (10 mL) was added oxalyl chloride (0.24 mL, 2.75 mmole) and DMF (1 drop). After 1.5 hr, the solvent was removed under reduced pressure and the residue was azeotroped from toluene (2×). The crude acid chloride was dissolved in acetone (2 mL) and the solution was added dropwise to a rapidly stirring suspension of (Ph₃P)₂CuBH₄ (1.14 g, 1.89 mmole) and Ph₃P (0.99 g, 3.78 mmole) in acetone (4 mL). After 1 h at RT, the reaction mixture was filtered through celite®, and the filter pad was further rinsed with EtOAc. The combined organic filtrates were concentrated to give a yellow residue. Radial chromatography on silica gel (6 mm plate, 20% EtOAc/hexane) gave the title compound (0.25 g) as a clear oil: ¹H NMR (300 MHz, CDCl₃) δ9.79 (s, 1 H), 7.11 (d, J=8.6 Hz, 2 H), 6.84 (d, J=8.6 Hz, 2 H), 3.79 (s, 3 H), 3.65 (s, 3 H), 3.10 (m, 2 H), 2.70 (m, 2 H), 2.38 (dd, J=16.8, 5.1 Hz, 1 H).

### b) Methyl (±)-3-(4-methoxybenzyl)pent-4-ynoate

To a solution of methyl (±)-3-formyl-4-(4-methoxyphenyl)butanoate (0.14 g, 0.61 mmole) in dry MeOH (5 mL) was added K₂CO₃ (0.17 g, 1.21 mmole), followed by dropwise addition of a solution of dimethyl-1-diazo-2-oxopropylphosphonate (0.13 g, 0.67 mmole) in MeOH (5 mL). After 18 hr at RT, the reaction was poured into sat. NaHCO₃ and extracted with Et₂O. The combined organic extracts were washed with brine and dried over MgSO₄. The solvent was removed under reduced pressure to give a clear oil. Radial chromatography on silica gel (2 mm plate, 20% EtOAc/hexanes) gave the title compound (0.06 g) as a clear oil: ¹H NMR (300 MHz, CDCl₁₃) δ7.23 (d, J=8.4 Hz, 2 H), 6.92 (d, J=8.4 Hz, 2 H), 3.87 (s, 3 H), 3.77 (s, 3 H), 3.20 (m, 1 H), 2.85 (m, 2 H), 2.56 (d, 6.7 Hz, 2 H), 2.17 (d, J=2.0 Hz, 1 H).

### c) Methyl (±)-3-(4-hydroxybenzyl)pent-4-ynoate

To a solution of BBr₃ in CH₂Cl₂ (1.0 M, 0.85 mL, 0.85 mmole) at 0° C. was added a solution of methyl (±)-3-(4-methoxybenzyl)pent-4-ynoate (66 mg, 0.28 mmole) in CH₂Cl₂ (0.60 mL). After 3 hr at 0° C., the reaction was quenched by careful addition of MeOH (1 mL). The solvent was removed under reduced pressure and the residue was azeotroped from MeOH (2×). Sat.NaHCO₃ was added to the residue and the aqueous layer was extracted with EtOAc. The combined organic extracts were washed with brine and dried over Na₂SO₄. The solvent was removed under reduced pressure to give a clear film. Radial chromatography on silica gel (2 mm plate, 20% EtOAc/hexanes) gave the title compound (25 mg) as a clear film: ¹H NMR (300 MHz, CDCl₃) δ7.15 (d, J=8.5 Hz, 2 H), 6.83 (d, J=8.5 Hz, 2 H), 3.79 (s, 3 H), 3.69 (s, 3 H), 3.10 (m, 1 H), 2.75 (m, 2 H), 2.45 (m, 2 H), 2.11 (d, J=2.2 Hz, 1 H).

### Preparation 35

### Preparation of methyl (±)-4-(4-hydroxyphenyl)-3-(phenylethyl)butanoate

### a) (±)-2-(4-Methoxybenzyl)-4-phenylbutanoic acid

A reaction flask was charged with diisopropylamine (1.0 mmole, 7.5 mmole), NaH (60% in mineral oil, 0.33 g, 8.5 mmole), and THF (40 mmole). To the stirred mixture was added a solution of phenylbutyric acid (1.23 g, 7.5 mmole) in THF (10 mmole) over 5 minutes. The hydrogen evolution was completed by heating the mixture to reflux for 10 minutes. The reaction was cooled to 10° C., and a solution of n-BuLi (2.5 M in hexanes, 3.0 mmole, 7.5 mmole) was added. After 15 minutes at that temperature the mixture was heated to 30° C. for 15 min. The turbid solution was cooled to 0° C. and 4-methoxybenzyl chloride (1.2 g, 7.5 mmole) was added over 10 minutes. After 20 minutes at that temperature the mixture was stirred at RT overnight. The reaction was kept at or below 15° C. while H₂O (50 mL) was added. The mixture was partly concentrated in vacuum, diluted with water, and extracted with ether (2×50 mL). The aqueous layer was acidified with 6 N HCl to Congo red and extracted with Et₂O (3×30 mL). The combined extracts were dried over anhydrous MgSO₄, filtered and concentrated to give the title compound (1.6 g, 56%) as a yellow oil: TLC R_{f} (1% MeOH/CH₂Cl₂) 0.37.

### b) (±)-1-Diazo-4-(4-methoxyphenyl)-3-(2-phenylethyl)-2-butanone

A solution of (±)-2-(4-methoxybenzyl)-4-phenylbutanoic acid (1.5 g, 5.26 mmole) in CH₂Cl₂ (30 mL) was treated with oxalyl chloride (0.92 mL, 10.5 mmole). The reaction was stirred at RT overnight, then was concentrated in vacuum. The residue was dissolved in Et₂O, and Et₃N was added, followed by excess diazomethane (generated from 1-methyl-3 nitro-1-nitroguanidine and NaOH). The reaction was stirred at RT overnight then was concentrated in vacuum to afford the title compound (1.5 g 94%) as a yellow oil: MS (ES) m/e 309 (M+H)⁺.

### c) Methyl (±)-4-(4-methoxyphenyl)-3-(2-phenylethyl)butanoate

A solution of silver benzoate (0.9 g 3.9 mmole) in Et₃N (8 mL, 55.6 mL) was added to a solution of (±)-1-diazo-4-(4-methoxyphenyl)-3-(phenylethyl)-2-butanone (0.3 g, 0.97 mmole) in MeOH (20 mL) at RT. Gas evolution was observed, and the reaction mixture became black in color. After 30 min, the reaction was heated to reflux. After 1 hr at reflux, the reaction was cooled to RT and filtered through celite®, and the filtrate was concentrated in vacuum. The residue was adsorbed onto silica gel and was loaded onto a dry silica gel column. Flash chromatography (5% EtOAc/hexanes) gave the title compound (0.1 g, 57%) as a light yellow oil: TLC R_{f} (5% EtOAc/hexanes) 0.63.

### d) Methyl (±)-4-(4-hydroxyphenyl)-3-(phenylethyl)butanoate

Boron tribromide (1.0 M in CH₂Cl₂, 4.8 mL, 4.8 mmole) was added to a solution of methyl (±)-4-(4-methoxyphenyl)-3-(2-phenylethyl)butanoate (1.0 g, 3.21 mmole) in CH₂Cl₂ (10 mL) at 0° C. under argon. After 1 hr, the reaction was quenched with absolute MeOH and concentrated in vacuum. Reconcentration from toluene (several times) followed by drying in high vacuum gave the title compound (0.7 g, 73% ) as an oil: TLC R_{f} (15% EtOAc/hexanes) 0.26.

### Preparation 36

### Preparation of methyl (±)-4-(4-hydroxyphenyl)-3-benzylbutanoate

### a) (±)-2-(4-Methoxybenzyl)-3-phenylpropionic acid

According to the procedure of Preparation 35 (a), except substituting phenylpropionic acid for the phenylbutyric acid, the title compound (60%) was obtained as yellow oil: TLC R_{f} (1% MeOH/CH₂Cl₂) 0.38.

### b) (±)-1-Diazo-3-(4-methoxyphenyl)-3-(benzyl)-2-butanone

According to the procedure of Preparation 35 (b), except substituting (±)-2-(4-methoxybenzyl)-3-phenylpropionic acid for the (±)-2-(4-methoxybenzyl)-4-phenylbutanoic acid, title compound (100%) was obtained as yellow oil: MS (ES) m/e 289 (M+H)⁺.

### c) Methyl (±)-4-(4-methoxyphenyl)-3-benzylbutanoate

According to the procedure of Preparation 35 (c). except substituting (±)-1-diazo-3-(4-methoxyphenyl)-3-(benzyl)-2-butanone for the (±)-1-diazo-4-(4-methoxyphenyl)-3-(phenylethyl)-2-butanone, the title compound (80%) was prepared as a slightly yellow foam: TLC R_{f} (5% EtOAc/hexanes) 0.33.

### d) Methyl (±)-4-(4-hydroxyphenyl)-3-benzylbutanoate

According to the procedure of Preparation 35 (d), except substituting methyl (±)-4-(4-methoxyphenyl)-3-benzylbutanoate for the methyl (±)-4-(4-methoxyphenyl)-3-(2-phenylethyl)butanoate, the title compound (24%) was prepared: TLC R_{f} (20% EtOAc/hexanes) 0.33.

### Preparation 37

### Preparation of methyl (±)-4-(4-hydroxyphenyl)-3-cyclopropylbutanoate

### a) (±)-2-(4-Methoxybenzyl)-2-cyclopropyl acetic acid

According to the procedure of Preparation 35 (a), except substituting cyclopropylacetic acid for the phenylbutyric acid, the title compound (60%) was obtained as yellow oil: TLC R_{f} (10% MeOH/CH₂Cl₂) 0.42.

### b) (±)-1-Diazo-3-(4-methoxyphenyl)-3-cyclopropyl-2-butanone

According to the procedure of Preparation 35 (b), except substituting (±)-2-(4-methoxybenzyl)-2-cyclopropyl acetic acid for the (±)-2-(4-methoxybenzyl)-4-phenylbutanoic acid, title compound (100%) was prepared as a yellow oil: MS (ES) m/e 245 (M+H)⁺.

### c) Methyl (±)-4-(4-methoxyphenyl)-3-cyclopropylbutanoate

According to the procedure of Preparation 35 (c), except substituting (±)-1-diazo-3-(4-methoxyphenyl)-3-cyclopropyl-2-butanone for the (±)-1-diazo-4-(4-methoxyphenyl)-3-(phenylethyl)-2-butanone, the title compound (60%) was prepared as a slightly yellow film: TLC R_{f} (10% EtOAc/hexanes) 0.21.

### d) Methyl (±)-4-(4-hydroxyphenyl)-3-cyclopropylbutanoate

According to the procedure of Preparation 35 (d), except substituting methyl (±)-4-(4-methoxyphenyl)-3-cyclopropylbutanoate for the methyl (±)-4-(4-methoxyphenyl)-3-(2-phenylethyl)butanoate, the title compound (20%) was prepared as a slightly yellow film: TLC R_{f} (10% EtOAc/hexanes) 0.11.

### Preparation 38

### Preparation of ethyl 4-(4-hydroxyphenyl)-3-methyl-3-butenoate

### a) Ethyl 4-(4-methoxyphenyl)-3-methyl-3-butenoate

To a suspension of NaH (60% in mineral oil, 2.1 g, 54 mmole) in toluene (40 mL) was added triethyl phosphonoacetate (11.1 g, 49.4 mmole) in toluene (50 mL). The reaction was stirred at RT for 20 min, then a solution of 4-methoxyphenylacetone (7.4 g, 44.9 mmole) in toluene (40 mL) was added dropwise. The reaction was heated at reflux for 5 hr, then was concentrated. Flash chromatography on silica gel (5% EtOAc/hexanes) gave the title compound (1.0 g) as a colorless oil: TLC Rf (5% EtOAc/hexanes) 0.23.

### b) Ethyl 4-(4hydroxyphenyl)-3-methyl-3-butenoate

According to the procedure of Preparation 35 (d), except substituting ethyl 4-(4-methoxyphenyl)-3-methyl-3-butenoate for the methyl (±)-4-(4-methoxyphenyl)-3-(2-phenylethyl)butanoate, the title compound (34%) was prepared as a colorless oil: TLC R_{f} (10% EtOAc/hexanes) 0.13.

The following compounds illustrate methods for preparing the biologically active compounds of this invention from intermediate compounds such as described in the foregoing Preparations.

### EXAMPLE 1

### Preparation of (±)-3-phenyl-4-[4-[3-(pyridin-2-yl)amino-1-propyloxy]phenyl]butanoic acid

### a) Ethyl (±)-3-phenyl-4-[4-[3-(1-oxopyridin-2-yl)amino-1-propyloxy]phenyl]butanoate

Diisopropyl azodicarboxylate (0.44 mL, 2.25 mmole) was added over 45 sec to a solution of ethyl (±)-4-(4-hydroxyphenyl)-3-phenylbutanoate (426.5 mg, 1.5 mmole), 2-[(3-hydroxy-1-propyl)amino]pyridine-N-oxide (378.5 mg, 2.25 mmole), and triphenylphosphine (590.2 mg, 2.25 mmole) in anhydrous DMF (22.5 mL) at 0° C. under argon. The yellow solution was kept at 0° C. for 10 min, then was warmed to RT. After 23 hr, the reaction was concentrated and the residue was reconcentrated from xylenes (2×). Silica gel chromatography (gradient: EtOAc, then 5% MeOH/CHCl₃) gave the title compound (445.7 mg, 68%) as a yellow oil: TLC R_{f} (5% MeOH/CHCl₃) 0.41; ¹H NMR (250 MHz, CDCl₃) δ8.11 (dd, J=6.5, 1.3 Hz, 1 H), 7.05-7.35 (m, 5 H), 6.85-7.05 (m, 1 H), 6.94 (d, J=8.6 Hz, 2 H), 6.76 (d, J=8.6 Hz, 2 H), 6.62 (dd, J=8.5, 1.5 Hz, 1 H), 6.48-6.59 (m, 1 H), 3.90-4.10 (m, 4 H), 3.50 (q, J=6.5 Hz, 2 H), 3.25-3.45 (m, 1 H), 2.85 (d, J=7.4 Hz, 2 H), 2.50-2.72 (m, 2 H), 2.05-2.22 (m, 2 H), 1.11 (t, J=7.1 Hz, 3 H); MS (ES) m/e 435.1 (M+H)⁺.

### b) Ethyl (±)-3-phenyl-4-[4-[3-(pyridin-2-yl)amino-1-propyloxy]phenyl]butanoate

A mixture of ethyl (±)-3-phenyl-4-[4-[3-(1-oxopyridin-2-yl)amino-1-propyloxy]phenyl]butanoate (445.7 mg, 1.03 mmole), cyclohexene (1 mL, 10 mmole), 10% Pd/C (110 mg, 0.10 mmole), and isopropanol (10 mL) was heated at reflux under argon. After 3 hr, more Pd/C (110 mg) was added. The mixture was heated at reflux for another 20.5 hr, then was hot-filtered through celite®. The filter pad was washed with hot 1:1 MeOH/CHCl3, and the combined filtrates were concentrated. The residue was reconcentrated from toluene, then was chromatographed on silica gel (5% MeOH/CHCl₃) to afford the title compound (332.5 mg, 77%) as a colorless oil: TLC R_{f} (5% MeOH/CHCl₃) 0.43; ¹H NMR (250 MHz, CDCl₃) δ8.02-8.12 (m, 1 H), 7.32-7.45 (m, 1 H), 7.06-7.32 (m, 5 H), 6.94 (d, J=8.6 Hz, 2 H), 6.75 (d, J=8.6 Hz, 2 H), 6.50-6.60 (m, 1 H), 6.39 (d, J=8.4 Hz, 1 H), 4.65-4.82 (m, 1 H), 3.88-4.10 (m, 4 H), 3.48 (q, J=6.4 Hz, 2 H), 3.28-3.45 (m, 1 H), 2.84 (d, J=7.4 Hz, 2 H), 2.50-2.62 (m, 2 H), 2.00-2.15 (m, 2 H), 1.10 (t, J=7.1 Hz, 3 H); MS (ES) m/e 419.1 (M+H)⁺.

### c) (±)-3-Phenyl-4-[4-[3-(pyridin-2-yl)amino-1-propyloxy]phenyl]butanoic acid

A mixture of ethyl (±)-3-phenyl-4-[4-[3-(pyridin-2-yl)amino-1-propyloxy]phenyl]butanoate (332.5 mg, 0.79 mmole), 1.0 N LiOH (1.2 mL, 1.2 mmole). THF (4 mL), and H₂O (2.8 mL) was stirred at RT for 4 hr, then was warmed in an oil bath set at 45-50° C. After 17.5 hr, the resulting homogeneous, nearly colorless solution was cooled to RT and extracted with Et₂O (2×8 mL). The Et₂O layers were discarded. The aqueous layer was stirred with gentle warming under vacuum to remove residual organic solvents, then was filtered. The resulting aqueous solution was stirred at RT while the pH was slowly and carefully adjusted to 5.5-6.0 with 1.0 N HCl. The mixture was stirred for 0.5 hr, then the solid was collected by suction filtration and washed with plenty of H₂O. Drying in high vacuum at 60° C. gave the title compound (232.3 mg, 74%) as a glassy solid: HPLC (Hamilton PRP-1®, 35% CH₃CN/H₂O containing 0.1% TFA) K′=2.4; ¹H NMR (400 MHz, CD₃OD) δ7.75-7.95 (m, 1 H), 7.48 (app t, 1 H), 7.07-7.27 (m, 5 H), 6.90 (d, J=8.5 Hz, 2 H), 6.72 (d, J=8.5 Hz, 2 H), 6.50-6.70 (m, 2 H), 4.01 (t, J=6.0 Hz, 2 H), 3.44 (t, J=6.7 Hz, 2 H), 3.20-3.40 (m, 1 H, obscured by residual solvent signal), 2.87 (dd, J=13.6, 6.6 Hz, 1 H), 2.79 (dd, J=13.6, 8.1 Hz, 1 H), 2.48-2.70 (m, 2 H), 1.98-2.11 (m, 2 H); MS (ES) m/e 391.0 (M+H)⁺. Anal. Calcd for C₂₄H₂₆N₂O₃.0.33 H₂O: C, 72.72; H. 6.78; N, 7.07. Found: C, 72.68; H, 6.69; N, 6.96.

### EXAMPLE 2

### Preparation of (±)-3-phenyl-4-[4-[2-[6-(methylamino)pyridin-2-yl]-1-ethoxy]phenyl]butanoic acid

### a) Ethyl (±)-3-phenyl-4-[4-[2-[6-(methylamino)pyridin-2-yl]-1-ethoxy]pheny]butanoate

Diisopropyl azodicarboxylate (0.44 mL, 2.25 mmole) was added over 2 min to a solution of ethyl (±)-4-(4-hydroxyphenyl)-3-phenylbutanoate (427 mg, 1.5 mmole), 6-(methylamino)-2-pyridylethanol (343 mg, 2.25 mmole), and triphenylphosphine (590 mg, 2.25 mmole) in anhydrous THF (22.5 mL) at 0° C. under N₂. The yellow solution was kept at 0° C. for 10 min, then was warmed to RT. After 24 hr, the reaction was concentrated and the residue was chromatographed on silica gel (4:1 Et₂O/hexanes). The title compound (479.5 mg, 76%) was obtained as a colorless oil: TLC R_{f} (4:1 Et₂O/hexanes) 0.50; ¹H NMR (250 MHz, CDCl₃) δ7.38 (app t, 1 H), 7.07-7.30 (m, 5 H), 6.93 (d, J=8.6 Hz, 2 H), 6.76 (d, J=8.6 Hz, 2 H), 6.54 (d, J=7.3 Hz, 1 H), 6.24 (d, J=8.3 Hz, 1 H), 4.42-4.58 (m, 1 H), 4.26 (t, J=7.0 Hz, 2 H), 3.98 (q, J=7.1 Hz, 2 H), 3.25-3.42 (m, 1 H), 3.05 (t, J=7.0 Hz, 2 H),2.89 (d, J=5.3 Hz, 3 H), 2.74-2.92 (m, 2 H), 2.50-2.72 (m, 2 H), 1.10 (t, J=7.1 Hz, 3 H); MS (ES) m/e 419.1 (M+H)⁺.

### b) (±)-3-Phenyl-4-[4-[2-[6-(methylamino)pyridin-2-yl]-1-ethoxy]phenyl]butanoic acid

1.0 N NaOH (1.15 mL, 1.15 mmole) was added dropwise to a cooled (15° C.) solution of ethyl (±)-3-phenyl-4-[4-[2-[6-(methylamino)pyridin-2-yl-1-ethoxy]phenyl]butanoate (479.5 mg, 1.15 mmole) in dioxane (4.6 mL). The resulting mixture was stirred at RT for 2.5 hr, then was warmed in an oil bath set at 40° C. After 24 hr, the reaction was cooled to RT and stirred for 3 days, then was diluted with H₂O (3.4 mL) and extracted with Et₂O (3×5 mL). The Et₂O layers were discarded. Since a solid precipitate separated from the aqueous layer, 1.0 N NaOH (1.0 mL), dioxane (5 mL), and Et₂O (10 mL) were added to afford a homogeneous solution. The pH was adjusted to 5.5-6.0 with 1.0 N HCl, and the organic solvents were removed on the rotavap. The aqueous solution was decanted away from the gummy precipitate, and the precipitate was dried thoroughly in vacuum. The residue was recrystallized from CH₃CN, and the solid was dried in vacuum at 60° C. for several days to afford the title compound (331.0 mg, 74%) as a white, crystalline solid: HPLC (Hamilton PRP-1®, 35% CH₃CN/H₂O containing 0.1% TFA) K′=2.9; ¹ H NMR (300 MHz, DMSO-d₆) δ7.05-7.40 (m, 6 H), 6.95 (d, J=8.4 Hz, 2 H), 6.76 (d, J=8.4 Hz, 2 H), 6.42 (d, J=7.1 Hz, 1 H), 6.30-6.50(m, 1 H), 6.26 (d, J=8.3 Hz, 1 H), 4.21 (t, J=6.7 Hz, 2 H), 3.12-3.30 (m, 1 H), 2.92 (t, J=6.7 Hz, 2 H), 2.60-2.90 (m, 2 H), 2.73 (d, J=4.8 Hz, 3 H), 2.40-2.60 (m, 2 H, partially obscured by residual solvent signal); MS (ES) m/e 391.2 (M+H)⁺. Anal. Calcd for C₂₄H₂₆N₂O₃: C, 73.82; H, 6.71; N, 7.17. Found: C, 73.43; H, 6.72; N, 7.40.

### EXAMPLE 3

### Preparation of (±)-3-phenyl-4-[4-[[2-(pyridin-2-yl)amino-1-ethylamino]carbonyl]phenyl]butanoic acid

### a) Ethyl (±)-3-phenyl-4-[4-[[2-(pyridin-2-yl)amino-1-ethylamino]carbonyl]phenyl]butanoate

To a suspension of ethyl (±)-(4-carboxyphenyl)-3-phenylbutanoate (312 mg, 1.0 mmoles), 2-[(2-amino-1-ethyl)amino]pyridine dihydrochloride (252 mg, 1.2 mmoles), and HOBt (162 mg, 1.2 mmoles) in CH₃CN (5 mL) was added (i-Pr)₂NEt (0.87 mL, 5.0 mmoles) then EDC (230 mg, 1.2 mmoles). After 18 hr the mixture was concentrated. The residue was chromatographed on silica gel (5% MeOH in 1:1 CHCl₃/EtOAc) to give the title compound (380 mg, 88%) as a brownish foam: MS (ES) m/e 432 (M+H)⁺.

### b) (±)-3-Phenyl-4-[4-[[2-(pyridin-2-yl)amino-1-ethylamino]carbonyl]phenyl]butanoic acid

To a solution of ethyl (±)-3-phenyl-4-[4-[[2-(pyridin-2-yl)amino-1-ethylamino]carbonyl]phenyl]butanoate (380 mg, 0.88 mmoles) in 1:1 THF/H₂O (5 mL) was added 1.0 N LiOH (1.3 mL, 1.3 mmoles). After 24 hr the mixture was concentrated to remove the THF. The resulting aqueous solution was cooled to 0° C. and acidified to pH 6 using 10% HCl. The precipitate was collected by filtration and dried in vacuo to give the title compound (213 mg, 60%) as a white solid: MS (ES) m/e 404 (M+H)⁺. Anal. Calcd for C₂₄H₂₅N₃O₃.0.25 H₂O: C, 70.66; H, 6.30; N, 10.30. Found: C, 70.92; H, 6.44; N, 10.14.

### EXAMPLE 4

### Preparation of (±)-3-phenyl-3-[4-[4-(pyridin-2-yl)amino-1-butyl]phenylamino]propanoic acid

### a) 1-Bromo-4-(4-nitrophenyl)butane

To a solution of 4-(4-nitrophenyl)-1-butanol (1.0 g, 5.12 mmoles) in dry THF (10 mL) was added PPh₃ (1.61 g, 6.14 mmoles) and CBr₄ (2.04 g, 6.14 mmoles). After 4 hr the mixture was concentrated. The residue was chromatographed on silica gel (10% EtOAc/hexanes) to afford the title compound (1.22 g, 92%) as a pale yellow oil: ¹H NMR (300 MHz, CDCl₃) ?8.18 (d, J=6.5 Hz, 2 H), 7.36 (d, J=6.5 Hz, 2 H), 3.48 (t, 2H), 2.80 (t, 2 H), 1.9 (m, 4 H).

### b) 1-[N-(tert-Butoxycarbonyl)-N-(pyridin-2-yl)amino]-4(4nitrophenyl)butane

To a suspension of NaH (170 mg, 4.25 mmoles) in dry DMF (10 mL) was added 2-(tert-butoxycarbonylamino)pyridine (750 mg, 3.86 mmoles) at 0° C. After 5 min the mixture was warmed to RT. After 15 min the mixture was cooled to 0° C. and 1-bromo-4-(4-nitrophenyl)butane (1.22 g, 4.73 mmoles) in dry DMF (5 mL) was added. The mixture was allowed to warm to RT as the bath warmed. After 18 hr the mixture was concentrated. The residue was taken up in H₂O (50 mL) and extracted with EtOAc (3×50 mL). The combined organic layers were dried over MgSO₄, filtered, and concentrated. The residue was chromatographed on silica gel (15% EtOAc/hexanes) to give the title compound (1.25 g, 87%) as a pale yellow oil: MS (ES) m/e 372 (M+H)⁺.

### c) 1-[N-(tert-Butoxycarbonyl)-N-(pyridin-2-yl)amino]-4-(4-aminophenyl)butane

To a suspension of 10% Pd/C (358 mg) in absolute EtOH (15 mL) was added 1-[N-(tertbutoxycarbonyl)-N-(pyridin-2-yl)amino]-4-(4-nitrophenyl)butane (1.25 g, 3.37 mmoles). The mixture was deoxygenated (3×evacuation/N₂ purge cycles) then charged with H₂ (50 psi). After 2 hr the H₂ was removed and the mixture filtered through a pad of celite®. The filtrate was concentrated to give the title compound (1.14 g, 99%) as a yellow oil which was used without purification: MS (ES) m/e 342 (M+H)⁺.

### d) tert-Butyl (±)-3-phenyl-3-[4-[4-[N-(tert-butoxycarbony)-N-(pyridin-2-yl)amino]-1-butyl]phenylamino]propanoate

To a suspension of MgSO₄ (7.0 g) in CH₂Cl₂ (20 mL) was added 1-[N-(tert-butoxycarbonyl)-N-(pyridin-2-yl)amino]-4-(4-aminophenyl)butane (560 mg, 1.64 mmoles) then benzaldehyde (0.2 mL, 1.97 mmoles). After 18 hr the mixture was filtered and the filtrate was concentrated. The residue was taken up in dry THF (10 mL) and cooled to −78 ° C. To this mixture was added BF₃.OEt₂ (0.4 mL, 3.28 mmoles) dropwise. After 15 min, the Reformatsky reagent prepared from zinc metal and tert-butyl bromoacetate in THF (Tetrahedron 1984, 40, 2781; 818 mg, 2.46 mmoles) was added. The mixture was allowed to warm to RT over 5 hr as the bath warmed. The mixture was diluted with H₂O (20 mL) and extracted with EtOAc (3×20 mL). The combined organic layers were dried over MgSO₄, filtered, and concentrated. The residue was chromatographed on silica gel (15% EtOAc/hexanes) to give the title compound (350 mg, impure): MS (ES) m/e 546 (M+H)⁺. This was used in the next step without further purification.

### e) (±)-3-Phenyl-3-[4-[4-(pyridin-2-yl)amino-1-butyl]phenylamino]propanoic acid

tert-Butyl (±)-3-phenyl-3-[4-[4-[N-(tert-butoxycarbony)-N-(pyridin-2-yl)amino]-1-butyl]phenylamino]propanoate (350 mg, impure) was dissolved in 1:1 TFA/CH₂Cl₂ (10 mL). After 2 hr the mixture was concentrated. The residue was dissolved in 1.0 M NaOH (10 mL) and extracted with EtOAc (2×10 mL). The aqueous layer was acidified to pH 6 using 10% HCl. The solid was collected by filtration and dried in vacuo at 50° C. for 18 hr to give the title compound (74 mg, 12%) as an off-white powder: MS (ES) m/e 390 (M+H)⁺. Anal. Calcd for C₂₄H₂₇N₃O₂.0.50 H₂O: C, 72.34; H, 7.08; N, 10.54. Found: C, 72.29; H, 6.92; N, 10.37.

### EXAMPLE 5

### Preparation of 4-[4-[2-[6-(methylamino)pyridin-2-yl]-1-ethoxy]phenyl]butanoic acid

### a) Methyl 4-[4-[2-[6-(methylamino)pyridin-2-yl]-1-ethoxy]phenyl]butanoate

Diisopropyl azodicarboxylate (0.3 mL, 1.4 mmole) was added to a solution of methyl 4-(4-hydroxyphenyl)butanoate (180 mg, 0.93 mmole), 6-(methylamino)-2-pyridylethanol (212 mg, 1.4 mmole), and triphenylphosphine (367 mg, 1.4 mmole) in anhydrous THF (10 mL) at 0° C. The mixture was allowed to warm to RT as the bath warmed. After 24 hr the mixture was concentrated and the residue was chromatographed on silica gel (Et₂O). The title compound (160 mg, 52%) was obtained as a pale yellow oil: ¹H NMR (300 MHz, CDCl₃) ?7.39 (t, 1 H), 7.05 (d, J=6.6 Hz, 2 H), 6.82 (d, J=6.6 Hz, 2 H), 6.52 (d, J=8 Hz, 1 H), 6.13 (d, J=8.0 Hz, 1 H), 4.51 (br s, 1 H), 4.28 (t, 2 H), 3.72 (t, 2 H), 3.65 (s, 3 H), 3.06 (t, 2 H), 2.89 (d, J=6.0 Hz, 3 H), 2.55 (t, 2 H), 2.30 (t, 2 H), 1.88 (m, 2 H).

### b) 4-[4-[2-[6-(Methylamino)pyridin-2-yl]-1-ethoxy]phenyl]butanoic acid

To a solution of methyl 4-[4-[2-[6-(methylamino)pyridin-2-yl]-1-ethoxy]phenyl]butanoate (160 mg, 0.49 mmoles) in 1:1 THF/H₂O (1.5 mL) was added 1.0 N LiOH (0.58 mL, 0.58 mmoles). After 5 hr the mixture was concentrated to remove THF. The resulting aqueous solution was cooled to 0° C. and acidified to pH 6 using 10% HCl. The title compound (94 mg, 61%) was collected by filtration and dried in vacuo at 50° C. for 18 hr: MS (ES) m/e 315 (M+H)⁺. Anal. Calcd for C₁₈H₂₂N₂O₃: C, 68.77; H, 7.05; N, 8.91. Found: C, 68.75; H, 7.06; N, 8.74.

### EXAMPLE 6

### Preparation of (±)-4-[4-[2-[6-(methylamino)pyridin-2-yl]-1-ethoxy]phenyl]-3-vinylbutanoic acid

### a) Methyl (±)-4-[4-[2-[6-(methylamino)pyridin-2-yl]-1-ethoxy]phenyl]-3-vinylbutanoate

Diisopropyl azodicarboxylate (0.17 mL, 0.84 mmole) was added to a solution of methyl (±)-4-(4-hydroxyphenyl)-3-vinylbutanoate (92.5 mg, 0.42 mmole), 6-(methylamino)-2-pyridylethanol (128 mg, 0.84 mmole), and triphenylphosphine (220 mg, 0.84 mmole) in anhydrous THF (2 mL) at 0° C. The mixture was allowed to warm to RT as the bath warmed. After 24 hr the mixture was concentrated and the residue was chromatographed on silica gel (3:1 Et₂O/hexanes). The title compound (100 mg, 67%) was obtained as a pale yellow oil: MS (ES) m/e 355 (M+H)⁺.

### b) (±)-4-[4-[2-[6-(Methylamino)pyridin-2-yl]-1-ethoxy]phenyl]-3-vinylbutanoic acid

To a solution of methyl (±)-4-[4-[2-[6-(methylamino)pyridin-2-yl]-1-ethoxy]phenyl]-3-vinylbutanoate (100 mg, 0.28 mmoles) in 1:1 THF/H₂O (1.5 mL) was added 1.0 N LiOH (0.34 mL, 0.34 mmoles). After 18 hr the mixture was acidified to pH 6 using 10% HCl and extracted with EtOAc (3×10 mL). The combined organic layers were dried over MgSO₄, filtered, and concentrated. The residue was lyophilized from HOAc (10 mL) to give the title compound (50 mg, 52%) as a yellow oil: MS (ES) m/e 341 (M+H)⁺. Anal. Calcd for C₂₀H₂₄N₂O₃.2.75 CH₃CO₂H: C, 60.58; H, 6.98; N, 5.54. Found: C, 60.55; H, 6.91; N, 5.47.

### EXAMPLE 7

### Preparation of (±)-4-[4-[2-[6-(methylamino)pyridin-2-yl]-1-ethoxy]phenyl]-3-(pyridin-2-yl)butanoic acid

### a) Ethyl (±)-4-[4-[2-[6-(methylamino)pyridin-2-yl]-1-ethoxy]phenyl]-3-(pyridin-2-yl)butanoate

Diisopropyl azodicarboxylate (0.12 mL, 0.62 mmole) was added to a solution of ethyl (±)-4-(4-hydroxyphenyl)-3-(pyridin-2-yl)butanoate (90 mg, 0.31 mmole), 6-(methylamino)-2-pyridylethanol (95 mg, 0.62 mmole), and triphenylphosphine (163 mg, 0.62 mmole) in anhydrous THF (2 mL) at 0° C. The mixture was allowed to warm to RT as the bath warmed. After 24 hr the mixture was concentrated and the residue was chromatographed on silica gel (10% hexanes/Et₂O). The title compound (71 mg, 55%) was obtained as a colorless oil: MS (ES) m/e 420 (M+H)⁺.

### b) (±)4-[4-[2-[6-(Methylamino)pyridin-2-yl]-1-ethoxy]phenyl]-3-(pyridin-2-yl)butanoic acid

To a solution of ethyl (±)-4-[4-[2-[6-(methylamino)pyridin-2-yl]-1-ethoxy]phenyl]-3-(pyridin-2-yl)butanoate (71 mg, 0.17 mmoles) in 1:1 THF/H₂O (2 mL) was added 1.0 N LiOH (0.34 mL, 0.34 mmoles). After 18 hr the mixture was acidified to pH 6 using 10% HCl and extracted with CHCl₃ (3×10 mL). The combined organic layers were dried over MgSO₄, filtered, and concentrated. The residue was chromatographed on silica gel (10% MeOH/CHCl₃) to give the title compound as a yellowish foam. MS (ES) m/e 392 (M+H)⁺. Anal. Calcd for C₂₃H₂₅N₃O₃.0.75 H₂O: C, 68.21; H, 6.60; N, 10.38. Found: C, 68.50; H, 6.39; N, 10.24.

### EXAMPLE 8

### Preparation of (±)-4-[4-[2-[6-(methylamino)pyridin-2-yl]-1-ethoxy]phenyl]-3-(oxazol-2-yl)butanoic acid

### a) Methyl (±)-4-[2-[6-(methylamino)pyridin-2-yl]-1-ethoxy]phenyl]-3-(oxazol-2-yl)butanoate

Diisopropyl azodicarboxylate (0.24 mL, 1.24 mmole) was added to a solution of methyl (±)-4-(4-hydroxyphenyl)-3-(oxazol-2-yl)butanoate (163 mg, 0.62 mmole), 6-(methylamino)-2-pyridylethanol (190 mg, 1.24 mmole), and triphenyiphosphine (325 mg 1.24 mmole) in anhydrous THF (4 mL) at 0° C. The mixture was allowed to warm as the bath warmed to RT. After 24 hr the mixture was concentrated and the residue was chromatographed on silica gel (50% EtOAc/CHCl₃). The title compound (167 mg, 68%) was obtained as an orangish oil: MS (ES) m/e 396 (M+H)⁺.

### b) (±)-4-[4-[2-[6-(Methylamino)pyridin-2-yl]-1-ethoxy]phenyl]-3-(oxazol-2-yl)butanoic acid

To a solution of methyl (±)-4-[4-[2-[6-(methylamino)pyridin-2-yl]-1-ethoxy]phenyl]-3-(oxazol-2-yl)butanoate (167 mg, 0.42 mmoles) in 1:1 THF/H₂O (4 mL) was added 1.0 N LiOH (0.63 mL, 0.63 mmoles). After 18 hr the mixture was washed with Et₂O (2×2 mL). The aqueous layer was concentrated to remove residual THF/Et₂O then was acidified to pH 6 using 10% HCl. The title compound (114 mg, 71%) was collected as a white solid by filtration and dried in vacuo at 50° C. for 18 hr. MS (ES) m/e 382 (M+H)⁺. Anal. Calcd for C₂₁H₂₃N₃O₄.0.50 H₂O: C, 64.60; H, 6.20; N, 10.76. Found: C, 64.33; H, 6.12; N, 10.38.

### EXAMPLE 9

### Preparation of (±)-4-[4-[2-[6-(methylamino)pyridin-2-yl]-1-ethoxy]phenyl]-3-(thiazol-2-yl)butanoic acid

### a) Ethyl (±)-4-[4-[2-[6-(methylamino)pyridin-2-yl]-1-ethoxy]phenyl]-3-(thiazol-2-yl)butanoate

Diisopropyl azodicarboxylate (0.21 mL, 1.06 mmole) was added to a solution of ethyl (±)-4-(4-hydroxyphenyl)-3-(thiazol-2-yl)butanoate (155 mg, 0.53 mmole), 6-(methylamino)-2-pyridylethanol (163 mg, 1.06 mmole), and triphenylphosphine (278 mg, 1.06 mmole) in anhydrous THF (5 mL) at 0° C. The mixture was allowed to warm to RT as the bath warmed. After 24 hr the mixture was concentrated and the residue was chromatographed on silica gel (50% EtOAc/CHCl₃). Fractions containing the product were concentrated and rechromatographed on silica gel (60% EtOAc/hexanes). Fractions from the second chromatography which contained the product were further purified by preparative TLC (60% EtOAc/hexanes). The title compound (106 mg, 47%) was obtained as an oil: MS (ES) m/e 426 (M+H)⁺.

### b) (±)-4-[4-[2-[6-(Methylamino)pyridin-2-yl]-1-ethoxy]phenyl]-3-(thiazol-2-yl)butanoic acid

To a solution of ethyl (±)-4-[4-[2-[6-(methylamino)pyridin-2-yl]-1-ethoxy]phenyl]-3-(thiazol-2-yl)butanoate (106 mg, 0.25 mmoles) in 1:1 THF/H₂O (5 mL) was added 1.0 N LiOH (0.37 mL, 0.37 mmoles). After 18 hr the mixture was extracted with Et₂O (2×5 mL), and the Et₂O layers were discarded. The aqueous layer was concentrated to remove residual organic solvents, then was acidified to pH 6 using 10% HCl. CH₃CN (0.5 mL) was added to the mixture to dissolve all solids. The solution was purified by C18-bond/elute chromatography (H₂O, then 20% CH₃CN/H₂O). Fractions containing the product were lyophilized to give the title compound (53 mg, 53%) as a white powder: MS (ES) m/e 398 (M+H)⁺. Anal. Calcd for C₂₁H₂₃N₃O₃S: C, 63.46; H, 5.83; N, 10.57. Found: C, 63.17; H, 6.00; N, 10.37.

### EXAMPLE 10

### Preparation of (±)3-methyl-4-[4-[3-(pyridin-2-yl)amino-1-propyloxy]phenyl]butanoic acid

### a) Ethyl (±)-3-methyl-4-[4-[3-(1-oxopyridin-2-yl)amino-1-propyloxy]phenyl]butanoate

Diisopropyl azodicarboxylate (0.3 mL, 1.5 mmole) was added over 45 sec to a solution of ethyl (±)-4-[4-hydroxyphenyl)-3-methylbutanoate (220 mg, 1.0 mmole), 2-[(3-hydroxy-1-propyl)amino]pyridine-N-oxide (252 mg, 1.5 mmole), and triphenylphosphine (390 mg, 1.5 mmole) in anhydrous DMF (22.5 mL) at 0° C. under argon. The yellow solution was kept at 0° C. for 10 min, then was warmed to RT. After 23 hr, the reaction was concentrated and the residue was reconcentrated from xylenes (2×). Silica gel chromatography (1% MeOH/CH₂Cl₂) gave the title compound (200 mg, 54%) as a yellow oil: MS (ES) m/e 373 (M+H)⁺.

### b) Ethyl (±)-3-methyl-4-[4-[3-(pyridin-2-yl)amino-1-propyloxy]phenyl]butanoate

A mixture of ethyl (±)-3-methyl-4-[4-[3-(1-oxopyridin-2-yl)amino-1-propyloxy]phenyl]butanoate (200 mg, 0.54 mmole), cyclohexene (0.6 mL, 0.54 mmole), 10% Pd/C (55 mg, 00.5 mmole), and isopropanol (10 mL) was heated at reflux under argon. The mixture was heated at reflux for another 20.5 hr, then was hot-filtered through celite®. The filter pad was washed with hot 1:1 MeOH/CHCl₃ and the filtrate was concentrated. The residue was reconcentrated from toluene, then was chromatographed on silica gel (1% MeOH/CH2Cl₂) to afford the title compound (150 mg, 78%) as a colorless oil: MS (ES) m/e 357 (M+H)⁺.

### c) (±)-3-Methyl-4-[4-[3-(pyridin-2-yl)amino-1-propyloxy]phenyl]butanoic acid

A mixture of ethyl (±)-3-methyl-4-[4-[3-(pynidin-2-yl)amino-1-propyloxy]phenyl]butanoate (150 mg, 0.42 mmole), 1.0 N LiOH (1.2 mL, 1.2 mmole), THF (4 mL), and H₂O (2.8 mL) was stirred at RT for 4 hr, then was warmed in an oil bath set at 45-50° C. After 17.5 hr, the resulting homogeneous, nearly colorless solution was cooled to RT and extracted with Et₂O (2×8 mL). The Et₂O layers were discarded. The aqueous layer was stirred with gentle warming under vacuum to remove residual organic solvents, then was filtered. The resulting aqueous solution was stirred at RT while the pH was slowly and carefully adjusted to 5.5-6.0 with 1.0 N HCl. The mixture was stirred for 0 5 hr, then the solid was collected by suction filtration and washed with plenty of H₂O Drying in high vacuum at 60° C. gave the title compound (90 mg, 65%) as a glassy solid MS (ES) m/e 328 (M+H)⁺. Anal. Calcd for C₁₉H₂₄N₂O₃.0.25 H₂O: C, 68.54; H, 7 13; N, 8.35. Found: C, 68.55; H, 7.42; N, 8.41.

### EXAMPLE 11

### Preparation of (±)-3-methyl-4-[4-[2-[6-(methylamino)pyridin-2-yl]-1-ethoxy]phenyl]butanoic acid

### a) Ethyl (±)-3-methyl-4-[4-[2-[6-(methylamino)pyridin-2-yl]-1-ethoxy]phenyl]butanoate

Diisopropyl azodicarboxylate (0.44 mL, 2.25 mmole) was added over 2 min to a solution of ethyl (±)4-(4-hydroxyphenyl)-3-methylbutanoate (378 mg, 2.25 mmole), 6-(methylamino)-2-pyridylethanol (343 mg, 2.25 mmole), and triphenylphosphine (590 mg, 2.25 mmole) in anhydrous THF (22.5 mL) at 0° C. under N₂. The yellow solution was kept at 0° C. for 10 min, then was warmed to RT. After 24 hr, the reaction was concentrated and the residue was chromatographed on silica gel (6:4 EtOAc/hexanes). The title compound (200 mg, 76%) was obtained as a colorless oil: MS (ES) m/e 357 (M+H)⁺.

### b) (±)-3-Methyl-4-[4-[2-[6-(methylamino)pyridin-2-yl]-1-ethoxy]phenyl]butanoic acid

1.0 N NaOH (1 mL, 0.898 mmole) was added dropwise to a cooled (15° C.) solution of ethyl (±)-3-methyl-4-[4-[2-[6-(methylamino)pyridin-2-yl]-1-ethoxy]phenyl]butanoate (160 mg, 0.449 mmole) in THF (3 mL), and the mixture was stirred at RT for 24 hr. The resulting solution was concentrated in vacuum and the residue was dissolved in H₂O (5 mL). The pH was adjusted to 7 with 1.0 N HCl, and the supernatant was decanted away from the gummy precipitate. Thorough drying in vacuum at 60° C. for several days gave the title compound (120 mg, 82%) as a white, foamy solid: MS (ES) m/e 328 (M+H)⁺. Anal. Calcd for C₁₉H₂₄N₂O₃: C,69.49; H, 7.37; N, 8.53. Found: C, 69.03; H, 7.27; N 8.40.

### EXAMPLE 12

### Preparation of (±)-3-methyl-4-[4-[2-[2-(methylamino)pyridin-5-yl]-1-ethoxy]phenyl]butanoic acid

### a) Ethyl (±)-3-methyl-4-[4-[2-[2-(methylamino)pyridin-5-yl]-1-ethoxy]phenyl]butanoate

Diisopropyl azodicarboxylate (0.18 mL, 0.913 mmole) was added over 2 min to a solution of ethyl (±)-4-(4-hydroxyphenyl)-3-methylbutanoate (133 mg 0.6 mmole), 2-(N-(tert-butoxycarbonyl)-N-methylamino]-5-pyridylethanol (230 mg. 0.913 mmole), and triphenylphosphine (239 mg, 0.913 mmole) in anhydrous THF (5 mL) at 0° C. under N₂. The yellow solution was kept at 0° C. for 10 min, then was warmed to RT. After 24 hr, the reaction was concentrated and the residue was chromatographed on silica gel (1% MeOH/CH₂Cl₂). The title compound (200 mg, 73%) was obtained as a colorless oil: MS (ES) m/e 456 (M+H)⁺.

### b) (±)-3-Methyl-4-[4-[2-[2-(methylamino)pyridin-5-yl]-1-ethoxy]phenyl]butanoic acid

Ethyl (±)-3-methyl-4-[4-[2-[2-(methylamino)pyridin-5-yl]-1-ethoxy]phenyl]butanoate (200 mg, 0.44 mmole) was suspended in 1.0 M HCl/dioxane (5 mL). After 2 hr., the reaction was concentrated in vacuum and the residue was reconcentrated from toluene (3×10 mL). The remaining residue was taken up in 5% Na₂CO₃ solution and extracted with CH₂Cl₂. The extracts were dried over MgSO₄, filtered, and concentrated leave an oil (50 mg). This was taken up in THF (3 mL), 1.0 N LiOH (0.28 mL, 0.28 mmole) was added, and the mixture was stirred at RT for 24 hr. The resulting solution was concentrated in vacuum and the residue was dissolved in H₂O (5 mL). The pH was adjusted to 7 with 1.0 N HCl, and the supernatant was decanted away from the gummy precipitate. Thorough drying in vacuum at 60° C. for several days gave the title compound (5 mg) as a white, foamy solid: MS (ES) m/e 328 (M+H)⁺.

### EXAMPLE 13

### Preparation of (±)-4-[4-[2-[6-(methylamino)pyridin-2-yl]-1-ethoxy]phenyl]-3-(thiophen-2-yl)butanoic acid

### a) Methyl (±)-4-[4-[2-[6-[N-(tert-butoxycarbonyl)-N-methylamino)pyridin-2-yl]-1-ethoxy]phenyl]-3-(thiophen-2-yl)butanoate

A solution of methyl (±)-4-(4-hydroxyphenyl)-3-(thiophen-2-yl)butanoate (245.1 mg, 0.89 mmole) and PPh₃ (237.6 mg, 0.91 mmole) in CH₂Cl₂ was added slowly to a solution of 6-[N-(tert-butoxycarbonyl)-N-methylamino]-2-pyridylethanol (244.1 mg, 0.97 mmole) and DEAD (0.14 mL, 0.89 mmole) in CH₂Cl₂ at 0° C. The reaction was allowed to warm to RT as the bath warmed. After 24 hours, the reaction was concentrated in vacuum, and the residue was chromatographed on silica gel (gradient: 10% EtOAc/hexanes, then 20% EtOAc/hexanes, then 50% EtOAc/hexanes) to afford the title compound (122.1 mg, 26.9%): MS (ES) m/e 510.9 (M+H)⁺.

### b) (±)-4-[4-[2-[6-(Methylamino)pyridin-2-yl]-ethoxy]phenyl]-3-(thiophen-2-yl)butanoic acid

Methyl (±)-4-[4-[2-[6-[N-(tert-butoxycarbonyl)-N-methylamino)pyridin-2-yl]-1-ethoxy]phenyl]-3-(thiophen-2-yl)butanoate (122.1 mg, 0.24 mmole) was stirred with 4 N HCl/dioxane for 2.5 hr at RT, then the reaction was concentrated, and the residue was reconcentrated from toluene (2×). Since the Boc group had not been completely removed, the residue was resubmitted to the reaction conditions. After another 1.5 hr, the reaction was concentrated, and the residue was reconcentrated from toluene. This material was dissolved in dioxane (3 mL) and THF (3 mL), and 1.0 N NaOH (2 mL, 2.0 mmole) was added. The reaction was stirred at RT for 24 hr, then was concentrated. Since ester was still present, the residue was resubmitted to the reaction conditions. After an additional 20 hr at RT, the reaction was neutralized with 1.0 N HCl and concentrated. Again, ester was still present, so the residue was resubmitted to the reaction conditions, this time with warming at 60° C. After 18 hours, the reaction was neutralized with 1.0 N HCl and concentrated in vacuum. The solid residue was reconcentrated from toluene (2×), then was taken up in 0.1% TFA/H₂O. The white precipitate that separated was collected and washed with more 0.1% TFA/H₂O. Drying in vacuum gave the title compound (92.5 mg, 83%) as a white powder: MS (ES) m/e 397.1 (M+H)⁺. Anal. Calcd for C₂₂H₂₄N₂O₃S.0.5 TFA.0.5 H₂O: C, 59.73; H, 5.56; N, 6.06. Found: C, 59.62; H, 5.40; N, 6.14.

### EXAMPLE 14

### Preparation of 2-[N-benzyl-N-[4-[2-[6-(methylamino)pyridin-2-yl]-1-ethoxy]benzyl]amino]acetic acid

### a) Ethyl 2-[N-benzyl-N-[4-[2-[6-[N-(tert-butoxycarbonyl)-N-methylamino]pyridin-2-yl]-1-ethoxy]benzyl]amino]acetate

A solution of 6-[N-(tert-butoxycarbonyl)-N-methylamino]-2-pyridylethanol (0.17 g, 0.69 mmole) and diethyl azodicarboxylate (0.11 mL, 0.70 mmole) in CH₂Cl₂ (1.5 mL) was added dropwise to a solution of ethyl 2-[N-benzyl-N-(4hydroxybenzyl)amino]acetate (0.14 g, 0.46 mmole) and Ph₃P (0.18 g, 0.69 mmole) in CH₂Cl₂ (1.5 mL) at 0° C. bath was removed and the reaction was allowed to warm to RT. After 24 h, the solvent was removed under reduced pressure. Radial chromatography (20% EtOAc/hexane, silica gel, 6 mm plate) gave the title compound (0.14 g) as a clear oil: MS (ES) m/e 534.1 (M+H)⁺.

### b) 2-[N-benzyl-N-[4-[2-[6-(methylamino)pyridin-2-yl]-1-ethoxy]benzyl]amino]acetic acid

Ethyl 2-[N-benzyl-N-[4-[2-[6-[N-(tert-butoxycarbonyl)-N-methylamino]pyridin-2-yl]-1-ethoxy]benzyl]amino]acetate (0.14 g, 0.27 mmole) was dissolved in 4 N HCl/dioxane (5 mL). The reaction was stirred for 5.5 h at RT, then the solvent was removed under reduced pressure. The residue was suspended in 1.0 N NaOH (2 mL) and MeOH (2 mL). The reaction was stirred for 18 h at RT, then the solvent was removed under reduced pressure. The residue was dissolved in H₂O and the solution was acidified to pH≈5 with 1.0 N HCl. The solvent was removed under reduced pressure. Purification by preparative HPLC (Hamilton PRP-1 column, 20% CH₃CN/H₂O containing 0.1% TFA) gave the title compound (0.40 g) as a white powder: MS (ES) m/e 406.0 (M+H)⁺. Anal. Calcd for C₂₄H₂₇N₃O₃.2.5 TFA.1.5 H₂O: C, 48.54; H, 4.56; N, 5.86. Found: C, 48.69; H, 4.24; N, 5.78.

### EXAMPLE 15

### Preparation of 2-[N-[4-[2-[6-(methylamino)pyridin-2-yl]-1-ethoxy]benzyl]-N-phenyl]amino]acetic acid

### a) Methyl 2-[N-[4-[2-[6-[N′-(tert-butoxycarbonyl)-N′-methylamino]pyridin-2-yl]-1-ethoxy]benzyl]-N-phenyl]amino]acetate

According to the procedure of Example 14 (a), except substituting methyl 2-[N-(4-hydroxybenzyl)-N-phenylamino]acetate (39 mg, 0.14 mmole) for the ethyl 2-[N-benzyl-N-(4-hydroxybenzyl)amino]acetate, the title compound (8 mg) was obtained as a clear film following radial chromatography (20% EtOAc/hexane, silica gel, 2 mm plate): MS (ES) m/e 506.0 (M+H)⁺.

### b) 2-[N-[4-[2-[6-(methylamino)pyridin-2-yl]-1-ethoxy]benzyl]-N-phenyl]amino]acetic acid

A solution of 4 N HCl in dioxane (5 mL) was added to methyl 2-[N-[4-[2-[6-(methylamino)pyridin-2-yl]-1-ethoxy]benzyl]-N-phenyl]amino]acetate (8 mg, 0.016 mmole). The reaction was stirred for 5.5 h at RT, then the solvent was removed under reduced pressure to leave a clear film. This was dissolved in 1.0 N NaOH (2 mL) and MeOH (2 mL). The reaction was stirred for 18 h at RT, then the solvent was removed under reduced pressure. Flash chromatography on a C-18 Bond Elut® column (step gradient: H₂O containing 0.1% TFA, then 20% CH₃CN/H₂O containing 0.1% TFA, then 50% CH₃CN/H₂O containing 0.1% TFA) gave the title compound (1.5 mg) as a hygroscopic, dark solid: MS (ES) m/e 392.0 (M+H)⁺.

### EXAMPLE 16

### Preparation of 2-[N-[2-methoxy-4-[2-[6-(methylamino)pyridin-2-yl]-1-ethoxy]benzyl]amino]acetic acid

### a) Methyl 2-[N-[2-methoxy-4-[2-[6-[N′-(tert-butoxycarbonyl)-N′-methylamino]pyridin-2-yl]-1-ethoxy]benzyl]amino]acetic acid

According to the procedure of Example 14 (a), except substituting methyl 2-[(4-hydroxy-2-methoxybenzyl)amino]acetate (0.48 g, 2.14 mmole) for the ethyl 2-[N-benzyl-N-(4-hydroxybenzyl)amino]acetate, the title compound (0.14 g) was obtained as a clear oil after flash chromatography on silica gel (40% EtOAc/hexane) followed by radial chromatography (5% MeOH/CHCl₃, silica gel, 6 mm plate): MS (ES) m/e 506.0 (M+H)⁺.

### b) Methyl 2-[N-[2-methoxy-4-[2-[6-(methylamino)pyridin-2-yl]-1-ethoxy]benzyl]amino]acetate

A solution of 4 N HCl in dioxane (15 mL) was added to methyl 2-[N-[2-methoxy-4-[2-[6-[N′-(tert-butoxycarbonyl)-N′-methylamino]pyridin-2-yl]-1-ethoxy]benzyl]amino]acetic acid (0.14 g, 0.30 mmole). The reaction was stirred for 2 hr at RT, then the solvent was removed under reduced pressure to leave a clear residue. This was dissolved in saturated NaHCO₃, and the solution was extracted with 10% MeOH/EtOAc. The combined organic extracts were washed with brine, dried over Na₂SO₄, and concentrated to give a pale yellow oil. Flash chromatography on silica gel (5% MeOH/CHCl₃) gave the title compound (0.11 g) as a clear oil: MS (ES) m/e 350.4 (M+H)⁺.

### c) 2-[N-[2-Methoxy-4-[2-[6-(methylamino)pyridin-2-yl]-1-ethoxy]benzyl]amino]acetic acid

To a solution of methyl 2-[N-[2-methoxy-4-[2-[6-(methylamino)pyridin-2-yl]-1-ethoxy]benzyl]amino]acetate (0.11 g, 0.30 mmole) in MeOH (3 mL) was added 1.0 N NaOH (3 mL). The reaction was stirred for 15 min at RT, then the solvent was removed under reduced pressure. The residue was dissolved in H₂O and the solution was acidified to pH≈3 with conc HCl. The solvent was removed to leave a white residue. Flash chromatography on a Waters Sep-PaK® C-18 column (step gradient: H₂O, then 15% CH₃CN/H₂O) gave the title compound (0.11 g) as a very hygroscopic white solid: MS (ES) m/e 346.4 (M+H)⁺. ¹H NMR (300 MHz, DMSO-d₆) δ7.70 (m, 1 H), 7.40 (d, J=8.3 Hz, 1 H), 6.80-6.55 (m, 4 H), 4.35 (m, 2 H), 4.05 (s, 2 H), 3.80 (s, 3 H), 3.67 (s, 2 H), 3.15 (m, 2 H), 2.95 (s, 3 H).

### EXAMPLE 17

### Preparation of 2-phenoxy-4-[5-(pyridin-2-yl)amino-1-pentyloxy]phenylacetic acid

### a) Methyl 2-phenoxy-4-[5-(1-oxopyridin-2-yl)amino-1-pentyloxy]phenylacetate

According to the procedure of Example 14 (a), except substituting methyl 2-(4-hydroxy-2-phenoxyphenyl)acetate (0.19 g, 0.74 mmole) for the ethyl 2-[N-benzyl-N-(4-hydroxybenzyl)amino]acetate, the title compound (0.35 g) was obtained as a pale yellow oil following radial chromatography (50% EtOAc/hexane, silica gel, 6 mm plate): MS (ES) m/e 506.0 (M+H)⁺.

### b) Methyl 2-phenoxy-4-[5-(pyridin-2-yl)amino-1-pentyloxy]phenylacetate

To a solution of methyl 2-phenoxy-4-[5-(1-oxopyridin-2-yl)amino-1-pentyloxy]phenylacetate (0.35 g, 0.81 mmole) and cyclohexene (0.81 mL, 8.00 mmole) in EtOH (4 mL) was added 10% Pd/C (10 mg). After 18 h at reflux, the reaction was allowed to cool to RT and the catalyst was removed by filtration. The solvent was removed under reduced pressure to leave a clear oil. Radial chromatography (5% to 10% MeOH/CHCl₃, silica gel, 6 mm plate) gave the title compound (0.23 g) as a clear oil: MS (ES) m/e 421.1 (M+H)⁺.

### c) 2-Phenoxy-4-[5-(pyridin-2-yl)amino-1-pentyloxy]phenylacetic acid

To a solution of methyl 2-phenoxy-4-[5-(pyridin-2-yl)amino-1-pentyloxy]phenylacetate (0.23 g, 0.55 mmole) in MeOH (2.5 mL) was added 1.0 N NaOH (2.5 mL). The reaction was stirred for 18 h at RT, then the solvent was removed under reduced pressure. The residue was dissolved in H₂O, and the solution was acidified to pH≈4 with conc. HCl. The aqueous layer was extracted with EtOAc and the combined organic extracts were dried over Na₂SO₄. The solvent was removed to give a pale yellow oil. Flash chromatography on silica gel (10% MeOH/CHCl₃) gave the title compound (81 mg): MS (ES) m/e 407.0 (M+H)⁺. ¹ H NMR (300 MHz, CDCl₃) δ7.78 (d, J=4.1 Hz, 1 H), 7.50 (dt, J=8.7, 1.6 Hz, 1 H), 7.20 (m, 3 H), 6.95 (m, 3 H), 6.50 (m, 4H), 3.77 (t, J=6.4 Hz, 2H), 3.59(s, 2 H), 3.13 (t, J=6.6 Hz, 2 H), 1.80-1.50 (m,6 H).

### EXAMPLE 18

### Preparation of 4-[4-[6-(methylamino)pyridin-2-yl]-1-ethoxy]-2-phenoxyphenyl]butanoic acid

### a) Methyl 4-[4-[6-(methylamino)pyridin-2-yl]-1-ethoxy]-2-phenoxyphenyl]butanoate

A solution of 2-[(6-methylamino)-2-pyridinyl]ethanol (0.07 g, 0.43 mmole) and diethyl azodicarboxylate (0.07 mL, 0.44 mmole) in CH₂Cl₂ (3 mL) was added in a dropwise manner to a solution of Ph₃P (0.11 g, 0.43 mmole) and 2-phenoxy-4-[5-(pyridin-2-yl)amino-1-pentyloxy]phenylacetic acid (0.08 g, 0.29 mmole) in CH₂Cl₂ (3 mL) at 0° C. The cooling bath was removed and the reaction was allowed to warm to RT. After 18 hr, the solvent was removed under reduced pressure and the residue was purified by radial chromatography (30% to 50% EtOAc/hexanes, silica gel, 6 mm plate) to afford the title compound (0.14 g) as an oil: MS (ES) m/e 420.9 (M+H)^{+.}

### b) 4-[4-[6-(methylamino)pyridin-2-yl]-1-ethoxy]-2-phenoxyphenyl]butanoic acid

A solution of methyl 4-[4-[6-(methylamino)pyridin-2-yl]-1-ethoxy]-2-phenoxyphenyl]butanoate (0.1 g, 0.34 mmole) and 1.0 N NaOH (2 mL) in MeOH (2 mL) and THF (sufficient to afford a homogeneous solution) was stirred at RT. After 18 h, the solvent was removed under reduced pressure. The residue was suspended in H₂O, and the mixture was acidified to pH≈3 with conc HCl. The aqueous phase was extracted with EtOAc and the combined extracts were dried over Na₂SO₄. The solvent was removed under reduced pressure to leave a white foam. Flash chromatography on silica gel (EtOAc to 10% MeOH/EtOAc) gave the title compound (0.07 g) as a white foam: MS (ES) m/e 406.9 (M+H)⁺. Anal. Calcd for C₂₄H₂₆N₂O₄.0.75 H₂O: C, 68.64; H, 6.60; N, 6.67. Found: C, 68.33; H, 6.09; N, 6.54.

### EXAMPLE 19

### Preparation of (R)-3-phenyl-4-[4-[3-(pyridin-2-yl)amino-1-propyloxy]phenyl]butanoic acid

### a) Ethyl (+)-3-phenyl-4-[4-[3-(1-oxopyridin-2-yl)amino-1-propyloxy]phenyl]butanoate

Diisopropyl azodicarboxylate (0.40 mL, 2 mmole) was added over 45 sec to a solution of ethyl (R)-4-(4-hydroxyphenyl)-3-phenylbutanoate (0.39 g, 1.4 mmole), 2-[(3-hydroxy-1-propyl)amino]pyridine-N-oxide (0.35 g, 2 mmole), and triphenylphosphine (0.54 g, 2 mmole) in anhydrous DMF (20 mL) at 0° C. under argon. The yellow solution was kept at 0° C. for 10 min, then was warmed to RT. After 23 hr, the reaction was concentrated. Silica gel chromatography (gradient: 1%-4% MeOH/CHCl₃) gave the title compound (0.30 g 51%) as a yellow oil: MS (ES) m/e 434.9 (M+H)^{+.}

### b) Ethyl (R)-3-pheny-4-[4-[3-(pyridin-2-yl)amino-1-propyloxy]phenyl]butanoate

A mixture of ethyl (R)-3-phenyl-4-[4-[3-(1-oxopyridin-2-yl)amino-1-propyloxy]phenyl]butanoate (0.30 g, 0.69 mmole), cyclohexene (1 mL, 10 mmole), 10% Pd/C (93 mg, 0.09 mmole), and isopropanol (5 mL) was heated at reflux under argon. After 3 hr, more Pd/C (110 mg) was added. The mixture was heated at reflux for another 20.5 hr, then was hot-filtered through celite®. The filter pad was washed with hot EtOAc, and the combined filtrates were concentrated to afford the title compound (0.25 g, 87%) as a pale yellow oil: MS (ES) m/e 419.1 (M+H)^{+.}

### c) (R)-3-Phenyl-4-[4-[3-(pyridin-2-yl)amino-1-propyloxy]phenyl]butanoic acid

A mixture of ethyl (R)-3-phenyl-4-[4-[3-(pyridin-2-yl)amino-1-propyloxy]phenyl]butanoate (0.25 g, 0.6 mmole)and lithium hydroxide monohydrate (32 mg, 0.76 mmole) in THF (5 mL) and H₂O (3 mL) was stirred at RT for 18 hr, then was concentrated, and the residue was dissolved in H₂O. The resulting aqueous solution was stirred at RT while the pH was slowly and carefully adjusted to 5.5-6.0 with 1.0 N HCl. The mixture was stirred for 0.5 hr, then the solid was collected by suction filtration and washed with plenty of H₂O. Drying in high vacuum at 60° C. gave the title compound (100 mg, 43%) as a glassy solid: MS (ES) m/e 390.7 (M+H)⁺. Anal. Calcd for C₂₄H₂₆N₂O₃.0.25 H₂O: C, 73.82; H, 6.71; N, 7.17. Found: C, 72.98; H, 6.76; N, 7.09.

### EXAMPLE 20

### Preparation of (S)-3-phenyl-4-[4-[2-[6-(methylamino)pyridin-2-yl]-1-ethoxy]phenyl]butanoic acid

### a) Ethyl(S)-3-phenyl -4-[4-[2-[6-(methylamino)pyridin-2-yl]-1-ethoxy]phenyl]butanoate

Diisopropyl azodicarboxylate (0.16 mL, 0.80 mmole) was added over 2 min to a solution of ethyl (S)-4-(4-hydroxyphenyl)-3-phenylbutanoate (0.19 g, .66 mmole), 6(methylamino)-2-pyridylethanol (0.12 g, 0.80 mmole), and triphenylphosphine (0.20 g 0.80 mmole) in anhydrous CH₂Cl₂ (5 mL) at 0° C. under N₂. The yellow solution was kept at 0° C. for 10 min, then was warmed to RT. After 24 hr. the reaction was concentrated and the residue was chromatographed on silica gel (gradient: 10%-30% EtOAc/hexanes). The title compound (0.26 g, 93%) was obtained as a colorless oil: MS (ES) m/e 419.0 (M+H)^{+.}

### b) (S)-3-Phenyl-4-[4-[2-[6-(methylamino)pyridin-2-yl]-1-ethoxy]phenyl]butanoic acid

Lithium hydroxide monohydrate (29 mg, 0.69 mmole) in H₂O (2 mL) was added to a solution of ethyl (S)-3-phenyl-4-[4-[2-[6-(methylamino)pyridin-2-yl]-1-ethoxy]phenyl]butanoate (0.25 g, 0.62 mmole) in THF (5 mL). The resulting mixture was stirred at RT for 18 hr, then was concentrated. The residue was dissolved in H₂O, and the pH was adjusted to 5.5-6.0 with 1.0 N HCl. The aqueous solution was decanted away from the gummy precipitate, which was dried in vacuum at 60° C. for several days to afford the title compound (0.10 g, 41%) as a white solid: MS (ES) m/e 391.0 (M+H)⁺. Anal. Calcd for C₂₄H₂₆N₂O₃: C, 73.82; H, 6.71; N, 7.17. Found: C, 73.62; H, 6.80; N, 6.98.

### EXAMPLE 21

### Preparation of (S)-3-phenyl -4-[4-[3-(pyridin-2-yl)amino-1-propyloxy]phenyl]butanoic acid

### a) Ethyl (S)-3-phenyl-4-[4-[3-(1-oxopyridin-2-yl)Boc-amino-1-propyloxy]phenyl]butanoate

Sodium hydride (80% in mineral oil, 66 mg, 2.2 mmole) was added to a solution of ethyl (S)-4-(4-hydroxyphenyl)-3-phenylbutanoate (0.60 g, 2 mmole) in anhydrous DMSO (6 mL) at 23° C. under argon. After the mixture became homogeneous, 2-[N-(3-methanesulfonyloxy-1-propyl)-N-(tert-butoxycarbonyl)amino]pyridine-N-oxide (0.35 g, 2 mmole) was added. The resulting solution was stirred at room temperature for 5 days, then was then partitioned between EtOAc and H₂O. The organic phase was washed twice with H₂O and once with brine, dried (MgSO₄), and concentrated. Silica gel chromatography (gradient: 0.5%-4% MeOH/CH₂Cl₂) gave the title compound (0.30 g, 55% based on recovered starting material) as a yellow oil: MS (ES) m/e 535.0 (M+H)⁺. Unchanged ethyl (S)-4-(4-hydroxyphenyl)-3-phenylbutanoate (0.30 g) was recovered.

### b) Ethyl-(S)-3-phenyl-4-[4-[3-(1-oxopyridin-2-yl)amino-1-propyloxy]phenyl]butanoate

A solution of ethyl (S)-3-phenyl -4-[4-[3-(1-oxopyridin-2-yl)Boc-amino-1-propyloxy]phenyl]butanoate (0.30 g, 0.56 mmole), CH₂Cl₂ (5 mL), and TFA (5 mL) was stirred at 0° C. for 1 hr, then was allowed to warm to RT. After an additional 2 hr, the solution was concentrated to afford the title compound (0.15 g) as a pale yellow oil: MS (ES) m/e 435.2 (M+H)^{+.}

### c) Ethyl (S)-3-phenyl-4-[4-[3-(pyridin-2-yl)amino-1-propyloxy]phenyl]butanoate

A mixture of ethyl (S)-3-phenyl-4-[4-[3-(1-oxopyridin-2-yl)amino-1-propyloxy]phenyl]butanoate (0.15 g, 0.35 mmole), cyclohexene (0.5 mL, 5 mmole), 10% Pd/C (80 mg, 0.075 mmole), and isopropanol (5 mL) was heated at reflux under argon. After 20.5 hr, the mixture was hot-filtered through celite®. The filter pad was washed with hot EtOAc, and the combined filtrates were concentrated to afford the title compound (0.1 g, 43%) as a pale yellow oil: MS (ES) m/e 419.2 (M+H)^{+.}

### d) (S)-3-Phenyl-4-[4-[3-(pyridin-2-yl)amino-1-propyloxy]phenyl]butanoic acid

A mixture of ethyl (S)-3-phenyl-4-[4-[3-(pyridin-2-yl)amino-1-propyloxy]phenyl]butanoate (0.10 g, 0.24 mmole) and lithium hydroxide monohydrate (12 mg, 0.29 mmole) in THF (5 mL) and H₂O (2 mL) was stirred at RT for 18 hr, then was concentrated. The residue was dissolved in H₂O, and the resulting aqueous solution was stirred at RT while the pH was slowly and carefully adjusted to 5.5-6.0 with 1.0 N HCl. The mixture was stirred for 0.5 hr, then the solution was decanted away from the solid. Drying in high vacuum at 60° C. gave the title compound (40 mg, 43%) as a glassy solid: MS (ES) m/e 390.7 (M+H)⁺. Anal. Calcd for C₂₄H₂₆N₂O₃.1.7 HCl: C, 63.72; H, 6.17; N, 6.19. Found: C, 63.56; H, 6.22; N, 6.10.

### EXAMPLE 22

### Preparation of (±)-3-(4-bromophenyl)-4-[4-[2-[6-(methylamino)pyridin-2-yl]-1-ethoxy]phenyl]butanoic acid

### a) Ethyl (±)-3-(4-bromophenyl)-4-[4-[2-[6-[N-(tert-butoxycarbonyl)-N-methylamino]pyridin-2-yl]-1-ethoxy]phenyl]butanoate

Diisopropyl azodicarboxylate (0.24 mL, 1.24 mmole) was added slowly to a solution of ethyl (±)-3-(4-bromophenyl)-4-(4-hydroxyphenyl)butanoate (0.30 g, 0.82 mmole), 6-[N-(tert-butoxycarbonyl)-N-methylamino]-2-pyridylethanol (0.31 g, 1.24 mmole); and triphenylphosphine (0.32 g, 1.24 mmole) in anhydrous CH₂Cl₂ (10 mL) at 0° C. under argon. The yellow solution was kept at 0° C. for 10 min, then was warmed to RT. After 39 hr, the reaction was concentrated and the residue was chromatographed on silica gel (20% EtOAc/hexanes) gave the title compound (0.32 g, 65%) as a clear oil: TLC R_{f} (20% EtOAc/hexanes) 0.44; MS (ES) m/e 349.1 (M+Na)⁺, 674.9 (2M+Na)^{+.}

### b) Ethyl (±)-3-(4-bromophenyl)-4-[4-[2-[6-(methylamino)pyridin-2-yl]-1-ethoxy]phenyl]butanoate

A solution of ethyl (±)-3-(4-bromophenyl)-4-[4-[2-[6-[N-(tert-butoxycarbonyl)-N-methylamino]pyridin-2-yl]-1-ethoxy]phenyl]butanoate (0.32 g, 0.53 mmole) in 4 N HCl in dioxane (15 mL) was stirred at RT for 1.5 hr. Concentration and reconcentration from CH₂Cl₂ and hexanes afforded the title compound as a white syrup which was carried forward without further purification.

### c) (±)-3-(4-Bromophenyl)-4-[4-[2-[6-(methylamino)pyridin-2-yl]-1-ethoxy]phenyl]butanoic acid

1.0 N NaOH (1.44 mL, 1.44 mmole) was added dropwise to a solution of ethyl (±)-3-(4-bromophenyl)-4-[4-[2-[6-(methylamino)pyridin-2-yl]-1-ethoxy]phenyl]butanoate (0.26 g, 0.48 mmole) in dioxane (10 mL) and H₂O (5.0 mL). The resulting mixture was stirred at 50° C. for 3 hr, then was concentrated. The residue was diluted with H₂O (5 mL), and the solution was neutralized with 1.0 N HCl. The precipitated solid was collected and dried to afford the title compound (0.20 g, 81%) as a white, crystalline solid: HPLC (Hamilton PRP-1®, gradient over 20 min: 10%-80% CH₃CN/H₂O containing 0.1% TFA) K′=13.28; Anal. Calcd for C₂₄H₂₅N₂O₃Br.1.5 HCl.0.25 H₂O: C, 54.54; H, 5.15; N, 5.30. Found: C, 54.49; H, 4.97; N, 5.10.

### EXAMPLE 23

### Preparation of (±)-3-(4-isopropylphenyl)-4-[4-[2-[6-(methylamino)pyridin-2-yl]-1-ethoxy]phenyl]butanoic acid

### a) Methyl (±)-3-(4-isopropylphenyl)-4-[4-[2-[6-(methylamino)pyridin-2-yl]-1-ethoxy]phenyl]butanoate

According to the procedure of Example 22(a), except substituting methyl (±)-4-(4-hydroxyphenyl)-3-(4-isopropylphenyl)butanoate for the ethyl (±)-3-(4-bromophenyl)-4-(4-hydroxyphenyl)butanoate, and substituting 6-(methylamino)-2-pyridylethanol for the 6-[N-(tert-butoxycarbonyl)-N-methylamino]-2-pyridylethanol, the title compound was obtained following silica gel chromatography (30% EtOAc/hexanes): MS (ES) m/e 447.0 (M+H)^{+.}

### b) (±)-3-(4-Isopropylphenyl)-4-[4-[2-[6-(methylamino)pyridin-2-yl]-1-ethoxy]phenyl]butanoic acid

According to the procedure of Example 22(c), except substituting methyl (±)-3-(4-isopropylphenyl)-4-[4-[2-[6-(methylamino)pyridin-2-yl]-1-ethoxy]phenyl]butanoate for the ethyl (±)-3-(4-bromophenyl)-4-[4-[2-[6-(methylamino)pyridin-2-yl]-1-ethoxy]phenyl]butanoate, the title compound was obtained: HPLC (Hamilton PRP-1®, gradient over 20 min: 10%-80% CH₃CN/H₂O containing 0.1% TFA) K′=14.19; MS (ES) m/e 435.5 (M+H)^{+.}

### EXAMPLE 24

### Preparation of (±)-3-(4-isopropylphenyl)-4-[4-[3-(4-methylpyridin-2-yl)amino-1-propyloxy]phenyl]butanoic acid

### a) Methyl (±)-3-(4-isopropylphenyl)-4-[4-[3-(4-methyl-1-oxopyridin-2-yl)amino-1-propyloxy]phenyl]butanoate

NaOH (0.14 g, 3.37 mmole) was added to a solution of 2-[(3-bromo-1-propyl)amino]pyridine-N-oxide (0.37 g, 1.13 mmole) and methyl (±)-4-(4-hydroxyphenyl)-3-(4-isopropylphenyl)butanoate (0.32 g, 1.02 mmole) in anhydrous CH₃CN (15 mL). After stirring at RT under argon for 20 hr. the reaction was filtered and concentrated on the rotavap. Silica gel chromatography (5% MeOH/CH₂Cl₂) gave the title compound (0.31 g, 64%) as a clear oil: MS (ES) m/e 477.1 (M+H)^{+.}

### b) Methyl (±)-3-(4-isopropylphenyl)-4-[4-[3-(4-methylpyridin-2-yl)amino-1-propyloxy]phenyl]butanoate

A mixture of methyl (±)-3-(4-isopropylphenyl)-4-[4-[3-(4-methyl-1-oxopyridin-2-yl)amino-1-propyloxy]phenyl]butanoate (0.31 g, 0.65 mmole), 10% Pd/C (0.31 g, 0.29 mmole), cyclohexene (0.66 mL, 6.51 mmole), and isopropanol (15 mL) was heated at reflux for 16 hr, then the catalyst was removed by filtration through celite®. Concentration and silica gel chromatography (5% MeOH/CH₂Cl₂) gave the title compound (0.25 g, 83%) as a light yellow oil: MS (ES) m/e 460.9 (M+H)^{+.}

### c) (±)-3-(4-Isopropylphenyl)-4-[4-[3-(4-methylpyridin-2-yl)amino-1-propyloxy]phenyl]butanoic acid

According to the procedure of Example 22(c), except substituting methyl (±)-3-(4-isopropylphenyl)-4-[4-[3-(4-methylpyridin-2-yl)amino-1-propyloxy]phenyl]butanoate for the ethyl (±)-3-(4bromophenyl)-4-[4-[2-[6-(methylamino)pyridin-2-yl]-1-ethoxy]phenyl]butanoate, the title compound was obtained: HPLC (Hamilton PRP-1®, gradient over 20 min: 10%-80% CH₃CN/H₂O containing 0.1% TFA) K′=14.57; MS (ES) m/e 447.5 (M+H)^{+.}

### EXAMPLE 25

### Preparation of 4-[4-[2-[6-(methylamino)pyridin-2-yl]-1-ethoxy]phenyl]but-3-enoic acid

### a) Methyl 4-[4-[2-[6-(methylamino)pyridin-2-yl]-1-ethoxy]phenyl]crotonate

According to the procedure of Example 5(a), except substituting methyl 4-(4-hydroxyphenyl)crotonate (0.46 g, 2.39 mmole) for the methyl 4-(4-hydroxyphenyl)butanoate, the title compound (0.6 g, 76%) was prepared: MS (ES) m/e 327 (M+H)^{+.}

### b) 4-[4-[2-[6-(Methylamino)pyridin-2-yl]-1-ethoxy]phenyl]but-3-enoic acid

1.0 N NaOH (1.8 mL, 1.8 mmole) was added to a solution of methyl 4-[4-[2-[6-(methylamino)pyridin-2-yl]-1-ethoxy]phenyl]crotonate (0.3 g, 0.92 mmole) in MeOH (5 mL). The reaction was stirred at RT overnight, then was concentrated in vacuum. Flash chromatography on silica gel (gradient: CH₂Cl₂, then 1% MeOH/CH₂Cl₂, then 1% MeOH/CH₂Cl₂ containing 0.5% HCO₂H) to afford the title compound (0.09 g, 31%) as a slightly yellow solid: MS (ES) m/e 313 (M+H)⁺; ¹H NMR (360 MHz, DMSO-d₆) ? 7.85 (app t, 1H), 7.33 (d, J=8.7 Hz, 2 H), 6.84-6.96 (m, 4 H), 6.81 (d, J=7.2 Hz, 1 H), 6.40 (d, J=16.0 Hz, 1 H), 6.08-6.18 (m, 1 H), 4.22-4.35 (m, 2 H), 3.09-3.29 (m, 4 H), 2.96 (s, 3 H). Anal. Calcd for C₁₈H₂₀N₂O₃.1.0 HCO₂H: C, 63.68; H, 6.19; N, 7.82. Found: C, 63.84; H, 6.42; N, 7.98.

### EXAMPLE 27

### Preparation of (S)-3-phenyl-4-[4-[2-(5,6,7,8-tetrahydro-1,8-naphthyridin-2-yl)-1-ethoxy]phenyl]butanoic acid

### a) Ethyl (S)-3-phenyl-4-[4-[2-(5,6,7,8-tetrahydro-1,8-naphthyridin-2-yl)-1-ethoxy]phenyl]butanoate

Diisopropyl azodicarboxylate (0.25 mL, 1.25 mmole) was added to a solution of ethyl (S)-3-phenyl-4-(hydroxyphenyl)butanoate (178 mg, 0.63 mmole), 2-(5,6,7,8-tetrahydro-1,8-naphthyridin-2-yl)ethanol (223 mg, 1.25 mmole), and triphenylphosphine (328 mg, 1.25 mmole) in anhydrous THF (5 mL) at 0° C. The mixture was allowed to warm as the bath warmed to RT. After 18 hr the mixture was concentrated and the residue was chromatographed on silica gel (4.5:1 Et₂O/hexanes) to give the title compound (197 mg, 71%) as a clear oil. MS (ES) m/e 445 (M+H)^{+.}

### b) (S)-3-Phenyl-4-[4-[2-(5,6,7,8-tetrahydro-1,8-naphthyridin-2-yl)-1-ethoxy]phenyl]butanoic acid

To a solution of ethyl (S)-3-phenyl-4-[4-[2-(5,6,7,8-tetrahydro-1,8-naphthyridin-2-yl)-1-ethoxy]phenyl]butanoate (197 mg, 0.44 mmole) in 1:1 THF/H₂O (2 mL) was added 1N LiOH (0.66 mL, 0.66 mmole). After 18 hr the mixture was heated to 50° C. After 18 hr the mixture was cooled to RT and washed with Et₂O (2×5 mL). The aqueous layer was concentrated to remove residual THF/Et₂O then acidified to pH 6 using 10% HCl. The solid was collected by filtration and dried under vacuum at 50° C. to give the title compound as a white powder (136 mg, 74%). MS (ES) m/e 417 (M+H)⁺. Anal. Calcd for C₂₆H₂₈N₂O₃.0.5 H₂O: C, 73.39; H, 6.87; N, 6.58. Found: C, 73.14; H, 6.64; N, 6.26.

### EXAMPLE 28

### Preparation of (±)-3-[1-(dimethylaminosulfonyl)imidazol-2-yl]-4-[4-[2-[6-(methylamino)pyridin-2-yl]-1-ethoxy]phenyl]butanoic acid

### a) Ethyl (±)-3-[1-(dimethylaminosulfonyl)imidazol-2-yl]-4-[4-[2-[6-(methylamino)pyridin-2-yl]-1-ethoxy]phenyl]butanoate

According to the procedure of Example 9(a), except substituting ethyl (±)-3-[1-(dimethylaminosulfonyl)imidazol-2-yl]-4-(4-hydroxyphenyl)butanoate (436 mg, 1.14 mmole) for the ethyl (±)-4-(4-hydroxyphenyl)-3-(thiazol-2-yl)butanoate, the title compound (411 mg, 70%) was prepared as a light orange oil: MS (ES) m/e 516 (M+H)^{+.}

### b) (±)-3-[1-(Dimethylaminosulfonyl)imidazol-2-yl]-4-[4-[2-[6-(methylamino)pyridin-2-yl]-1-ethoxy]phenyl]butanoic acid

According to the procedure of Example 9(b), except substituting ethyl (±)-3-[1-(dimethylaminosulfonyl)imidazol-2-yl]-4-[4-[2-[6-(methylamino)pyridin-2-yl]-1-ethoxy]phenyl]butanoate (200 mg, 0.39 mmole) for the ethyl (±)-4-[4-[2-[6-(methylamino)pyridin-2-yl]-1-ethoxy]phenyl]-3-(thiazol-2-yl)butanoate, the title compound (70 mg, 37%) was prepared as a white solid: MS (ES) m/e 488 (M+H)⁺. Anal. Calcd for C₂₃H₂₉N₅O₅S.0.5 H₂O.HCl: C, 51.83; H, 5.86; N, 13.14. Found: C, 51.88; H, 5.69; N, 12.75.

### EXAMPLE 29

### Preparation of (±)-3-(imidazol-2-yl)-4-[4-[2-[6-(methylamino)pyridin-2-yl]-1ethoxy]phenyl]butanoic acid

### a) (±)-3-(Imidazol-2-yl)-4-[4-[2-[6-(methylamino)pyridin-2-yl]-1-ethoxy]phenyl]butanoic acid

Ethyl (±)-3-[1-(dimethylaminosulfonyl)imidazol-2-yl]-4-[4-[2-[6-(methylamino)pyridin-2-yl]-1-ethoxy]phenyl]butanoate (200 mg, 0.39 mmole) was dissolved in 2.0 M HCl (10 mL) and the solution was heated to reflux. After 6 hr the mixture was cooled to RT and the pH was adjusted to 6 using 1.0 N NaOH. The resulting solution was concentrated to approximately 2 mL, and was chromatographed on a C-18 bond/elute column (H₂O then 20% CH₃CN/H₂O). Fractions containing the product were combined and lyophilized to give the title compound (80 mg, 54%) as a white powder: MS (ES) m/e 381 (M+H)⁺. Anal. Calcd for C₂₁H₂₄N₄O₃.0.85 HCl: C, 61.31; H, 6.09; N, 13.62. Found: C, 61.26; H, 6.09; N, 13.62.

### EXAMPLE 30

### Preparation of (S)-4-[4-[2-[6-(methylamino)pyridin-2-yl]-1-ethoxy]phenyl]-3-(thiazol-2-yl)butanoic acid

### a) Ethyl (S)-4-[4-[2-[6-(methylamino)pyridin-2-yl]-1-ethoxy]phenyl]-3-(thiazol-2-yl)butanoate

According to the procedure of Example 9(a), except substituting ethyl (S)-4-(4-hydroxyphenyl)-3-(thiazol-2-yl)butanoate (200 mg, , 0.69 mmole) for the ethyl (±)-4-(4-hydroxyphenyl)-3-(thiazol-2-yl)butanoate, the title compound (262 mg, 89%) was prepared as a pale orange oil following silica gel chromatography (35% THF in 1:1 toluene/hexanes): MS (ES) m/e 426 (M+H)^{+.}

### b) (S)-4-[4-[2-[6-(Methylamino)pyridin-2-yl]-1-ethoxy]phenyl]-3-(thiazol-2-yl)butanoic acid

According to the procedure of Example 9(b), except substituting ethyl (S)-4-[4-[2-[6-(methylamino)pyridin-2-yl)-1-ethoxy]phenyl]-3-(thiazol-2-yl)butanoate (262 mg, 0.62 mmole) for the ethyl (±)-4-[4-[2-[6-(methylamino)pyridin-2-yl-1]-ethoxy]phenyl]-3-(thiazol-2-yl)butanoate, the title compound (112 mg, 45%) was prepared as a white solid: MS (ES) m/e 398 (M+H)⁺. Anal. Calcd for C₂₁ H₂₃N₃O₃.0.75 H₂O: C, 61.37; H, 6.01; N, 10.22. Found: C, 61.51; H, 5.89; N, 10.18.

### EXAMPLE 31

### Preparation of (R)-4-[4-[2-[6-(methylamino)pyridin-2-yl]-1-ethoxy]phenyl]-3-(thiazol-2-yl)butanoic acid

### a) Ethyl (R)-4-[4-[2-[6-(methylamino)pyridin-2-yl]-1-ethoxy]phenyl]-3-(thiazol-2-yl)butanoate

According to the procedure of Example 9(a), except substituting ethyl (R)-4-(4-hydroxyphenyl)-3-(thiazol-2-yl)butanoate (200 mg, 0.69 mmole) for the ethyl (±)-4-(4-hydroxyphenyl)-3-(thiazol-2-yl)butanoate, the title compound (265 mg, 90%) was prepared as a pale orange oil following silica gel chromatography (35% THF in 1:1 toluene/hexanes): MS (ES) m/e 426 (M+H)^{+.}

### b) (R)-4-[4-[2-[6-(Methylamino)pyridin-2-yl]-1-ethoxy]phenyl]-3-(thiazol-2-yl)butanoic acid

According to the procedure of Example 9(b), except substituting ethyl (R)-4-[4-[2-[6-(methylamino)pyridin-2-yl]-1-ethoxy]phenyl]-3-(thiazol-2-yl)butanoate (265 mg, 0.62 mmole) for the ethyl (±)-4-[4-[2-[6-(methylamino)pyridin-2-yl]-1-ethoxy]phenyl]-3-(thiazol-2-yl)butanoate, the title compound (98 mg, 40%) was prepared as a white solid: MS (ES) m/e 398 (M+H)⁺. Anal. Calcd for C₂₁H₂₃N₃O₃.0.5 H₂O: C, 62.05; H, 5.95; N, 10.34. Found: C, 62.25; H, 5.80; N, 10.37.

### EXAMPLE 32

### Preparation of (±)-3-(benzothiazol-2-yl)-4-[4-[2-[6-(methylamino)pyridin-2-yl]-1-ethoxy]phenyl]butanoic acid

### a) Ethyl (±)-3-(benzothiazol-2-yl)-4-[4-[2-[6-(methylamino)pyridin-2-yl]-1-ethoxy]phenyl]butanoate

According to the procedure of Example 9(a), except substituting ethyl (±)-3-(benzothiazol-2-yl)-4-(4-hydroxyphenyl)butanoate (200 mg, 0.59 mmole) for the ethyl (±)-4-(4-hydroxyphenyl)-3-(thiazol-2-yl)butanoate, the title compound (220 mg, 78%) was prepared as a clear oil following silica gel chromatography (60% EtOAc/hexanes): MS (ES) m/e 476 (M+H)^{+.}

### b) (±)-3-(Benzothiazol-2-yl]-4-[4-[2-[6-(methylamino)pyridin-2-yl]-1-ethoxy]phenyl]butanoic acid

According to the procedure of Example 9(b), except substituting ethyl (±)-3-(benzothiazol-2-yl)-4-[4-[2-[6-(methylamino)pyridin-2-yl]-1-ethoxy]phenyl]butanoate (220 mg, 0.46 mmole) for the ethyl (±)-4-[4-[2-[6-(methylamino)pyridin-2-yl]-1-ethoxy]phenyl]-3-(thiazol-2-yl)butanoate, the title compound (125 mg, 61%) was obtained as a white solid: MS (ES) m/e 448 (M+H)⁺. Anal. Calcd for C₂₅H₂₅N₃O₃S.0.75 H₂O: C, 65.13; H, 5.79; N, 9.11. Found: C, 65.22; H, 5.49; N, 8.92.

### EXAMPLE 33

### Preparation of (S)-4-[4-[2-(5,6,7,8-tetrahydro-1,8-naphthyridin-2-yl)-1-ethoxy]phenyl]-3-(thiazol-2-yl)butanoic acid

### a) Ethyl (S)-4-[4-[2-(5,6,7,8-tetrahydro-1,8-naphthyridin-2-yl)-1-ethoxy]phenyl]-3-(thiazol-2-yl)butanoate

According to the procedure of Example 27(a), except substituting ethyl (S)-4-(4-hydroxyphenyl)-3-(thiazol-2-yl)butanoate (200 mg, 0.69 mmole) for the ethyl (S)-3-phenyl-4-(hydroxyphenyl)butanoate, the title compound (371 mg, impure) was obtained as a clear oil following silica gel chromatography (40% THF in 1:1 CHCl₃/hexanes): MS (ES) m/e 452 (M+H)^{+.}

### b) (S)-4-[4-[2-(5,6,7,8-Tetrahydro-1,8-naphthyridin-2-yl)-1-ethoxy]phenyl]-3-(thiazol-2-yl)butanoic acid

Ethyl (S)-4-[4-[2-(5,6,7,8-tetrahydro-1,8-naphthyridin-2-yl)-1-ethoxy]phenyl]-3-(thiazol-2-yl)butanoate (371 mg, impure) was dissolved in 1:1 THF/H₂O (5 mL). To this solution was added 1.0 N LiOH (1.04 mL, 1.04 mmole) and the mixture was heated to 50° C. After 18 hr the mixture was cooled to RT and washed with Et₂O (2×5 mL). The aqueous layer was concentrated under vacuum to remove residual organic solvents, then was acidified to pH 6 using 10% HCl. The solid was collected by filtration and dried under vacuum at 50° C. to give the title compound (106 mg, 36% over 2 steps) as a white powder: MS (ES) m/e 424 (M+H)⁺. Anal. Calcd for C₂₃H₂₅N₃O₃S.0.33 HCl: C, 63.42; H, 5.86; N, 9.65. Found: C, 63.19; H, 5.61; N, 9.45.

### EXAMPLE 34

### Preparation of (±)-3-(4-Methylthiazol-2-yl)-4-[4-[2-[6-(methylamino)pyridin-2-yl 1-ethoxy]phenyl]butanoic acid

### a) Ethyl (±)-3-(4-methylthiazol-2-yl)-4-[4-[2-[6-(methylamino)pyridin-2-yl]-1-ethoxy]phenyl]butanoate

According to the procedure of Example 9(a), except substituting ethyl (±)-3-(4-methylthiazol-2-yl)-4-(4-hydroxyphenyl)butanoate (216 mg, 0.74 mmole) for the ethyl (±)-4-(4hydroxyphenyl)-3-(thiazol-2-yl)butanoate, the title compound (395 mg, impure) was prepared as a clear oil following silica gel chromatography (50% EtOAc/hexanes): MS (ES) m/e 426 (M+H)^{+.}

### b) (±)-3-(4-Methylthiazol-2-yl]-4-[4-[2-[6-(methylamino)pyridin-2-yl]-1-ethoxy]phenyl]butanoic acid

Impure ethyl (±)-3-(4methylthiazol-2-yl)-4-[4-[2-[6-(methylamino)pyridin-2-yl]-1-ethoxy]phenyl]butanoate (395 mg) was dissolved in 1:1 THF/H₂O (5 mL). To this solution was added 1.0 N LiOH (1.11 mL, 1.11 mmole), and the mixture was heated at 50° C. After 18 hr the mixture was cooled to RT and washed with Et₂O (2×5 mL). The aqueous layer was concentrated under vacuum to remove residual organic solvents, then was acidified to pH 6 using 10% HCl. The solid was collected by filtration and dried under vacuum at 50° C. to give the title compound (88 mg, 29% over 2 steps) as a pale yellow powder: MS (ES) m/e 412 (M+H)⁺. Anal. Calcd for C₂₂H₂₅N₃O₃S.0.25 HCl: C, 62.82; H, 6.05; N, 9.99. Found: C, 62.94; H, 5.95; N, 9.95.

### EXAMPLE 35

### Preparation of (±)-3-[4-carboxy-1,3-oxazol-2-yl]-4-[4-[2-[6-(methylamino)pyridin-2-yl]-1-ethoxy]phenyl]butanoic acid

### a) (±)-3-[4-Carboxy-1,3-oxazol-2-yl]-4-[4-[2-[6-(methylamino)pyridin-2-yl]-1-ethoxy]phenyl]butanoic acid

To a solution of methyl (±)-3-[4-(benzyloxycarbonyl)-1,3-oxazol-2-yl]-4-[4-[2-[6-(methylamino)pyridin-2-yl]-1-ethoxy]phenyl]butanoate (50 mg, 0.09 mmole) in 1:1 THF/H₂O (1 mL) at RT was added 1.0 N LiOH (0.28 mL, 0.28 mmole). After 72 hr the mixture was acidified to pH 6 using 10% HCl then was concentrated to dryness. The residue was purified by reverse-phase HPLC (gradient: 10-80% CH₃CN/H₂O containing 0.1% TFA). The fractions containing the product were combined and concentrated to remove CH₃CN. The resulting aqueous solution was lyophilized to give the title compound (36 mg, 94%) as a white solid: MS (ES) m/e 426 (M+H)⁺. Anal. Calcd for C₂₂H₂₃N₃O₆.1.7 TFA: C, 49.26; H, 4.02; N, 6.79. Found: C, 49.30; H, 4.24; N, 6.97.

### EXAMPLE 36

### Preparation of (±)-3-[4-(Aminocarbonyl)-1,3-oxazol-2-yl]-4-[4-[2-[6-(methylamino)pyridin-2-yl]-1-ethoxy]phenyl]butanoic acid

### a) Methyl (±)-3-[4-(aminocarbonyl)-1,3-oxazol-2-yl]-4-[4-[2-[6-(methylamino)pyridin-2-yl]-1-ethoxyphenyl]butanoate

To a solution of methyl (±)-3-[4-carboxy-1,3-oxazol-2-yl]-4-[4-[2-[6-(methylamino)pyridin-2-yl]-1-ethoxy]phenyl]butanoate (82 mg, 0.19 mmole) in dry DMF (2 mL) at RT was added NH₄Cl (30 mg, 0.56 mmole), HOBt (30 mg, 0.22 mmole), Et₃N (0.08 mL, 0.56 mmole), and EDC (42 mg, 0.22 mmole). After 18 hr the mixture was concentrated. The residue was taken up in H₂O (10 mL) and extracted with CH₂Cl₂ (3×30 mL). The combined organic layers were dried over MgSO₄ and concentrated to give the title compound (46 mg, 55%) as a light yellow oil: MS (ES) m/e 439 (M+H)^{+.}

### b) (±)-3-[4-(Aminocarbonyl)oxazol-2-yl]-4-[4-[2-[6-(methylamino)pyridin-2-yl]-1-ethoxy]phenyl]butanoic acid

According to the procedure of Example 35(a), except substituting methyl (±)-3-[4-(aminocarbonyl)-1,3-oxazol-2-yl]-4-[4-[2-[6-(methylamino)pyridin-2-yl]-1-ethoxy]phenyl]butanoate (46 mg, 0.1 mmole) for the methyl (±)-3-[4-(benzyloxycarbonyl)-1,3-oxazol-2-yl]-4-[4-[2-[6-(methylamino)pyridin-2-yl]-1-ethoxy]phenyl]butanoate, the crude product was prepared. This was purified by reverse-phase HPLC (gradient: 15-50% CH₃CN/H₂O containing 0.1% TFA). The fractions containing the product were combined and concentrated to remove CH₃CN. The resulting aqueous solution was lyophilized to give the title compound (19 mg, 45%) as a white solid: MS (ES) m/e 425 (M+H)⁺. Anal. Calcd for C₂₂H₂₄N₄O₅.2.5 TFA, 1.0 H₂O: C, 44.58; H, 3.95; N, 7.70. Found: C, 44.24; H, 3.60; N, 7.83.

### EXAMPLE 37

### Preparation of (±)-3-[4-(dimethylaminocarbonyl)-1,3-oxazol-2-yl]-4-[4-[2-[6-(methylamino)pyridin-2-yl]-1-ethoxy]phenyl]butanoic acid

### a) Methyl (±)-3-[4-(dimethylaminocarbonyl)-1,3-oxazol-2-yl]-4-[4-[2-[6-(methylamino)pyridin-2-yl]-1-ethoxy]phenyl]butanoate

To a solution of methyl (±)-3-[4-carboxy-1,3-oxazol-2-yl]-4-[4-[2-[6-(methylamino)pyridin-2-yl]-1-ethoxy]phenyl]butanoate (82 mg, 0.19 mmole) in dry DMF (2 mL) at RT was added dimethylamine hydrochloride (46 mg, 0.56 mmole), HOBt (30 mg, 0.22 mmole), Et₃N (0.08 mL, 0.56 mmole), and EDC (42 mg, 0.22 mmole). After 18 hr the mixture was concentrated. The residue was taken up in H₂O (10 mL) and extracted with CH₂Cl₂ (3×30 mL). The combined organics were dried over MgSO₄ and concentrated to the title compound (79 mg, 89%) as a light yellow oil: MS (ES) m/e 439 (M+H)^{+.}

### b) (±)-3-[4-(Dimethyiaminocarbonyl)-1,3-oxazol-2-yl]-4-[4-[2-[6-(methylamino)pyridin-2-yl]-1-ethoxy]phenyl]butanoic acid

According to the procedure of Example 35(a), except substituting methyl (±)-3-[4-(dimethylaminocarbonyl)-1,3-oxazol-2-yl]-4-[4-[2-[6-(methylamino)pyridin-2-yl]-1-ethoxy]phenyl]butanoate (79 mg, 0.17 mmole) for the methyl (±)-3-[4-(benzyloxycarbonyl)-1,3-oxazol-2-yl]-4-[4-[2-[6-(methylamino)pyridin-2-yl]-1-ethoxy]phenyl]butanoate, the crude product was prepared. This was purified by reverse-phase HPLC (gradient: 10-80% CH₃CN/H₂O containing 0.1% TFA). The fractions containing the product were combined and concentrated to remove CH₃CN. The resulting aqueous solution was lyophilized to give the title compound (48 mg, 62%) as a white solid: MS (ES) m/e 453 (M+H)⁺. Anal. Calcd for C₂₄H₂₈N₄O₅.1.8 TFA: C, 50.44; H, 4.57; N, 8.52. Found: C, 50.19; H, 4.79; N, 8.88.

### EXAMPLE 38

### Preparation of (S)-3-phenyl -4-[4-[3-(3,4,5,6-tetrahydropyrimidin-2-yl)amino-1-propyloxy]phenyl]butanoic acid

### a) Ethyl (S)-3-phenyl-4-[4-[3-(tert-butoxycarbonyl)amino-1-propyloxy]phenyl]butanoate

A solution of 3-N-(tert-butoxycarbonyl)amino-1-propanol (499 mg, 2.85 mmole) and diisopropyl azodicarboxylate (0.561 mL, 2.85 mmole) in anhydrous CH₂Cl₂ (14 mL) was added dropwise over 10 min to a solution of ethyl (S)-3-phenyl-4-(4-hydroxyphenyl)butanoate (323 mg, 1.14 mmole) and triphenylphosphine (747 mg, 2.85 mmole) in anhydrous CH₂Cl₂ (5.7 mL) at 0° C. under argon. The yellow solution was kept at 0° C. for 10 min, then was warmed to RT. After 23 hr, the reaction was concentrated on the rotavap and the residue was flash chromatographed on silica gel (15% EtOAc/hexanes) to afford the title compound (378 mg, 75%) as a white solid: ¹H NMR (300 MHz, CDCl₃) δ7.28-7.10 (m, 5 H), 6.95-6.90 (d, 2 H), 6.76-6.72 (d, 2 H), 6.84-4.70 (br s, 1 H), 4.01-3.94 (dd, 4 H), 3.38-3.27 (m, 3 H), 2.85-2.83 (d, 2 H), 2.63-2.58 (t, 2 H), 1.96-1.92 (m, 2 H), 1.43 (s, 9 H), 1.12-1.08 (t, 3 H).

### b) Ethyl (S)-3-phenyl-4-[4-(3-amino-1-propyloxy)phenyl]butanoate

4 N HCl in dioxane HCl (4.25 mL, 17 mmole) was added dropwise to a solution of ethyl (S)-3-phenyl-4-[4-[3-(tert-butoxycarbonyl)amino-1-propyloxy]phenyl]butanoate (377 mg, 0.85 mmole) at RT, and the resulting mixture was stirred for 2 hr. The solvent was removed on the rotavap and the residue was triturated with ether to afford the title compound a white solid: MS (ES) m/e 341.9 (M+H)^{+.}

### c) Ethyl (S)-3-phenyl-4-[4-[3-(pyrimidin-2-yl)amino-1-propyloxy]phenyl]butanoate

A mixture of ethyl (S)-3-phenyl-4-[4-(3-amino-1-propyloxy)phenyl]butanoate (0.85 mmole, crude), 2-bromopyrimidine (177 mg, 1.11 mmole), and NaHCO₃ (357 mg, 4.25 mmole) in EtOH (10 mL) was heated at reflux for 22 hr. The mixture was cooled to RT and the salts were removed by filtration. The filter cake was washed with EtOH. The combined filtrate and washings were concentrated on the rotavap and the residue was flash chromatographed on silica gel (25% EtOAc/hexanes) to give the title compound (289 mg, 80%, 2 steps): MS (ES) m/e 419.9 (M+H)^{+.}

### d) Ethyl (S)-3-phenyl-4-[4-[3-(3,4,5,6-tetrahydropyrimidin-2-yl)amino-1-propyloxy]phenyl]butanoate

A mixture of ethyl (S)-3-phenyl-4-[4-[3-(pyrimidin-2-yl)amino-1-propyloxy]phenyl]butanoate (286 mg, 0.68 mmole), glacial HOAc (10 mL), conc. HCl (0.113 mL, 1.36 mmole), and 10% Pd/C (72 mg, 0.068 mmole) was shaken at RT under H₂ (45 psi) on a Parr apparatus. After 4 hr, the reaction was filtered and concentrated to yield the title compound (240 mg, 83%): MS (ES) m/e 423.8 (M+H)^{+.}

### e) (S)-3-Phenyl-4-[4-[3-(3,4,5,6-tetrahydropyrimidin-2-yl)amino-1-propyloxy]phenyl]butanoic acid

A mixture of ethyl (S)-3-phenyl-4-[4-[3-(3,4,5,6-tetrahydropyrimidin-2-yl)amino-1-propyloxy]phenyl]butanoate (240 mg, 0.56 mmole), 1.0 N NaOH (1.15 mL, 1.12 mmole), THF (4 mL), and EtOH (4 mL) was stirred in an oil bath set at 35° C. After 18 hr, the mixture was cooled to RT and washed with Et₂O (2×5 mL). The Et₂O washings were discarded. The remaining aqueous layer was concentrated briefly on the rotavap to remove residual organic solvents, then was filtered, and the filtrate was acidified to pH 5 with 30% TFA. Preparative HPLC (Hamilton PRP-1®, 250×21.5 mm, 35% CH₃CN/H₂O containing 0.1% TFA) followed by lyophilization gave the title compound (80 mg) as a white powder: MS (ES) m/e 395.9 (M+H)⁺. Anal. Calcd for C₂₃H₂₉N₃O₃.TFA: C, 58.93; H, 5.93; N, 8.25. Found: C, 58.63; H, 5.59; N, 7.99.

### EXAMPLE 39

### Preparation of (±)-3-[4-[2-[6-(methylamino)pyridin-2-yl]ethoxy]benzyl]pent-4-ynoic acid

### a) Methyl (±)-3-[4-[2-[6-[N-(tert-butoxycarbonyl)methylamino]pyridin-2-yl]ethoxy]benzyl]pent-4-ynoate

To a solution of methyl (±)-3-(4-hydroxybenzyl)pent-4-ynoate (25 mg, 0.12 mmole), 6-[(tert-butoxycarbonyl)methylamino]-2-pyridylethanol (43 mg, 0.17 mmole), Ph₃P (45 mg, 0.17 mmole) in CH₂Cl₂ (5 mL) at 0° C. was added dropwise DEAD (0.03 mL, 0.19 mmole). The reaction was allowed to warm to RT. After 2 days, the solvent was removed under reduced pressure. Radial chromatography on silica gel (2 mm plate, 20% EtOAc/hexane) gave the title compound (30 mg) as a clear oil: MS(ES) m/e 453.1 (M+H)^{+.}

### b) (±)-3-[4-[2-[6-(Methylamino)pyridin-2-yl]ethoxy]benzyl]pent-4-ynoic acid

A solution of 4 N HCl/dioxane (1 mL) was added to methyl (±)-3-[4-[2-[6-[N-(tert-butoxycarbonyl)methylamino]pyridin-2-yl]ethoxy]benzyl]pent-4-ynoate (30 mg, 0.06 mmole). After 8 hr, the solvent was removed under reduced pressure to give a pale yellow residue.

A solution of this residue, 1.0 N NaOH (0.5 ml), MeOH (0.5 mL), and THF (1 drop) was stirred at RT for 18 hr, then was concentrated to dryness under reduced pressure. The residue was dissolved in H₂O (3 mL), and the pH was adjusted to 6 with 1.0 N HCl. The aqueous layer was extracted with 10% MeOH/CHCl₃. The combined organic extracts were dried over Na₂SO₄ and the solvent was removed. The residue was lyophilized from water to give the title compound (21 mg) as a white powder. ¹H NMR (300 MHz, CDCl₃) δ7.57 (m, 1 H), 7.12 (d, J=8.5 Hz, 2 H), 6.76 (d, J=8.5 Hz, 2 H), 6.55 (d, J=7.2 Hz, 1 H), 6.40 (d, J=8.8 Hz, 1 H), 4.2 (m, 2 H), 3.70 (m, 2 H), 3.15 (m, 2 H), 2.88 (s, 3 H), 2.80 (m, 1 H), 2 70 (m, 1 H), 2.50 (m, 2 H), 2.01 (d, J=2.3 Hz, 1 H). MS (ES) m/e 339.2 (M+H)^{+.}

### EXAMPLE 40

### Preparation of (±)-4-[4-[2-[6-(methylamino)pyridin-2-yl]-1-ethoxy]phenyl]-3-(2-phenylethyl)butanoic acid

### a) Methyl (±)-4-[4-[2-[6-(methylamino)pyridin-2-yl]-1-ethoxy]phenyl]-3-(2-phenylethyl)butanoate

According to the procedure of Example 2(a), except substituting methyl (±)-4-(4-hydroxyphenyl)-3-(phenylethyl)butanoate for the ethyl (±)-4-(4-hydroxyphenyl)-3-phenylbutanoate, the title compound (59%) was obtained as a clear film following silica gel chromatography (20% EtOAc/hexanes): MS (ES) m/e 433 (M+H)^{+.}

### b) (±)-4-[4-[2-[6-(Methylamino)pyridin-2-yl]-1-ethoxy]phenyl]-3-(2-phenylethyl)butanoic acid

According to the procedure of Example 2(b), except substituting methyl (±)-4-[4-[2-[6-(methylamino)pyridin-2-yl]-1-ethoxy]phenyl]-3-(2-phenylethyl)butanoate for the ethyl (±)-3-phenyl-4-[4-[2-[6-(methylamino)pyridin-2-yl]-1-ethoxy]phenyl]butanoate, the title compound (70%) was obtained as a white foam: MS (ES) m/e 419 (M+H)⁺. Anal. Calcd for C₂₆H₃₀N₂O₃.1.1 H₂O: C, 71.24; H, 7.40; N, 6.39. Found: C, 71.29; H, 7.19; N, 6.33.

### EXAMPLE 41

### Preparation of (±)-3-benzyl-4-[4-[2-[6-(methylamino)pyridin-2-yl]-1-ethoxy]phenyl]butanoic acid

### a) Methyl (±)-3-benzyl-4-[4-[2-[6-(methylamino)pyridin-2-yl]-1-ethoxy]phenyl]butanoate

According to the procedure of Example 2(a), except substituting methyl (±)-4-(4-hydroxyphenyl)-3-benzylbutanoate for the ethyl (±)-4-(4-hydroxyphenyl)-3-phenylbutanoate, the title compound (47%) was obtained as a clear film following chromatography on silica gel (20% EtOAc/hexanes): MS (ES) m/e 419 (M+H)^{+.}

### b) (±)-4-[4-[2-[6-(Methylamino)pyridin-2-yl]-1-ethoxy]phenyl]-3-(2-benzyl)-butanoic acid

According to the procedure of Example 2(b), except substituting methyl (±)-3-benzyl-4-[4-[2-[6-(methylamino)pyridin-2-yl]-1-ethoxy]phenyl]butanoate for the ethyl (±)-3-phenyl-4-[4-[2-[6-(methylamino)pyridin-2-yl]-1-ethoxy]phenyl]butanoate, the title compound (47%) was obtained as a light yellow foam: MS (ES) m/e 405 (M+H)⁺. Anal. Calcd for C₂₅H₂₈N₂O₃.1.0 HCl.0.45 H₂O: C, 66.87; H, 6.71; N, 6.24. Found: C, 66.68; H, 6.62; N, 6.64.

### EXAMPLE 42

### Preparation of (±)-4-[4-[2-[6-(methylamino)pyridin-2-yl]-1-ethoxy]phenyl]-3-(2-cyclopropyl)-butanoic acid

### a) Methyl (±)-4-[4-[2-[6-(methylamino)pyridin-2-yl]-1-ethoxy]phenyl]-3-(2-cyclopropyl)-butanoate

According to the procedure of Example 2(a), except substituting methyl (±)-4-(4-hydroxyphenyl)-3-cyclopropylbutanoate for the ethyl (±)-4-(4-hydroxyphenyl)-3-phenylbutanoate, the title compound (64%) was obtained as a clear film following chromatography on silica gel (20% EtOAc/hexanes): MS (ES) m/e 369 (M+H)^{+.}

### b) (±)-4-[4-[2-[6-(Methylamino)pyridin-2-yl]-1-ethoxy]phenyl]-3-(2-cyclopropyl)-butanoic acid

According to the procedure of Example 2(b), except substituting methyl (±)-4-[4-[2-[6-(methylamino)pyridin-2-yl 1-1-ethoxy]phenyl]-3-(2-cyclopropyl)-butanoate for the ethyl (±)-3-phenyl-4-[4-[2-[6-(methylamino)pyridin-2-yl]-1-ethoxy]phenyl]butanoate, the title compound was obtained (9 mg) as a light yellow foam: MS (ES) m/e 355 (M+H)^{+.}

### EXAMPLE 43

### Preparation of 3-methyl-4-[4-[2-[6-(methylamino)pyridin-2-yl]-1-ethoxy]phenyl]-3-butenoic acid

### a) Methyl 3-methyl-4-[4-[2-[6-(methylamino)pyridin-2-yl]-1-ethoxy]phenyl]-3-butenoate

According to the procedure of Example 2(a), except substituting ethyl 4-(4-hydroxyphenyl)-3-methyl-3-butenoate for the ethyl (±)-4-(4hydroxyphenyl)-3-phenylbutanoate, the title compound (96%) was obtained as a clear film following chromatography on silica gel (20% EtOAc/hexanes): MS (ES) m/e 355 (M+H)^{+.}

### b) 3-Methyl-4-[4-[2-[6-(methylamino)pyridin-2-yl]-1-ethoxy]phenyl]-3-butenoic acid

According to the procedure of Example 2(b), except substituting methyl 3-methyl-4-[4-[2-[6-(methylamino)pyridin-2-yl]-1-ethoxy]phenyl]-3-butenoate for the ethyl (±)-3-phenyl-4-[4-[2-[6-(methylamino)pyridin-2-yl]-1-ethoxy]phenyl]butanoate, the title compound (30 mg) was obtained as a yellow foam: MS (ES) m/e 327 (M+H)⁺. Anal. Calcd for C₁₉H₂₂N₂O₃.0.60 HCl.0.55 H₂O: C, 63.71; H, 6.67; N, 7.82. Found: C, 63.41; H, 6.78; N, 8.14.

### EXAMPLE 44

Parenteral Dosage Unit Composition

A preparation which contains 20 mg of the compound of Example 1 as a sterile dry powder is prepared as follows: 20 mg of the compound is dissolved in 15 mL of distilled water. The solution is filtered under sterile conditions into a 25 mL multi-dose ampoule and lyophilized. The powder is reconstituted by addition of 20 mL of 5% dextrose in water (D5W) for intravenous or intramuscular injection. The dosage is thereby determined by the injection volume. Subsequent dilution may be made by addition of a metered volume of this dosage unit to another volume of D5W for injection, or a metered dose may be added to another mechanism for dispensing the drug, as in a bottle or bag for IV drip infusion or other injection-infusion system.

### EXAMPLE 45

Oral Dosage Unit Composition

A capsule for oral administration is prepared by mixing and milling 50 mg of the compound of Example 1 with 75 mg of lactose and 5 mg of magnesium stearate. The resulting powder is screened and filled into a hard gelatin capsule.

### EXAMPLE 46

Oral Dosage Unit Composition

A tablet for oral administration is prepared by mixing and granulating 20 mg of sucrose, 150 mg of calcium sulfate dihydrate and 50 mg of the compound of Example 1 with a 10% gelatin solution. The wet granules are screened, dried, mixed with 10 mg starch, 5 mg talc and 3 mg stearic acid; and compressed into a tablet.

The above description fully discloses how to make and use the present invention. However, the present invention is not limited to the particular embodiments described hereinabove, but includes all modifications thereof within the scope of the following claims. The various references to journals, patents and other publications which are cited herein comprises the state of the art and are incorporated herein by reference as though fully set forth.

What is claimed is:

1. A compound according to formula (I): 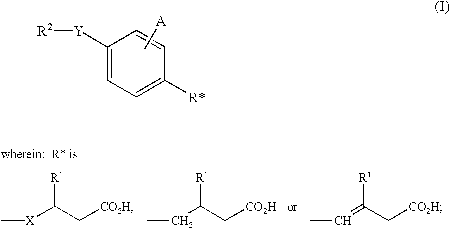
X is CR′R′, NR′, O or S;
Y is CR′R′, NR′, O or S;
A is H, halo, —OR^{g}, —SR^{g}, —CN, —NR^{g}R^{k}, —NO₂, —CF₃, —S(O)ᵣCF₃, —CO₂R^{g}, —COR^{g}, —CONR^{g}₂—C₁₋₆alkyl, —C₀₋₆alkyl-Ar, —C₀₋₆alkyl-Het, —C₀₋₆alkyl-C₃₋₆cycloalkyl, —S(O)ₖR^{g}, or CH₂N(R^{f})₂;
R¹ is —C₀₋₆alkyl-Het-, —C₀₋₆alkyl-Ar, —C₁₋₆alkyl, —H, —CN, —CH═CH₂, —C≡CH or, —S(O)ₖR^{g};
R²is 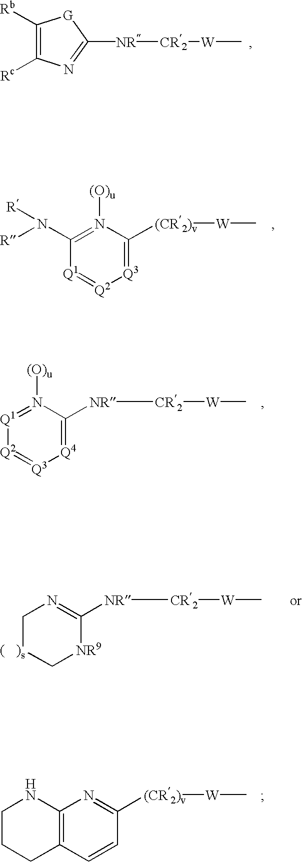
W is —(CHR^{g})ₐ—U—(CHR^{g})_{b}—;
U is absent or CO, CR^{g}₂, C(═CR^{g}₂), S(O)ₖ, O, NR^{g}, CR^{g}OR^{g}, CR^{g}(OR^{k})CR^{g}₂, CR^{g}₂CR^{g}(OR^{k}), C(O)CR^{g}₂, CR^{g}₂C(O), CONRⁱ, NRⁱCO, OC(O), C(O)O, C(S)O, OC(S), C(S)NR^{g}, NR^{g}C(S), S(O)₂NR^{g}, NR^{g}S(O)₂N═N, NR^{g}NR^{g}, NR^{g}CR^{g}₂, CR^{g}₂NR^{g}, CR^{g}₂O, OCR^{g}₂, C≡C , CR^{g}═CR^{g}, Ar or Het;
G is NR^{e}, S or O;
R^{g} is H, C₁₋₆alkyl, Het-C₀₋₆alkyl, C₃₋₇cycloalkyl-C₀₋₆alkyl or Ar—C₀₋₆alkyl;
R^{k} is R^{g}, —C(O)R^{g}, or —C(O)OR^{f};
Rⁱ is is H, C₁₋₆alkyl, Het-C₀₋₆alkyl, C₃₋₇cycloalkyl-C₀₋₆alkyl, Ar—C₀₋₆alkyl, or C₁₋₆alkyl substituted by one to three groups chosen from halogen, CN, NR^{g}₂, OR^{g}, SR^{g}, CO₂R^{g}, and CON(R^{g})₂;
R^{f} is H, C₁₋₆alkyl or Ar—C₀₋₆alkyl;
R^{e} is H, C₁₋₆alkyl, Ar—C₀₋₆alkyl, Het-C₀₋₆alkyl, C₃₋₇cycloalkyl-C₀₋₆alkyl, or (CH₂)ₖCO₂R_{g};
R^{b} and R^{c} are independently selected from H, C₁₋₆alkyl, Ar—C₀₋₆alkyl, Het-C₀₋₆alkyl, or C₃₋₆cycloalkyl-C₀₋₆alkyl, halogen, CF₃, OR^{f}, S(O)ₖR^{f}, COR^{f}, NO₂, N(R^{f})₂, CO(NR^{f})₂, CH₂N(R^{f})₂, or R^{b} and R^{c} are joined together to form a five or six membered aromatic or non-aromatic carbocyclic or heterocyclic ring, optionally substituted by up to three substituents chosen from halogen, CF₃, C₁₋₄alkyl, OR^{f}, S(O)ₖR^{f}, COR^{f}, CO₂R^{f}, OH, NO₂, N(R^{f})₂, CO(NR^{f})₂, and CH₂N(R^{f})₂; or methylenedioxy;
Q¹, Q², Q³ and Q⁴ are independently N or C—R^{y}, provided that no more than one of Q¹, Q², Q³ and Q⁴ is N;
R′ is H, C₁₋₆alkyl, Ar—C₀₋₆alkyl or C₃₋₆cycloalkyl-C0-6alkyl;
R″ is R′, —C(O)R′ or —C(O)OR′;
R^{y} is H, halo, —OR^{g}, —SR^{g}, —CN, —NR^{g}R^{k}, —NO₂, —CF₃, CF₃S(O)ᵣ—, —CO₂R^{g}, —COR^{g} or —CONR^{g}₂, or C₁₋₆alkyl optionally substituted by halo, —OR^{g}, —SR^{g}, —CN, —NR^{g}R″, —NO₂, —CF₃, R′S(O)ᵣ—, —CO₂R^{g}, —COR^{g} or —CONR^{g}₂;
a is 0, 1 or 2;
b is 0, 1 or 2;
k is 0, 1 or 2;
r is 0, 1 or 2;
s is 0, 1 or 2;
u is 0 or 1; and
v is 0 or 1;
or a pharmaceutically acceptable salt thereof.

2. A compound according to formula (Ia): 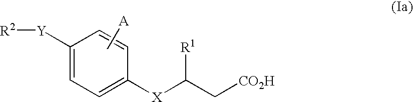
wherein:
X is CR′R′, NR′, O or S;
Y is CR′R′, NR′, O or S;
A is H, halo, —OR^{g}, —SR^{g}, —CN, —NR^{g}R^{k}, —NO₂, —CF₃, —S(O)ᵣCF₃, —CO₂R^{g}, —COR^{g}, —CONR^{g}₂—C₁₋₆akyl, —C₀₋₆alkyl-Ar, —C₀₋₆alkyl-Het, —C₀₋₆alkyl-C₃₋₆cycloalkyl, —S(O)ₖR^{g}, or CH₂N(R^{f})₂;
R¹ is —C₀₋₆alkyl-Het-, —C₀₋₆alkyl-Ar, H, —CN or —S(O)ₖR^{g}; 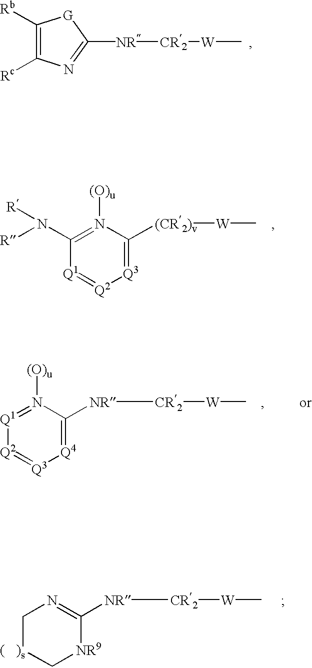
W is —(CHR^{g})ₐ—U—(CHR^{g})_{b}—;
U is absent or CO, CR^{g}₂, C(═CR^{g}₂), S(O)ₖ, O, NR^{g}, CR^{g}OR^{g}, CR^{g}(OR^{k})CR^{g}₂, CR^{g}₂CR^{g}(OR^{k}), C(O)CR^{g}₂, CR^{g}₂C(O), CONRⁱ, NRⁱCO, OC(O), C(O)O, C(S)O, OC(S), C(S)NR^{g}, NR^{g}C(S), S(O)₂NR^{g}, NR^{g}S(O)₂ N═N, NR^{g}NR^{g}, NR^{g}CR^{g}₂, CR^{g}₂NR^{g}, CR^{g}₂O, OCR^{g}₂, C≡C , CR^{g}═CR^{g}, Ar or Het;
G is NR^{e}, S or O;
R^{g} is H, C₁₋₆alkyl, Het-C₀₋₆alkyl, C₃₋₇cycloalkyl-C₀₋₆alkyl or Ar—C₀₋₆alkyl;
R^{k} is R^{g}, —C(O)R^{g}, or —C(O)OR^{f};
Rⁱ is is H, C₁₋₆alkyl, Het-C₀₋₆alkyl, C₃₋₇cycloalkyl-C₀₋₆alkyl, Ar—C₀₋₆alkyl, or C₁₋₆alkyl substituted by one to three groups chosen from halogen, CN, NR^{g}₂, OR^{g}, SR^{g}, CO₂R^{g}, and CON(R^{g})₂;
R^{f} is H, C₁₋₆alkyl or Ar—C₀₋₆alkyl;
R^{e} is H, C₁₋₆alkyl, Ar—C₀₋₆alkyl, Het-C₀₋₆alkyl, C₃₋₇cycloalkyl-C₀₋₆alkyl, or (CH₂)ₖCO₂R^{g};
R^{b} and R^{c} are independently selected from H, C₁₋₆alkyl, Ar—C₀₋₆alkyl, Het-C₀₋₆alkyl, or C₃₋₆cycloalkyl-C₀₋₆alkyl, halogen, CF₃, OR^{f}, S(O)ₖR^{f}, COR^{f}, NO₂, N(R^{f})₂, CO(NR^{f})₂, CH₂N(R^{f})₂, or R^{b} and R^{c} are joined together to form a five or six membered aromatic or non-aromatic carbocyclic or heterocyclic ring, optionally substituted by up to three substituents chosen from halogen, CF₃, C₁₋₄alkyl, OR^{f}, S(O)ₖR^{f}, COR^{f}, CO₂R^{f}, OH, NO₂, N(R^{f})₂, CO(NR^{f})₂, and CH₂N(R^{f})₂; or methylenedioxy;
Q¹, Q², Q³ and Q⁴ are independently N or C—R^{y}, provided that no more than one of Q¹, Q², Q³ and Q⁴ is N;
R′ is H, C₁₋₆alkyl, Ar—C₀₋₆alkyl or C₃₋₆cycloalkyl-C₀₋₆alkyl;
R″ is R′, —C(O)R′ or —C(O)OR′,
R^{y} is H, halo, —OR^{g}, —SR^{g}, —CN, —NR^{g}R^{k}, —NO₂, —CF₃—, CF₃S(O)ᵣ—, —CO₂R^{g}, —COR^{g} or —CONR^{g}₂, or C₁₋₆alkyl optionally substituted by halo, —OR^{g}, —SR^{g}, —CN, —NR^{g}R″, —NO₂, —CF₃, R′S(O)ᵣ—, —CO₂R^{g}, —COR^{g} or —CONR^{g}₂;
a is 0, 1 or 2;
b is 0, 1 or 2;
k is 0, 1 or 2;
r is 0, 1 or 2;
s is 0, 1 or 2;
u is 0 or 1; and
v is 0 or 1;
or a pharmaceutically acceptable salt thereof.

3. A compound according to claim 1 or 2 in which R² is 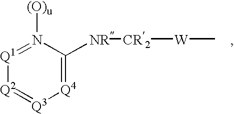
wherein Q¹, Q², and Q³ are each CR^{y}, Q⁴ is CR^{y} or N and u is 0.

4. A compound according to claim 3 in which each R′ is H, R″ is H or C₁₋₆alkyl, W is —(CH₂)₁₋₄—, Q⁴ is CR^{y} and R^{y} is H.

5. A compound according to claim 1 or 2 in which R² is 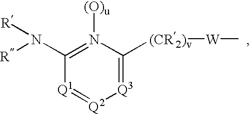
wherein Q¹, Q², and Q³ are each CH and u is 0.

6. A compound according to claim 5 in which each R′ is H, R″ is H or C₁₋₆alkyl, v is 0 and W is —CH₂—CH₂—.

7. A compound according to claim 1 or 2 in which R² is 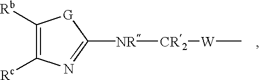
wherein G is NH and R^{b} and R^{c} are each H.

8. A compound according to claim 7 in which W is —CH₂—CH₂—.

9. A compound according to claim 1 or 2 in which R² is 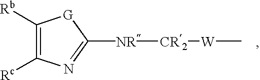
wherein G is NH and R^{b} and R^{c} are joined together to form a five or six membered aromatic or non-aromatic carbocyclic or heterocyclic ring, optionally substituted by up to three substituents chosen from halogen, CF₃, C₁₋₄alkyl, OR^{f}, S(O)ₖR^{f}, COR^{f}, CO₂R^{f}, OH, NO₂, N(R^{f})₂, CO(NR^{f})₂, and CH₂N(R^{f})₂; or methylenedioxy.

10. A compound according to claim 9 in which R^{b} and R^{c} are joined together to form a six membered aromatic carbocyclic ring.

11. A compound according to claim 10 in which W is —CH₂—CH₂—.

12. A compound according to claim 9 in which R^{b} and R^{c} are joined together to form a six membered aromatic heterocyclic ring.

13. A compound according to claim 12 in which W is —CH₂—CH₂—.

14. A compound according to claim 1 or 2 in which R² is 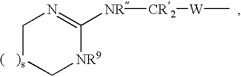
wherein each R′ is H, R″ is H or C₁₋₆alkyl, R^{g} is H or C₁₋₆alkyl and s is 0, 1 or 2.

15. A compound according to claim 14 in which W is —CH₂—CH₂—.

16. A compound according to claim 1 or 2 in which R¹ is phenyl, benzyl, pyridyl, imidazolyl, oxazolyl or thiazolyl.

17. A compound according to claim 1 or 2 in which Y is O or CH₂.

18. A compound according to claim 1 or 2 in which X is NH or CH₂.

19. A compound according to claim 1 in which R² is 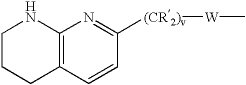
wherein v is 0 and W is —CH₂—CH₂.

20. A compound which is:
(±)-3-phenyl-4-[4-[3-(pyridin-2-yl)amino-1-propyloxy]phenyl]butanoic acid;
(±)-3-phenyl-4-[4-[2-[6-(methylamino)pyridin-2-yl]-1-ethoxy]phenyl]butanoic acid;
(±)-3-phenyl-3-[4-[4-(pyridin-2-yl)amino-1-butyl]phenylamino]propanoic acid;
4-[4-[2-[6-(methylamino)pyridin-2-yl]-1-ethoxy]phenyl]butanoic acid;
(S)-3-phenyl-4-[4-[3-(pyridin-2-yl)amino-1-propyloxy]phenyl]butanoic acid;
2-phenoxy-4-[5-(pyridin-2-yl)amino-1-pentyloxy]phenylacetic acid;
4-[4-[6-(methylamino)pyridin-2-yl]-1-ethoxy]-2-phenoxyphenyl]butanoic acid;
(±)-4-[4-[6-(methylamino)pyrdin-2-yl]-1-ethoxy]phenyl]-3-vinylbutanoic acid;
(±)-3-methyl-4-[4-[3-(pyridin-2-yl)amino-1-propyloxy]phenyl]butanoic acid;
(R)-3-phenyl-4-[4-[3-(pyridin-2-yl)amino-1-propyloxy]phenyl]butanoic acid;
(±)-4-[4-[2-[6-(methylamino)pyridin-2-yl]-1-ethoxy]phenyl]-3-(pyridin-2-yl)butanoic acid;
(±)-3-methyl-4-[4-[2-[2-(methylamino)pyridin-5-yl]-1-ethoxy]phenyl]butanoic acid;
2-[N-benzyl-N-[4-[2-[6-(methylamino)pyridin-2-yl]-1-ethoxy]benzyl]amino]acetic acid;
(±)-4-[4-[2-[6-(methylamino)pyridin-2-yl]-1-ethoxy]phenyl]-3-(thiophen-2-yl)butanoic acid;
2-[N-[4-[2-[6-(methylamino)pyridin-2-yl]-1-ethoxy]benzyl]-N-phenyl]amino]-acetic acid;
(±)-3-(4-bromophenyl)-4-[4-[2-[6-(methylamino)pyridin-2-yl]-1-ethoxy]phenyl]-butanoic acid;
(±)-3-methyl-4-[4-[2-[6-(methylamino)pyridin-2-yl]-1-ethoxy]phenyl]butanoic acid;
(S)-3-phenyl-4-[4-[2-[6-(methylamino)pyridin-2-yl]-1-ethoxy]phenyl]butanoic acid;
(±)-3-(4-isopropylphenyl)-4-[4-[2-[6-(methylamino)pyridin-2-yl]-1-ethoxy]-phenyl]butanoic acid;
(±)-3-(4-isopropylphenyl)-4-[4-[3-(4-methylpyridin-2-yl)amino-1-propyloxy]phenyl]butanoic acid;
(±)-4-[4-[2-[6-(methylamino)pyridin-2-yl]-1-ethoxy]phenyl]-3-(oxazol-2-yl)butanoic acid;
2-[N-[2-methoxy-4-[2-[6-(methylamino)pyridin-2-yl]-1-ethoxy]-benzyl]amino]acetic acid;
4-[4-[2-[6-(methylamino)pyridin-2-yl]-1-ethoxy]phenyl]but-3-enoic acid;
(±)-4-[4-[2-[6-(methylamino)pyridin-2-yl]-1-ethoxy]phenyl]-3-(thiazol-2-yl)butanoic acid;
(±)-3-phenyl-4-[4-[[2-(pyridin-2-yl)amino-1-ethylamino]carbonyl]phenyl]butanoic acid;
(±)-3-(furan-2-yl)-4-[4-[2-[6-(methylamino)pyridin-2-yl]-1-ethoxy]phenyl]-butanoic acid;
(±)-4-[4-[2-[6-(methylamino)pyridin-2-yl]-1-ethoxy]phenyl]-3-(2-phenylethyl)-butanoic acid;
(S)-3-phenyl-4-[4-[2-(5,6,7,8-tetrahydro-1,8-naphthyridin-2-yl)-1-ethoxy]-phenyl]butanoic acid;
3-methyl-4-[4-[2-[6-(methylamino)pyridin-2-yl]-1-ethoxy]phenyl]-3-butenoic acid;
(±)-3-[1-(dimethylaminosulfonyl)imidazol-2-yl]-4-[4-[2-[6-(methylamino)pyridin-2-yl]-1-ethoxy]phenyl]butanoic acid;
(±)-3-benzyl-4-[4-[2-[6-(methylamino)pyridin-2-yl]-1-ethoxy]phenyl]butanoic acid;
(±)-3-(imidazol-2-yl)-4-[4-[2-[6-(methylamino)pyridin-2-yl]-1-ethoxy]phenyl]-butanoic acid
(S)-4-[4-[2-[6-(methylamino)pyridin-2-yl]-1-ethoxy]phenyl]-3-(thiazol-2-yl)butanoic acid;
(R)-4-[4-[2-[6-(methylamino)pyridin-2-yl]-1-ethoxy]phenyl]-3-(thiazol-2-yl)butanoic acid;
(S)-3-phenyl-4-[4-[3-(3,4,5,6-tetrahydropyrimidin-2-yl)amino-1-propyloxy]-phenyl]butanoic acid;
(±)-3-cyclopropyl-4-[4-[2-[6-(methylamino)pyridin-2-yl]-1-ethoxy]phenyl]-butanoic acid;
(±)-3-(benzothiazol-2-yl)-4-[4-[2-[6-(methylamino)pyridin-2-yl]-1-ethoxy]phenyl]-butanoic acid;
(S)-4-[4-[2-(5,6,7,8-tetrahydro-1,8-naphthyridin-2-yl)-1-ethoxy]phenyl]-3-(thiazol-2-yl)-butanoic acid;
(±)-3-(4-methylthiazol-2-yl)-4-[4-[2-[6-(methylamino)pyridin-2-yl]-1-ethoxy]-phenyl]butanoic acid;
(±)-3-[4-carboxy-1,3-oxazol-2-yl]-4-[4-[2-[6-(methylamino)pyridin-2-yl]-1-ethoxy]phenyl]butanoic acid;
(±)-3-[4-(aminocarbonyl)-1,3-oxazol-2-yl]-4-[4-[2-[6-(methylamino)pyridin-2-yl]-1-ethoxy]phenyl]butanoic acid;
(±)-3-[4-(dimethylaminocarbonyl)-1,3-oxazol-2-yl]-4-[4-[2-[6-(methylamino)-pyridin-2-yl]-1-ethoxy]phenyl]butanoic acid;
(±)-3-[4-[2-[6-(methylamino)pyridin-2-yl]ethoxy]benzyl]pent-4-ynoic acid;
or a pharmaceutically acceptable salt thereof.

21. A pharmaceutical composition which comprises a compound according to claims 1-20 and a pharmaceutically acceptable carrier.

22. A pharmaceutical composition which comprises a compound according to claims 1-20, an antineoplastic agent and a pharmaceutically acceptable carrier.

23. The pharmaceutical composition according to claim 22 wherein the antineoplastic agent is topotecan or cisplatin.

24. A pharmaceutical composition which comprises a compound according to claim 1, an inhibitor of bone resorption and a pharmaceutically acceptable carrier.

25. A method of treating a disease state in which antagonism of the α_{V}β₃ receptor is indicated which comprises administering to a subject in need thereof a compound according to claim 1.

26. A method of treating a disease state in which antagonism of the α_{V}β₅ receptor is indicated which comprises administering to a subject in need thereof a compound according to claim 1.

27. A method of treating osteoporosis which comprises administering to a subject in need thereof a compound according to claim 1.

28. A method for inhibiting angiogenesis, tumor growth or tumor metastasis which comprises administering to a subject in need thereof a compound according to claim 1.

29. A method of treating atherosclerosis, restenosis or inflammation which comprises administering to a subject in need thereof a compound according to claim 1.

30. A method of inhibiting tumor growth which comprises administering stepwise or in physical combination a compound according to claim 1 and an antineoplastic agent.

31. The method according to claim 30 wherein the antineoplastic agent is topotecan or cisplatin.

32. A method of treating osteoporosis or inhibiting bone loss which comprises administering stepwise or in physical combination a compound according to claim 1 and an inhibitor of bone resorption.

33. A compound according to formula (II): 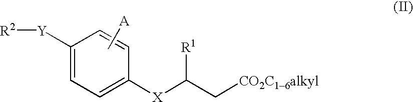
wherein:
X is CR′R′, NR′, O or S;
Y is CR′R′, NR′, O or S;
A is H, halo, —OR^{g}, —SR^{g}, —CN, —NR^{g}R^{k}, —NO₂, —CF₃, —S(O)ᵣCF₃, —CO₂R^{g}, —COR^{g}, —CONR^{g}₂—C₁₋₆alkyl, —C₀₋₆alkyl-Ar, —C₀₋₆alkyl-Het, —C₀₋₆alkyl-C₃₋₆cycloalkyl, —S(O)ₖR^{g}, or CH₂N(R^{f})₂;
R¹ is —C₀₋₆alkyl-Het-, —C₀₋₆alkyl-Ar, H, —CN or —S(O)ₖR^{g};
R² is 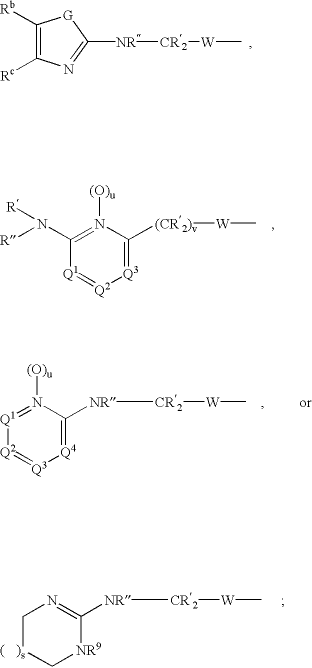
W is —(CHR^{g})ₐ—U—(CHR^{g})_{b}—;
U is absent or CO, CR^{g}₂, C(═CR^{g}₂), S(O)ₖ, O, NR^{g}, CR^{g}OR^{g}, CR^{g}(OR^{k})CR^{g}₂, CR^{g}₂CR^{g}(OR^{k}), C(O)CR^{g}₂, CR^{g}₂C(O), CONRⁱ, NRⁱCO, OC(O), C(O)O, C(S)O, OC(S), C(S)NR^{g}, NR^{g}C(S), S(O)₂NR^{g}, NR^{g}S(O)₂ N═N, NR^{g}NR^{g}, NR^{g}CR^{g}₂, CR^{g}₂NR^{g}, CR^{g}₂O, OCR^{g}₂, C≡C , CR^{g}═CR^{g}, Ar or Het;
G is NR^{e}, S or O;
R^{g} is H, C₁₋₆alkyl, Het-C₀₋₆alkyl, C₃₋₇cycloalkyl-C₀₋₆alkyl or Ar—C₀₋₆alkyl;
R^{k} is R^{g}, —C(O)R^{g}, or —C(O)OR^{f};
Rⁱ is is H, C₁₋₆alkyl, Het-C₀₋₆alkyl, C₃₋₇cycloalkyl-C₀₋₆alkyl, Ar—C₀₋₆alkyl, or C₁₋₆alkyl substituted by one to three groups chosen from halogen, CN, NR^{g}₂, OR^{g}, SR^{g}, CO₂R^{g}, and CON(R^{g})₂;
R^{f} is H, C₁₋₆alkyl or Ar—C₀₋₆alkyl;
R^{e} is H, C₁₋₆alkyl, Ar—C₀₋₆alkyl, Het-C₀₋₆alkyl, C₃₋₇cycloalkyl-C₀₋₆alkyl, or (CH₂)ₖCO₂R_{g};
R^{b} and R^{c} are independently selected from H, C₁₋₆alkyl, Ar—C₀₋₆alkyl, Het-C₀₋₆alkyl, or C₃₋₆cycloalkyl-C₀₋₆alkyl, halogen, CF₃, OR^{f}, S(O)ₖR^{f}, COR^{f}, NO₂, N(R^{f})₂, CO(NR^{f})₂, CH₂N(R^{f})₂, or R^{b} and R^{c} are joined together to form a five or six membered aromatic or non-aromatic carbocyclic or heterocyclic ring, optionally substituted by up to three substituents chosen from halogen, CF₃, C₁₋₄alkyl, OR^{f}, S(O)ₖR^{f}, COR^{f}, CO₂R^{f}, OH, NO₂, N(R^{f})₂, CO(NR^{f})₂, and CH₂N(R^{f})₂; or methylenedioxy;
Q¹, Q², Q³ and Q⁴ are independently N or C—R^{y}, provided that no more than one of Q¹, Q², Q³ and Q⁴ is N;
R′ is H, C₁₋₆alkyl, Ar—C₀₋₆alkyl or C₃₋₆cycloalkyl-C₀₋₆alkyl;
R″ is R′, —C(O)R′ or —C(O)OR′;
R^{y} is H, halo, —OR^{g}, —SR^{g}, —CN, —NR^{g}R^{k}, —NO₂, —CF₃, CF₃S(O)ᵣ—, —CO₂R^{g}, —COR^{g} or —CONR^{g}₂, or C₁₋₆alkyl optionally substituted by halo, —OR^{g}, —SR^{g}, —CN, —NR^{g}R″, —NO₂, —CF₃, R′S(O)ᵣ—, —CO₂R^{g}, —COR^{g} or —CONR^{g}₂;
a is 0, 1 or 2;
b is 0, 1 or 2;
k is 0, 1 or 2;
r is 0, 1 or 2;
s is 0, 1 or 2;
u is 0 or 1; and
v is 0 or 1;
or a pharmaceutically acceptable salt thereof; or
a compound according to formula (III): 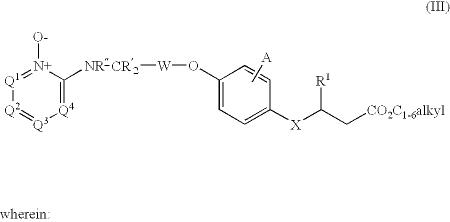
X is CR′R′, NR′, O or S;
Y is CR′R′, NR′, O or S;
A is H, halo, —OR^{g}, —SR^{g}, —CN, —NR^{g}R^{k}, —NO₂, —CF₃, —S(O)ᵣCF₃, —CO₂R^{g}, —COR^{g}, —CONR^{g}₂—C₁₋₆alkyl, —C₀₋₆alkyl-Ar, —C₀₋₆alkyl-Het, —C₀₋₆alkyl-C₃₋₆cycloalkyl, —S(O)ₖR^{g}, or CH₂N(R^{f})₂;
R¹ is —C₀₋₆alkyl-Het-, —C₀₋₆alkyl-Ar, H, —CN or —S(O)ₖR^{g};
W is —(CHR^{g})ₐ—U—(CHR^{g})_{b}—;
U is absent or CO, CR^{g}₂, C(═CR^{g}₂), S(O)ₖ, O, NR^{g}, CR^{g}OR^{g}, CR^{g}(OR^{k})CR^{g}₂, CR^{g}₂CR^{g}(OR^{k}), C(O)CR^{g}₂, CR^{g}₂C(O), CONRⁱ, NRⁱCO, OC(O), C(O)O, C(S)O, OC(S), C(S)NR^{g}, NR^{g}C(S), S(O)₂NR^{g}, NR^{g}S(O)₂ N═N, NR^{g}NR^{g}, NR^{g}CR^{g}₂, CR^{g}₂NR^{g}, CR^{g}₂O, OCR^{g}₂, C≡C , CR^{g}═CR^{g}, Ar or Het;
R^{g} is H, C₁₋₆alkyl, Het-C₀₋₆alkyl, C₃₋₇cycloalkyl-C₀₋₆alkyl or Ar—C₀₋₆alkyl;
R^{k} is R^{g}, —C(O)R^{g}, or —C(O)OR^{f};
Rⁱ is is H, C₁₋₆alkyl, Het-C₀₋₆alkyl, C₃₋₇cycloalkyl-C₀₋₆alkyl, Ar—C₀₋₆alkyl, or C₁₋₆alkyl substituted by one to three groups chosen from halogen, CN, NR^{g}₂, OR^{g}, SR^{g}, CO₂R^{g}, and CON(R^{g})₂;
R^{f} is H, C₁₋₆alkyl or Ar—C₀₋₆alkyl;
Q¹, Q², Q³ and Q⁴ are independently N or C—R^{y}, provided that no more than one of Q¹, Q², Q³ and Q⁴ is N;
R′ is H, C₁₋₆alkyl, Ar—C₀₋₆alkyl or C₃₋₆cycloalkyl-C₀₋₆alkyl;
R″ is R′, —C(O)R′ or —C(O)OR′;
R^{y} is H, halo, —OR^{g}, —SR^{g}, —CN, —NR^{g}R^{k}, —NO₂, —CF₃, CF₃S(O)ᵣ—, —CO₂R^{g}, —COR^{g} or —CONR^{g}₂, or C₁₋₆alkyl optionally substituted by halo, —OR^{g}, —SR^{g}, —CN, —NR^{g}R″, —NO₂, —CF₃, R′S(O)ᵣ—, —CO₂R^{g}, —COR^{g} or —CONR^{g}₂;
a is 0, 1 or 2; and
b is 0, 1 or 2;
or a pharmaceutically acceptable salt thereof.

34. A process for preparing a compound of the formula (Ia) as defined in claim 2, which process comprises reacting a compound of formula (IV) with a compound of formula (V): 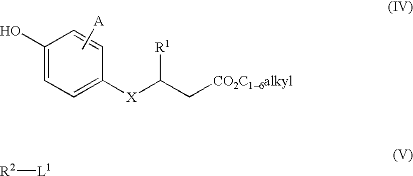
wherein R¹, A and X are as defined in formula (Ia), with any reactive functional groups protected, and L¹ is OH or halo;
and thereafter removing any protecting groups, and optionally forming a pharmaceutically acceptable salt.

35. A process for preparing a compound of the formula (Ia) as defined in claim 2, which process comprises reacting a compound of formula (IV) with a compound of formula (VI): 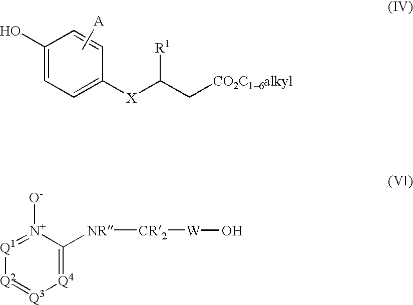
wherein R¹, A, X, R′, R″, W, Q¹, Q², Q³ and Q⁴ are as defined in formula (Ia), with any reactive functional groups protected;
and thereafter removing any protecting groups, and optionally forming a pharmaceutically acceptable salt; or
a process for preparing a compound of the formula (Ia) as defined in claim 2, which process comprises reacting a compound of formula (IV) with a compound of formula (VII): 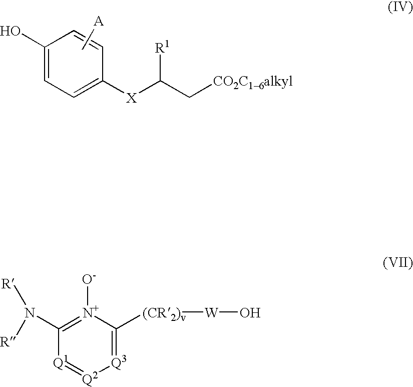
wherein R¹, A, X, R′, R″, W, Q¹, Q², Q³ and v are as defined in formula (Ia), with any reactive functional groups protected;
and thereafter removing any protecting groups, and optionally forming a pharmaceutically acceptable salt.

36. A compound according to any one of claims 1 to 20 for use as a medicament.

37. The use of a compound of the formula (I) as defined in claim 1 in the manufacture of a medicament for the treatment of diseases in which antagonism of the α_{V}β₃ receptor is indicated.

38. The use of a compound of the formula (I) as defined in claim 1 in the manufacture of a medicament for the treatment of diseases in which antagonism of the α_{V}β₅ receptor is indicated.

39. The use of a compound of the formula (I) as defined in claim 1 in the manufacture of a medicament for the treatment of osteoporosis.

40. The use of a compound of the formula (I) as defined in claim 1 in the manufacture of a medicament for the inhibition of angiogenesis, tumor growth or tumor metastasis.

41. The use of a compound of the formula (I) as defined in claim 1 in the manufacture of a medicament for the treatment of atherosclerosis, restenosis or inflammation.

42. The use of a compound of the formula (I) as defined in claim 1 and an antineoplastic agent in the manufacture of a medicament for the inhibition of tumor growth in physical combination or for stepwise administration.

43. The use according to claim 43 wherein the antineoplastic agent is topotecan or cisplatin.
